(19) United States
(12) Patent Application Publication
(10) Pub. No.: US 2026/0224664 A1
(43) Pub. Date: Aug. 6, 2026

(54) ENGINEERED NUCLEIC ACIDS AND METHODS OF USE THEREOF

(71) Applicant: Moderna TX, Inc., Cambridge, MA (US)

(72) Inventors: Jason P. Schrum, Philadelphia, PA (US); Stephane Bancel, Cambridge, MA (US); Noubar B. Afeyan, Cambridge, MA (US); Kenechi Ejebe, New York, NY (US)

(21) Appl. No.: 19/314,261

(22) Filed: Aug. 29, 2025

Related U.S. Application Data
(63) Continuation of application No. 19/016,971, filed on Jan. 10, 2025, now abandoned.
Application No. 19/016,971 is a continuation of application No. 17/528,588, filed on Nov. 17, 2021, now abandoned.
Application No. 17/528,588 is a continuation of application No. 15/914,615, filed on Mar. 7, 2018, now abandoned.
Application No. 15/914,615 is a continuation of application No. 15/248,053, filed on Aug. 26, 2016, now Pat. No. 9,937,233.
Application No. 15/248,053 is a continuation of application No. 14/878,286, filed on Oct. 8, 2015, now Pat. No. 9,447,164.
Application No. 14/878,286 is a continuation of application No. 14/270,736, filed on May 6, 2014, now Pat. No. 9,181,319.
Application No. 14/270,736 is a division of application No. 13/204,609, filed on Aug. 5, 2011, now Pat. No. 8,822,663.
(60) Provisional application No. 61/401,052, filed on Aug. 6, 2010.

(51) Int. Cl. A61K 38/19; A61K 31/395; A61K 31/495; A61K 31/7088; A61K 31/7115; A61K 38/00; A61K 38/13; A61K 38/28; A61K 39/395; A61K 48/00; C07K 14/62; C07K 14/685

(52) U.S. Cl. CPC A61K 38/193; A61K 31/395; A61K 31/495; A61K 31/7088; A61K 31/7115; A61K 38/13; A61K 38/195; A61K 38/28; A61K 39/3955; A61K 48/0066; A61K 48/0075; A61K 48/0083; C07K 14/62; C07K 14/685; A61K 38/00; A61K 48/00

(57) ABSTRACT

Provided are compositions and methods for delivering biological moieties such as modified nucleic acids into cells to modulate protein expression. Such compositions and methods include the use of modified messenger RNAs, and are useful to treat or prevent diseases, disorders or conditions, or to improve a subject's heath or wellbeing.

### REFERENCE TO SEQUENCE LISTING

The present application is being filed along with a Sequence Listing in XML format. The Sequence Listing file, entitled 50858-141009_Sequence_Listing_8_26_25.xml, was created on Aug. 26, 2025, and is 529,215 bytes in size. The information in XML format of the Sequence Listing is incorporated herein by reference in its entirety.

### BACKGROUND OF THE INVENTION

Naturally occurring RNAs are synthesized from four basic ribonucleotides: ATP, CTP, UTP and GTP, but may contain post-transcriptionally modified nucleotides. Further, approximately one hundred different nucleoside modifications have been identified in RNA (Rozenski, J, Crain, P, and McCloskey, J. (1999). The RNA Modification Database: 1999 update. Nucl Acids Res 27:196-197). The role of nucleoside modifications on the immuno-stimulatory potential and on the translation efficiency of RNA, however, is unclear.

There is a need in the art for biological modalities to address the modulation of intracellular translation of nucleic acids.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described herein. The materials, methods, and examples are illustrative only and not intended to be limiting. Other features of the disclosure are apparent from the following detailed description and the claims.

### SUMMARY OF THE INVENTION

Provided herein are modified nucleic acids encoding melanocyte-stimulating hormone (MSH), insulin, and granulocyte colony-stimulating factor (G-CSF), precursors thereof or partially or fully processed forms of these proteins or these precursors. In certain embodiments, the modified nucleic acids comprise mRNA. In particular embodiments, the modified mRNA (mmRNA) is derived from cDNA. In certain embodiments, the mmRNA comprises at least two nucleoside modifications. In certain embodiments, these nucleoside modifications are 5-methylcytosin and pseudouridine.

Further, provided herein are formulations comprising the modified nucleic acids described herein. In certain embodiments, the formulations further comprise a pharmaceutically acceptable carrier. In certain embodiments, the carrier is formulated for systemic or local administration. In certain embodiments, the administration is oral or topical. In certain embodiments, the compositions comprise naked modified nucleic acids. In other embodiments, the modified nucleic acids are complexed or encapsulated.

Provided herein are methods of treating a subject having or being suspected of having a disease, the methods comprising administering to a subject in need of such treatment a formulation described herein in an amount sufficient to treat the disease. In specific embodiments, the disease is associated with one or more cellular and/or molecular changes affecting, for example, the level, activity, and/or localization of precursors of melanocyte-stimulating hormone (MSH), insulin, and granulocyte colony-stimulating factor (G-CSF), or a partially or fully processed form of these precursors. In certain embodiments, the methods of treating a subject having or being suspected of having a disease comprise administering to the subject in need of such treatment a formulation comprising a modified nucleic acid described herein in an amount sufficient to modulate one or more activities associated with MSH, G-CSF or insulin to treat the disease.

Provided herein are pharmaceutical formulations comprising: (i) an effective amount of a synthetic messenger ribonucleic acid (mRNA) encoding a granulocyte colony-stimulating factor (G-CSF) polypeptide; and (ii) a pharmaceutically acceptable carrier, wherein i) the mRNA comprises pseudouridine, 5′methyl-cytidine or a combination thereof, or ii) wherein the mRNA does not comprise a substantial amount of a nucleotide or nucleotides selected from the group consisting of uridine, cytidine, and a combination of uridine and cytidine, and wherein the formulation is suitable for repeated intravenous administration to a mammalian subject in need thereof. In certain embodiments, the G-CSF polypeptide comprises: (a) the amino acid sequence of SEQ ID NOs: 13 or 14; (b) an amino acid sequence at least 95% identical to SEQ ID NOs: 13 or 14; (c) a fragment of SEQ ID NOs: 13 or 14; (d) the amino acid sequence encoded by the nucleic acid of SEQ ID NO: 2 or SEQ ID NO: 17; or (e) the amino acid sequence encoded by a nucleic acid at least 95% identical to SEQ ID NO: 2 or SEQ ID NO: 17. In some embodiments, the pharmaceutical formulation provided herein further comprise a lipid-based transfection reagent. In some embodiments, the synthetic messenger ribonucleic acid (mRNA) encoding a granulocyte colony-stimulating factor (G-CSF) polypeptide lacks at least one destabilizing element.

Provided herein are kits comprising a pharmaceutical formulation described herein and AMD3100 (1,1′-[1,4-phenylene-bis(methylene)]-bis-1,4,8,11-tetraazacyclotetradecane), in one or more containers, and instructions for use thereof.

Further provided herein are pharmaceutical formulations consisting essentially of: (i) an effective amount of a synthetic messenger ribonucleic acid (mRNA) encoding a granulocyte colony-stimulating factor (G-CSF) polypeptide; (ii) a cell penetration agent; and (iii) a pharmaceutically acceptable carrier, wherein i) the mRNA comprises pseudouridine, 5′methyl-cytidine or a combination thereof, or ii) wherein the mRNA does not comprise a substantial amount of a nucleotide or nucleotides selected from the group consisting of uridine, cytidine, and a combination of uridine and cytidine, and wherein the formulation is suitable for repeated intravenous administration to a mammalian subject in need thereof.

Further provided herein are methods to obtain progenitor and/or stem cells from a mammalian subject, comprising the steps of: (a) administering to the subject a composition comprising a synthetic messenger ribonucleic acid (mRNA) encoding a granulocyte colony-stimulating factor (G-CSF) polypeptide in an amount effective to mobilize the progenitor and/or stem cells into the peripheral blood of the subject; followed by (b) harvesting the progenitor and/or stem cells. In certain embodiments, methods are provided wherein the mRNA or a pharmaceutically acceptable salt thereof is administered to the subject in the dosage range of about 1.0 μg/kg-100 mg/kg of body weight. In some embodiments, methods are provided wherein the subject is human. In certain embodiments, compositions are administered to the subject by an intravenous route. In some embodiments, the methods provided herein further comprise the step of administering an effective amount of AMD3100 (1,1′-[1,4-phenylene-bis(methylene)]-bis-1,4,8,11-tetraazacyclotetradecane) to the subject after administering the composition. In some embodiments, the composition is administered at least twice but fewer than ten times prior to administering the AMD3100. In some embodiments, methods are provided, wherein the composition further comprises a synthetic messenger ribonucleic acid (mRNA) encoding i) a macrophage inflammatory protein (MIP) polypeptide or ii) an antibody that prevents EGF binding by EGFR.

Further provided herein are improved regimens for mobilizing hematopoietic stem and progenitor cells in a mammalian subject in need thereof, the regimen comprising an amount of a synthetic messenger ribonucleic acid (mRNA) encoding a G-CSF polypeptide effective to enhance mobilization of hematopoietic stem cells and progenitor cells (HSPC), and an effective amount of AMD3100 (1,1′-[1,4-phenylene-bis(methylene)]-bis-1,4,8,11-tetraazacyclotetradecane).

Further provided herein are pharmaceutical formulations comprising: (i) an effective amount of a synthetic messenger ribonucleic acid (mRNA) encoding an insulin polypeptide; and (ii) a pharmaceutically acceptable carrier, wherein i) the mRNA comprises pseudouridine, 5′methyl-cytidine or a combination thereof, or ii) wherein the mRNA does not comprise a substantial amount of a nucleotide or nucleotides selected from the group consisting of uridine, cytidine, and a combination of uridine and cytidine, and wherein the formulation is suitable for repeated intravenous administration or intramuscular administration to a mammalian subject in need thereof. In certain embodiments, pharmaceutical formulations comprising synthetic messenger ribonucleic acid (mRNA) encoding an insulin polypeptide are provided, wherein the insulin polypeptide comprises: (a) the amino acid sequence of SEQ ID NOs: 15 or 16; (b) an amino acid sequence at least 95% identical to SEQ ID NOs: 15 or 16; (c) a fragment of SEQ ID NOs: 15 or 16; (d) the amino acid sequence encoded by the nucleic acid of SEQ ID NOs: 3 or 4, or SEQ ID NO: 21 or 23; or (e) the amino acid sequence encoded by a nucleic acid at least 95% identical to SEQ ID NOs: 3 or 4, or SEQ ID NO: 21 or 23. In certain embodiments, the pharmaceutical formulations further comprise a lipid-based transfection reagent.

Further provided herein are methods of regulating carbohydrate and lipid metabolism in a mammalian subject in need thereof, comprising the step of: (a) administering to the subject a composition comprising a synthetic messenger ribonucleic acid (mRNA) encoding an insulin polypeptide in an amount effective to regulate carbohydrate and lipid metabolism in the subject.

Provided herein are isolated nucleic acids comprising a translatable region and at least two different nucleoside modifications, wherein the nucleic acid exhibits reduced degradation in a cell into which the nucleic acid is introduced, relative to a corresponding unmodified nucleic acid. In certain embodiments, the nucleic acids comprise RNA, DNA, TNA, GNA or a hybrid thereof. In certain embodiments, the nucleic acids comprise messenger RNA (mRNA). In certain embodiments the mRNA does not substantially induce an innate immune response of a cell into which the mRNA is introduced. In certain embodiments, the mRNA comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In certain embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In other embodiments, the mRNA comprises at least one nucleoside selected from the group consisting of 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In yet other embodiments, the mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In some embodiments, the nucleic acids provided herein comprise a 5′ untranslated region (UTR) and/or a 3′UTR, wherein each of the two different nucleoside modifications are independently present in the 5′UTR and/or 3′UTR. In some embodiments, nucleic acids are provided herein, wherein at least one of the two different nucleoside modifications are present in the translatable region. In some embodiments, nucleic acids are provided herein are capable of binding to at least one polypeptide that prevents or reduces an innate immune response of a cell into which the nucleic acid is introduced.

Further provided herein are isolated polypeptides produced by translation of the mRNAs described herein.

Further provided herein are isolated complexes comprising a conjugate of a protein and a nucleic acid, comprising (i) a mRNA comprising a translatable region and at least two different nucleoside modifications; and (ii) one or more polypeptides bound to the mRNA in an amount effective to prevent or reduce an innate immune response of a cell into which the complex is introduced.

Further provided herein are isolated mRNAs comprising (i) a translatable region, (ii) at least two different nucleoside modifications, and (iii) a degradation domain.

Further provided herein are isolated mRNAs comprising i) a translatable region and ii) at least two different nucleoside modifications, wherein the translatable region encodes a polypeptide variant having an altered activity relative to a reference polypeptide. In certain embodiments, isolated mRNAs are provided, wherein the altered activity comprises an increased activity or wherein the altered activity comprises a decreased activity.

Further provided herein are non-enzymatically synthesized mRNAs comprising at least one nucleoside modification, optionally comprising a translatable region. In certain embodiments, the non-enzymatically synthesized mRNAs comprise at least two different nucleoside modifications. In certain embodiments, the non-enzymatically synthesized mRNAs are substantially not translatable. In certain embodiments, the non-enzymatically synthesized mRNAs are provided in an amount effective as a vaccine when administered to a mammalian subject.

Further provided herein are isolated nucleic acids comprising (i) a translatable region, (ii) at least one nucleoside modification, and (iii) at least one intronic nucleotide sequence capable of being excised from the nucleic acid.

Further provided herein are libraries comprising a plurality of polynucleotides, wherein the polynucleotides individually comprise: (i) a first nucleic acid sequence encoding a polypeptide; (ii) at least one nucleoside modification. In certain embodiments, libraries are provided, wherein the polypeptide comprises an antibody or functional portion thereof. In certain embodiments, libraries are provided, wherein the polynucleotides comprise mRNA. In certain embodiments, libraries are provided, wherein the at least one nucleoside modification is selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

Further provided herein are methods for treating or preventing a symptom of cystic fibrosis in a mammalian subject, comprising contacting a cell of the subject with the nucleic acid of claim 1, wherein the translatable region encodes a Cystic Fibrosis Transmembrane Conductance Regulator (CFTR) polypeptide, under conditions such that an effective amount of the CTFR polypeptide is present in the cell, thereby treating or preventing a symptom of cystic fibrosis in the subject. In certain embodiments, the cell is an epithelial cell, an endothelial cell, or a mesothelial cell. In certain embodiments, the nucleic acid comprises an RNA molecule formulated for administration by inhalation.

Further provided herein are methods for inducing an alteration in cell fate in a mammalian cell, comprising the steps of: (a) providing a precursor cell; and (b) contacting the precursor cell with an effective dose of a cell fate inductive polypeptide encoded by the nucleic acids described herein, under conditions such that an alteration in cell fate is induced.

Further provided herein are methods for enhancing protein product yield in a cell culture process, comprising the steps of: (a) providing a cell culture comprising a plurality of host cells; (b) contacting the cell culture with a composition comprising a nucleic acid comprising a translatable region and at least one nucleoside modification, wherein the nucleic acid exhibits increased protein production efficiency in a cell culture into which the nucleic acid is introduced, relative to a corresponding unmodified nucleic acid. In certain embodiments, methods are provided, wherein the increased protein production efficiency comprises increased cell transfection. In certain embodiments, the increased protein production efficiency comprises increased protein translation from the nucleic acid. In certain embodiments, the increased protein production efficiency comprises decreased nucleic acid degradation. In certain embodiments, the increased protein production efficiency comprises reduced innate immune response of the host cell. In certain embodiments, methods are provided, wherein the cell culture comprises a fed-batch mammalian cell culture process.

Further provided herein are methods for optimizing expression of an engineered protein in a target cell, comprising the steps of: (a) providing a plurality of target cell types; (b) independently contacting with each of the plurality of target cell types an isolated nucleic acid comprising a translatable region encoding an engineered polypeptide and at least one nucleoside modification; and (c) detecting the presence and/or level of the engineered polypeptide in the plurality of target cell types, thereby optimizing expression of an engineered polypeptide in a target cell. In certain embodiments, the engineered polypeptide comprises a post-translational modification. In certain embodiments, the engineered polypeptide comprises a tertiary structure. In certain embodiments, methods are provided, wherein the target cell comprises a mammalian cell line.

Further provided herein are methods for inducing in vivo translation of a recombinant polypeptide in a mammalian subject in need thereof, comprising the step of administering to the subject an effective amount of a composition comprising a nucleic acid comprising: (i) a translatable region encoding the recombinant polypeptide; and (ii) at least one nucleoside modification, under conditions such that the nucleic acid is localized into a cell of the subject and the recombinant polypeptide is capable of being translated in the cell from the nucleic acid. In certain embodiments, the composition comprises mRNA. In certain embodiments, methods are provided, wherein the recombinant polypeptide comprises a functional activity substantially absent in the cell in which the recombinant polypeptide is translated. In certain embodiments, the recombinant polypeptide comprises a polypeptide substantially absent in the cell in the absence of the composition. In certain embodiments, the recombinant polypeptide comprises a polypeptide that antagonizes the activity of an endogenous protein present in, on the surface of, or secreted from the cell. In certain embodiments, the recombinant polypeptide comprises a polypeptide that antagonizes the activity of a biological moiety present in, on the surface of, or secreted from the cell. In certain embodiments, the biological moiety comprises a lipid, a lipoprotein, a nucleic acid, a carbohydrate, or a small molecule toxin. In certain embodiments, the recombinant polypeptide is capable of being secreted from the cell. In certain embodiments, the recombinant polypeptide is capable of being translocated to the plasma membrane of the cell. In certain embodiments, methods are provided, wherein the composition is formulated for administration intramuscularly, transarterially, intraperitoneally, intravenously, intranasally, subcutaneously, endoscopically, transdermally, or intrathecally. In certain embodiments, methods are provided, wherein the composition is formulated for extended release. In certain embodiments, the recombinant polypeptide is capable of being translocated to the plasma membrane of the cell.

Further provided herein are methods for inducing translation of a recombinant polypeptide in a cell population, comprising the step of contacting the cell population with an effective amount of a composition comprising a nucleic acid comprising: (i) a translatable region encoding the recombinant polypeptide; and (ii) at least one nucleoside modification, under conditions such that the nucleic acid is localized into one or more cells of the cell population and the recombinant polypeptide is translated in the cell from the nucleic acid. In certain embodiments, methods are provided, wherein the composition comprises mRNA. In certain embodiments, the composition comprises a cell penetrating compound. In certain embodiments, methods are provided, wherein the step of contacting the cell with the composition is repeated one or more times. In certain embodiments, the step of contacting the cell with the composition is repeated a sufficient number of times such that a predetermined efficiency of protein translation in the cell population.

Further provided herein are methods of reducing the innate immune response of a cell to an exogenous nucleic acid, comprising the steps of: (a) contacting the cell with a first composition comprising a first dose of a first exogenous nucleic acid comprising a translatable region and at least one nucleoside modification; (b) determining the level of the innate immune response of the cell to the first exogenous nucleic acid; (c) contacting the cell with a second composition comprising either: (i) a second dose of the first exogenous nucleic acid, wherein the second dose contains a lesser amount of the first exogenous nucleic acid as compared to the first dose; or (ii) a first dose of a second exogenous nucleic acid, thereby reducing the innate immune response of the cell. In certain embodiments, methods are provided, wherein the step of contacting the cell with the first composition and/or the second composition is repeated one or more times. In certain embodiments, a predetermined efficiency of protein translation in the cell is achieved.

Further provided herein are methods for silencing gene expression in a cell, comprising the step of contacting the cell with a nucleic acid comprising: (i) a translatable region encoding a polypeptide capable of directing sequence-specific histone H3 methylation, under conditions such that the polypeptide is translated and reduces gene transcription of a target gene; and (ii) and at least one nucleoside modification. In certain embodiments, the cell is present in a mammalian subject. In certain embodiments, the target gene is a mutated Janus Kinase-2 family member and the mammalian subject suffers from a myeloproliferative disease.

Further provided herein are methods of reducing cellular differentiation in a target cell population, comprising the step of contacting the target cell population comprising a precursor cell with a composition comprising an effective amount of a nucleic acid comprising a translatable region encoding a polypeptide and at least one modified nucleoside, under conditions such that the polypeptide is translated and reduces the differentiation of the precursor cell. In certain embodiments, the target cell population comprises injured tissue. In certain embodiments, the target cell population comprises tissue affected by a surgical procedure. In certain embodiments, the precursor cell is selected from the group consisting of a stromal precursor cell, a neural precursor cell, and a mesenchymal precursor cell.

Further provided herein are isolated nucleic acids comprising a noncoding region and at least one nucleoside modification that reduces an innate immune response of a cell into which the nucleic acid is introduced, wherein the nucleic acid sequesters one or more translational machinery components. In certain embodiments, the isolated nucleic acids comprising a noncoding region and at least one nucleoside modification described herein are provided in an amount effective to reduce protein expression in the cell. In certain embodiments, the translational machinery component is a ribosomal protein or a transfer RNA (tRNA). In certain embodiments, the nucleic acid comprises a small nucleolar RNA (sno-RNA), micro RNA (miRNA), small interfering RNA (siRNA) or Piwi-interacting RNA (piRNA).

Further provided herein are isolated nucleic acids comprising (i) a first translatable region, (ii) at least one nucleoside modification, and (iii) an internal ribosome entry site (IRES). In certain embodiments, the IRES is obtained from a picornavirus, a pest virus, a polio virus, an encephalomyocarditis virus, a foot-and-mouth disease virus, a hepatitis C virus, a classical swine fever virus, a murine leukemia virus, a simian immune deficiency virus or a cricket paralysis virus. In certain embodiments, the isolated nucleic acids further comprise a second translatable region. In certain embodiments, the isolated nucleic acids further comprise a Kozak sequence.

Further provided herein are methods of antagonizing a biological pathway in a cell, comprising the step of contacting the cell with an effective amount of a composition comprising a nucleic acid comprising: (i) a translatable region encoding a recombinant polypeptide; and (ii) at least one nucleoside modification, under conditions such that the nucleic acid is localized into the cell and the recombinant polypeptide is capable of being translated in the cell from the nucleic acid, wherein the recombinant polypeptide inhibits the activity of a polypeptide functional in the biological pathway. In certain embodiments, methods are provided, wherein the biological pathway is defective in an autoimmune or inflammatory disorder selected from the group consisting of multiple sclerosis, rheumatoid arthritis, psoriasis, lupus erythematosus, ankylosing spondylitis and Crohn's disease.

Further provided herein are methods of agonizing a biological pathway in a cell, comprising the step of contacting the cell with an effective amount of a composition comprising a nucleic acid comprising: (i) a translatable region encoding a recombinant polypeptide; and (ii) at least one nucleoside modification, under conditions such that the nucleic acid is localized into the cell and the recombinant polypeptide is capable of being translated in the cell from the nucleic acid, wherein the recombinant polypeptide induces the activity of a polypeptide functional in the biological pathway. In certain embodiments, the agonized biological pathway modulates cell fate determination. In certain embodiments, the biological pathway is reversibly agonized.

Further provided herein are methods for enhancing nucleic acid delivery into a cell population, comprising the steps of: (a) providing a cell culture comprising a plurality of host cells; (b) contacting the cell population with a composition comprising an enhanced nucleic acid comprising a translatable region and at least one nucleoside modification, wherein the enhanced nucleic acid exhibits enhanced retention in the cell population, relative to a corresponding unmodified nucleic acid. In certain embodiments, methods are provided, wherein the retention of the enhanced nucleic acid is at least about 50% greater than the retention of the unmodified nucleic acid. In some embodiments, the retention of the enhanced nucleic acid is at least about 100% greater than the retention of the unmodified nucleic acid. In other embodiments, the retention of the enhanced nucleic acid is at least about 200% greater than the retention of the unmodified nucleic acid. In certain embodiments, methods are provided, wherein the step of contacting the cell with the composition is repeated one or more times.

Further provided herein are methods of nucleic acid co-delivery into a cell population, comprising the steps of: (a) providing a cell culture comprising a plurality of host cells; (b) contacting the cell population with a composition comprising: (i) a first enhanced nucleic acid comprising a translatable region and at least one nucleoside modification; and (ii) a first unmodified nucleic acid, wherein the composition does not substantially induce an innate immune response of the cell population.

Further provided herein are methods of nucleic acid delivery into a cell population, comprising the steps of: (a) providing a cell culture comprising a plurality of host cells; (b) contacting the cell population with a first composition comprising: (i) a first enhanced nucleic acid comprising a translatable region and at least one nucleoside modification; and (ii) a first unmodified nucleic acid, wherein the composition does not substantially induce an innate immune response of the cell population; and (c) contacting the cell population with a second composition comprising a first unmodified nucleic acid.

Patients who have had chemotherapy and/or radiation therapy may require infusions of progenitor and/or stem cells to restore their immune system. To obtain the stem cells, they need to be mobilized or moved into the peripheral blood. A fast and durable recovery of a patient's immune system is usually achieved if a sufficient number of stem cells is available for transplantation. Stem cell transplantation can be either allogenic (cells are transplanted from a healthy donor, e.g. a sibling), or autologous (cells are collected from the patient and reinfused after chemotherapy). Following chemotherapy, the stored stem cells can be transplanted into the patient, through an intravenous infusion. Patients may be given antibiotics and/or blood transfusions to prevent infection while their immune systems are recovering. Once in the bloodstream the stem cells migrate back into the bone marrow. Over a period of one to five weeks, these stem cells can increase in number and develop into various types of cells including neutrophils. Current strategies of mobilizing bone marrow progenitor and/or stem cells into the blood stream employ G-CSF, e.g. as described in U.S. Pat. No. 5,582,823. Agents that may further increase circulating white blood cells and progenitor cells and that can be used in combination with G-CSF include AMD3100, granulocyte-macrophage colony stimulating factor (GM-CSF), Interleukin-1 (IL-I), Interleukin-3 (IL-3), Interleukin-8 (IL-8), PIXY-321 (GM-CSF/IL-3 fusion protein), macrophage inflammatory protein, stem cell factor (SCF), thrombopoietin, flt3, myelopoietin, anti-VLA-4 antibody, anti-VCAM-1 and growth related oncogene (GRO). PCT publication No. WO/2008/019371; Dale D et al. Am. J. of Hematol. 57:7-15 (1998); Rosenfeld C et al. Bone Marrow Transplantation 17:179-183 (1997); Pruijt J et al. Cur. Op. in Hematol. 6:152-158 (1999); Broxmeyer H et al. Exp. Hematol. 23:335-340 (1995); Broxmeyer H et al. Blood Cells, Molecules and Diseases 24:14-30 (1998); Glaspy J et al. Cancer Chemother. Pharmacol. 38 (suppl): S53-S57 (1996); Vadhan-Raj S et al. Ann. Intern. Med. 126:673-681 (1997); King A et al. Blood 97:1534-1542 (2001); Glaspy J et al. Blood 90:2939-2951 (1997); and Papayannopoulou T et al. PNAS 92:9647-9651 (1995). AMD3100 (l,l[1,4-phenylene-bis(methylene)]-bis-1,4,8,ll-tetraazacyclotetradecane) has been shown to mobilize progenitor/stem cells to permit their harvest (PCT publication No. WO 03/011277) and to elevate white blood cell count (WO 00/458140).

Provided herein are improved regimens for mobilizing hematopoietic stem and progenitor cells in a mammalian subject in need thereof, the regimen comprising an amount of a modified mRNA (mmRNA) encoding a G-CSF polypeptide effective to enhance mobilization of hematopoietic stem cells and progenitor cells (HSPC), and an effective amount of AMD3100.

Further provided herein are methods to obtain progenitor and/or stem cells from a mammalian subject, comprising the steps of: (a) administering to the subject a composition comprising a modified mRNA encoding G-CSF polypeptide in an amount effective to mobilize the progenitor and/or stem cells into the peripheral blood of the subject; (b) administering an effective amount of AMD3100 to the subject after administering the composition; followed by (c) harvesting the progenitor and/or stem cells. In some embodiments, the composition is administered at least twice but fewer than ten times prior to administering the AMD3100.

Further provided herein are kits comprising a pharmaceutical formulation comprising a modified mRNA encoding G-CSF polypeptide and AMD3100 in one or more containers, and instructions for use thereof.

### BRIEF DESCRIPTION OF THE DRAWINGS

FIG. 1A-FIG. 1C depict graphs showing human G-CSF (FIG. 1A), α-MSH (FIG. 1B) and mouse insulin (FIG. 1C) secretion into the media (pg/ml) measured by ELISA following transfection of human keratinocytes with various amounts of modified mRNA.

FIG. 2 depicts a photograph showing human G-CSF expression in human cells following transfection with modified mRNA as measured by immunofluorescence.

FIG. 3 depicts a photograph showing mouse insulin expression in human cells following transfection with modified mRNA as measured by immunofluorescence.

FIG. 4-1, FIG. 4-2 and FIG. 4-3 depict bar graphs of complete blood count and granulocyte lineage cell types of a mouse following intravenous injection of modified huG-CSF mRNA (FIG. 4-1 for Day 1, FIG. 4-2 for Day 5, and FIG. 4-3 for Day 8).

FIG. 5-1, FIG. 5-2 and FIG. 5-3 depict bar graphs of erythrocyte and lymphocyte lineage cell types of a mouse following intravenous injection of modified huG-CSF mRNA (FIG. 5-1 for Day 1, FIG. 5-2 for Day 5, and FIG. 5-3 for Day 8).

### DETAILED DESCRIPTION OF THE INVENTION

In general, exogenous nucleic acids, particularly viral nucleic acids, introduced into cells induce an innate immune response, resulting in interferon (IFN) production and cell death. However, it is of great interest for therapeutics, diagnostics, reagents and for biological assays to deliver a nucleic acid, e.g., a ribonucleic acid (RNA) inside a cell, either in vivo or ex vivo, such as to cause intracellular translation of the nucleic acid and production of the encoded protein. Of particular importance is the delivery and function of a non-integrative nucleic acid, as nucleic acids characterized by integration into a target cell are generally imprecise in their expression levels, deleteriously transferable to progeny and neighbor cells, and suffer from the substantial risk of mutation. Provided herein in part are nucleic acids encoding useful polypeptides capable of modulating a cell's function and/or activity, and methods of making and using these nucleic acids and polypeptides. As described herein, these nucleic acids are capable of reducing the innate immune activity of a population of cells into which they are introduced, thus increasing the efficiency of protein production in that cell population. Further, one or more additional advantageous activities and/or properties of the nucleic acids and proteins of the invention are described.

Provided herein are modified nucleic acids encoding the precursors of melanocyte-stimulating hormone (MSH), insulin, and granulocyte colony-stimulating factor (G-CSF), or partially or fully processed forms of these precursors. In certain embodiments, the modified nucleic acids comprise mRNA. In particular embodiments, the modified mRNA (mmRNA) is derived from cDNA. In certain embodiments, the mmRNA comprises at least two nucleoside modifications. In certain embodiments, these nucleoside modifications are 5-methylcytosin and pseudouridine. In some embodiments, around 25%, around 50%, around 75%, or up to and including 100% of cytosine and uridine nucleotides of the modified nucleic acid are modified nucleotides. In certain embodiments, the mmRNA comprises a 5′ cap structure and a 3′ poly-A tail. In specific embodiments, the 5′ cap structure is a Cap 1 structure. In specific embodiments, the poly-A tail comprises at least 10, 20, 30, 40, 50, 60, 70, 80, 90, 100, 110, 120, 130, 140, or 150 nucleotides.

Further, provided herein are compositions comprising the modified nucleic acids described herein. In certain embodiments, the compositions further comprise a pharmaceutically acceptable carrier. In certain embodiments, the carrier is formulated for systemic or local administration. In certain embodiments, the administration is oral or topical. In certain embodiments, the compositions comprise naked modified nucleic acids. In other embodiments, the modified nucleic acids are complexed or encapsulated. For example, the modified nucleic acids may be complexed in liposomal form or may be encapsulated in a nanoparticle. In certain embodiments, the modified nucleic acids, the complex or the nanoparticle further comprise one or more targeting moieties. These moieties can be used to target delivery in vivo to certain organs, tissues or cells.

Provided herein are methods of treating a subject having or being suspected of having a disease, the methods comprising administering to a subject in need of such treatment a composition described herein in an amount sufficient to treat the disease. In specific embodiments, the disease is associated with one or more cellular and/or molecular changes affecting, for example, the level, activity, and/or localization of precursors of melanocyte-stimulating hormone (MSH), insulin, and granulocyte colony-stimulating factor (G-CSF), or a partially or fully processed form of these precursors. Cellular and/or molecular changes may affect transcription, translation, posttranslational modification, processing, folding, intra- and/or extracellular trafficking, intra- and/or extracellular stability/turnover, and/or signaling of one or more molecules associated with MSH, G-CSF or insulin activity. In certain embodiments, activities associated with MSH, G-CSF or insulin are compromised, e.g. 90%, 80%, 70%, 60%, 50%, 40%, 30%, 20%, 10%, 5% or less of wild-type activity. For signaling molecules, such as hormones, whether they are autocrine or paracrine, the one or more defects resulting in reduced activity may be exhibited by the cell in which MSH, G-CSF or insulin are generated and/or may be exhibited by the cell on which MSH, G-CSF or insulin act. In certain embodiments, the methods of treating a subject having or being suspected of having a disease comprise administering to the subject in need of such treatment a composition comprising a modified nucleic acid described herein in an amount sufficient to modulate one or more activities associated with MSH, G-CSF or insulin to treat the disease.

A major drawback of many current treatments for diseases described herein is the necessity to produce MSH, insulin, and G-CSF as polypeptides. Polypeptides are ordinarily expressed in and isolated from mammalian or bacterial cultures. Bacterial cultures and many cancer-derived cell culture systems do not faithfully recapitulate post-translational modifications, such as e.g. glycosylation (G-CSF) and amidation (MSH) and protein precursors may not be fully processed. In some instances, the lack of posttranslational modification and processing influences the activity of the final protein product, its localization and/or its target specificity. For example, pro-opiomelanocortin (POMC) if not fully processed may give rise to adrenocorticotropic hormone (ACTH) which is MC2R-specific, while MC1R has the highest affinity to α-MSH, which is identical to the first 13 amino acids at the amino terminal of ACTH. Precursors and final cleavage products can have different physiological effects. In type 2 diabetes, an elevated proinsulin-to-insulin ratio in blood circulation has been detected, likely as a consequence of compromised proteolytic processing of insulin precursor. For production of recombinant proteins, the polypeptide product that is effective for a particular treatment must usually be predetermined because the proteins if administered do not undergo any additional processing. Any modification that is vital for activity must also be present on the recombinant protein because they will not be added by the host when the recombinant proteins are administered. Recombinant protein production and purification is expensive and labor intensive. Protein expression host systems may harbor pathogens (e.g. viruses) that may contaminate the purified product. Proteins and particularly protein modifications are inherently unstable und require specific storage conditions and generally have a short shelf life. To be efficacious, recombinant proteins must be further modified, particularly by pegylation to avoid rapid degradation in vivo. For example, Filgrastim was readily degraded in vivo and made repeated daily injections necessary during the course of chemotherapy. Only site-specific pegylation at the N-terminus of G-CSF (PEG-Filgrastim, U.S. Pat. Nos. 5,824,784 and 7,090,835) made the product stable enough to be given at less frequent intervals. Still, site-specific pegylation remains difficult because it can lead to loss of activity, loss of target specificity and/or protein aggregation. Veronese et al. Bioconjugate Chem. 18:1824-1830 (2007).

The modified mRNA molecules described herein do not share these problems. In comparison to recombinant proteins, they exhibit increased stability for shipping, handling and storage, are easy to mass produce, and when translated from the modified mRNA, the polypeptide can undergo an array of cell- and/or tissue-specific posttranslational processing, folding and modification.

### Melanocyte-Stimulating Hormones (MSH).

The melanocyte-stimulating hormones (MSH) are peptide hormones primarily produced by cells in the intermediate lobe of the pituitary gland. MSH belongs to a group called the melanocortins. This group includes and adrenocorticotropic hormone (ACTH), α-MSH, β-MSH and γ-MSH. These peptides are all cleavage products of a large precursor peptide called pro-opiomelanocortin (POMC). α-MSH is identical to the first 13 amino acids at the amino terminal of ACTH. Proteolytic cleavage has been identified in several cell types such as melanocytes, keratinocytes, epithelial cells, B cells, natural killer cells and subsets of T cells. MSH is an agonist of and exerts its biological function through five different melanocortin-receptors (MCRs). MCRs are G-protein coupled receptors with seven-transmembrane domains that signal by activating adenylate cyclase resulting in an increase of intracellular cAMP. α-MSH is largely non-selective, although MC1R has the highest affinity to α-MSH, while ACTH is MC2R-specific and γ-MSH is MC3R specific. MCRs are inhibited by antagonists agouti signaling peptide and agouti-related peptide.

The amino acid sequences of melanocyte-stimulating hormones are set forth below:
α-MSH: Ser-Tyr-Ser-Met-Glu-His-Phe-Arg-Trp-Gly-Lys-Pro-Val (SEQ ID NO: 10)
β-MSH Ala-Glu-Lys-Lys-Asp-Glu-Gly-Pro-Tyr-Arg-Met-Glu-His-Phe-Arg-Trp-Gly-(human): Ser-Pro-Pro-Lys-Asp (SEQ ID NO: 11)
γ-MSH: Tyr-Val-Met-Gly-His-Phe-Arg-Trp-Asp-Arg-Phe-Gly (SEQ ID NO: 12)

Synthetic analogs of α-MSH have been developed for human use, e.g. afamelanotide (melanotan-1, SCENESSE® or CUV1647) (Clinuvel Pharmaceuticals, Australia) and bremelanotide (PT-141) (Palatin Technologies, NJ).

Conditions affecting cortisol levels (e.g. Cushing's syndrome and primary Addison's) influence ACTH production from POMC and can also increase MSH levels which can cause hyper-pigmentation. Subjects who do not tan well may express variant MCRs that are refractory to MSH in the blood. Subjects with red hair have a predominance of red phaeomelanin in hair and skin and/or a reduced ability to produce black eumelanin. Eumelanin is photoprotective whereas phaeomelanin can generate free radicals in response to UV radiation and may contribute to UV-induced skin damage, which is one reason why red haired subjects fail to tan and are at risk from UV-radiation. In mammals the relative proportions of phaeomelanin and eumelanin are regulated by MSH via MC1R. A variant MC1R is present in over 80% of subjects with red hair and/or fair skin who tan poorly which can be found in only 4% of subjects who show a good tanning response. Valverde P et al. Nat Genet. 11:328-30 (1995). Certain mutations in the MC1R gene are strongly associated with increased melanoma incidence by sensitizing melanocytes to the cytotoxic effects of UV irradiation. Abdel-Malek Z A et al. Photochem Photobiol 84:501-508 (2008). The MC1R coding sequence is highly polymorphic and certain allelic variants are associated with pigmentation phenotypes and risk factors for melanoma and non-melanoma skin cancer development. Wong T H et al. Peptides 26:1965-1971 (2005), Sturm R A Melanoma Res 12:405-416 (2002). Sánchez-Laorden B L et al. J Biol Chem 282:3241-3251 (2007). Four alleles have a strong association with the red hair/fair skin phenotype: D84E, R151C, R160W, and D294H. V60L, V92M, and R163Q are weakly associated. Loss of function alleles, such as R151C, R160W, R142H, and D294H, which modulate α-MSH binding and/or adenylyl cyclase activation, lead to increased sensitivity to UV radiation and an increased risk for developing skin cancer, e.g. melanoma. Makova K et al. Peptides 26:1901-1908 (2005), Sturm R A et al. Gene 277:49-62 (2001), Scott M C et al. J Cell Sci 115:2349-2355 (2002). UV irradiation is thought to up-regulate α-MSH/MC1R signaling resulting in increased pigmentation of the skin and enhanced levels of cytotoxic CD8⁺ T cell surveillance to protect against skin cancer development. Loss-of-function mutations in the MC1R gene might prevent the induction of tumor protection.

α-MSH is responsible for tanning in humans and α-MSH polypeptide analogs are being investigated for photo-protection in subjects with various indications, such as erythropoietic protoporphyria (EPP), polymorphous light eruption (PLE), actinic keratosis (solar keratosis) and squamous cell carcinoma. MSH agonist effects are thought to be mediated by MC1R. α-MSH is responsible for pigmentation by stimulating the production and release of melanin in melanocytes of the skin and hair. When ultraviolet light contacts keratinocytes, p53 is activated which in turn activates transcription of the POMC gene. POMC protein cleavage produces α-MSH which is secreted from the cells and stimulates melanocytes in a paracrine manner to synthesize melanin. The melanin is secreted by the melanocytes and taken up by the skin cells. ACTH is secreted into the blood and may help reduce skin inflammation by stimulating the release of glucocorticoids from the adrenal cortex.

α-MSH plays a role in MHC class I-restricted cytotoxicity. Treatment of skin cancer with α-MSH might ameliorate disease or improve anti-tumoral immune responses. It was recently demonstrated that MC1R is expressed in murine and human CD8⁺ T cells and that α-MSH/MC1R-mediated signaling induces up-regulation of the expression of cytotoxic genes and enhances the cytolytic activity in tumor-specific CD8⁺ T cells. Loser K et al. PLOS ONE 5 (2): e8958. α-MSH has an inhibitory effect on melanoma proliferation and metastasis formation and its anti-inflammatory properties may also affect melanoma progression. Lunec J et al. Melanoma Res 2:5-12 (1992), Gehlsen K R et al. Pigment Cell Res 5:219-223 (1992), Eves P et al. Br J Cancer 89:2004-2015 (2003).

Provided herein are methods of treating a subject having a skin cancer, the methods comprising administering to a subject in need of such treatment a composition comprising a modified nucleic acid encoding a MSH precursor or a partially or fully processed form thereof in an amount sufficient to treat the skin cancer. In certain embodiments, the skin cancer is a melanoma or a squamous cell carcinoma. In certain embodiments, the treatment is local. In certain embodiments, the treatment is topical. In certain embodiments, cells, e.g. dendritic cells, autologously derived from the recipient subject are stimulated ex vivo using a modified nucleic acid encoding a MSH precursor or a partially or fully processed form. These cells may be re-administered to the subject having the skin cancer to treat the condition.

Provided herein are methods of preventing the development of skin cancer or reducing the likelihood that a subject develops skin cancer. The methods comprise administering to a subject in need of such prevention a composition comprising a modified nucleic acid encoding a MSH precursor or a partially or fully processed form thereof in an amount sufficient to prevent the development of skin cancer or to reduce the likelihood that a subject develops skin cancer. In certain embodiments, the subject in need of prevention is a subject exposed to sun light. In certain embodiments, the skin cancer is a melanoma or a squamous cell carcinoma. In certain embodiments, the administration is local. In certain embodiments, the administration is topical. For example, a composition comprising a modified nucleic acid encoding a MSH precursor or a partially or fully processed form thereof may comprise suntan lotion. In certain embodiments, the composition is applied as a prophylactic, before exposure to light, e.g. sun light.

Provided herein are methods of preventing the development of a condition associated with photo-sensitivity or reducing the likelihood that a subject develops the condition and/or displays one or more symptoms of the condition. The methods comprise administering to a subject in need of such prevention a composition comprising a modified nucleic acid encoding a MSH precursor or a partially or fully processed form thereof in an amount sufficient to prevent the development of a condition associated with photo-sensitivity or reducing the likelihood that the subject develops the condition and/or displays one or more symptoms of the condition. In certain embodiments, the condition associated with photo-sensitivity is erythropoietic protoporphyria (EPP), polymorphous light eruption (PLE), or actinic keratosis (solar keratosis). In certain embodiments, the administration is local. In certain embodiments, the administration is topical. For example, a composition comprising a modified nucleic acid encoding a MSH precursor or a partially or fully processed form thereof may comprise suntan lotion. In certain embodiments, the composition is applied as a prophylactic, before exposure to a light source, e.g. an interior or exterior light source. In specific embodiments, the exterior light source is the sun.

Provided herein are methods of increasing or accelerating the process of tanning in a subject. The methods comprise administering to a subject wishing to increase or accelerate the process of tanning a composition comprising a modified nucleic acid encoding a MSH precursor or a partially or fully processed form thereof in an amount sufficient to increase or accelerate the process of tanning. In certain embodiments, the treatment is local. In certain embodiments, the treatment is topical. For example, a composition comprising a modified nucleic acid encoding a MSH precursor or a partially or fully processed form thereof may comprise a lotion for use as a cosmetic.

In certain embodiments, the subject exhibits wild-type MSH/MCR signaling. In other embodiments, the subject exhibits impaired MSH/MCR signaling. The impairment may be reduced signaling. For example, impaired signaling may be caused by reduced intra- or extracellular levels of MSH or a lack of MSH. Impaired signaling may be caused by dysfunctional MC receptor, e.g. as a result of a mutation in a gene encoding an MCR. MSH/MCR signaling may be impaired by at least 5%, 10%, 20%, 30%, 40%, 50%, 60%, 70%, 80%, 90%, 95%, or 99% compared to wild-type MSH/MCR signaling. The resulting impairment may lead to a reduction in melanin production, a change in the relative proportions of phaeomelanin and eumelanin toward phaeomelanin, a reduction in inflammatory responses, a reduction in general immune surveillance and/or a reduction specifically in surveillance by cytotoxic CD8⁺ T cell. In some embodiments, compositions comprising a modified nucleic acid encoding a MSH precursor or a partially or fully processed form thereof are administered to modulate MSH/MCR signaling. In certain embodiments, impaired MSH/MCR signaling and/or MSH activity is partially or fully restored compared to wildtype. In certain embodiments, impaired MSH/MCR signaling and/or MSH activity is restored to 5%, 10%, 20%, 30%, 40%, 50%, 60%, 70%, 80%, 90%, 95%, 99% or 100% of wild-type MSH/MCR signaling and/or MSH activity. In other embodiments, restored MSH/MCR signaling and/or MSH activity exceeds that of wildtype activity by 5%, 10%, 20%, 30%, 40%, 50%, 60%, 70%, 80%, 90%, 95%, 100%, 200%, 300%, 400%, 500%, 600%, 700%, 800%, 900%, or 1000%. In certain embodiments, MSH/MCR signaling and/or MSH activity is not impaired in a subject, and compositions comprising a modified nucleic acid encoding a MSH precursor or a partially or fully processed form thereof are administered to increase MSH/MCR signaling above wildtype levels. In some embodiments, MSH/MCR signaling and/or MSH activity exceeds that of wildtype activity by 5%, 10%, 20%, 30%, 40%, 50%, 60%, 70%, 80%, 90%, 95%, 100%, 200%, 300%, 400%, 500%, 600%, 700%, 800%, 900%, or 1000%. In certain embodiments, increased MSH/MCR signaling and/or MSH activity may lead to increased melanin production, increased accumulation of eumelanin, an increased inflammatory, an increased immune response and/or an increase in surveillance by (and/or number of) cytotoxic CD8⁺ T cell.

MSH signaling in the brain has been shown to affect sexual arousal and appetite. MC1R, MC3R and MC4R are widely expressed in the brain, and are also thought to be responsible for effects on mood and cognition.

α-MSH analogs (e.g. melanotan II) are being investigated for their aphrodisiac effects. Enhanced libido and penile erections were noted in male test subject and enhanced arousal in female test subjects. These effects are thought to be mediated by actions in the hypothalamus on neurons that express MSH receptors MC3R and MC4R.

Provided herein are methods of treating a subject suffering from impotence, the methods comprising administering to a subject in need of such treatment a composition comprising a modified nucleic acid encoding a MSH precursor or a partially or fully processed form thereof in an amount sufficient to treat impotence. In certain embodiments, the treatment is targeted to the hypothalamus. In other embodiments, the treatment is systemic. In certain embodiments, penile erections are restored partially or fully.

Provided herein are methods of enhancing libido, penile erections and/or sexual arousal in a subject, the methods comprising administering to a subject in need of such enhancement a composition comprising a modified nucleic acid encoding a MSH precursor or a partially or fully processed form thereof in an amount sufficient to provide the enhancement. In some embodiments, the treatment is systemic. In certain embodiments, the treatment is targeted to the hypothalamus. In certain embodiments, the subject is a male. In other embodiments, the subject is a female.

α-MSH is found in the brain where it is thought to act to suppress appetite. Some cases of extreme obesity have been traced to mutations in the brain receptor for α-MSH. Defects in MCR4 may cause autosomal dominant obesity, accounting for an estimated 5-6% of all cases of early-onset obesity. Farooqi I S et al. N Engl J Med 348:1085-95 (2003), Branson R et al. N Engl J Med 348:1096-103 (2003), Vaisse C et al. J Clin Invest 106:253-62 (2000). Several different monogenic causes of childhood obesity have been identified of which binding of α-MSH to MC4R appears critical. MC4R is expressed in the hypothalamus. Stimulation of MC4R by α-MSH binding triggers the activation of anorexigenic signals, which, are thought to reduce food intake by creating the perception of satiety. Children with MC4R-related obesity are largely unable to experience a sense of satiety. In vitro studies have shown that certain obesity-linked MC4R mutations render the receptor dys- or non-functional thus preventing the activation of anorexigenic signals in response to α-MSH binding. Lubrano-Berthelier C et al. Hum Mol Genet 12:145-53 (2003); Yeo G S et al. Hum Mol Genet 12:561-74 (2003), Nijenhuis W A J Biol Chem 278:22939-45 (2003). MC4R-associated early onset obesity symptoms include binge eating behavior, severe hyperinsulinemia, an increase in bone minerals, a higher linear growth velocity, and an earlier than normal onset of puberty. Early onset obesity first occurs in children under ten years of age. Subjects who are heterozygous for mutations in the mc4r gene exhibit a less severe phenotype than subjects who are homozygous for mc4r gene mutations. In heterozygotes, expression of the obesity phenotype appears to be due to haploinsufficiency and insufficient levels of wildtype MC4R protein are expressed from the normal gene copy. Penetrance of the mutation varies within and between families and not all heterozygous individuals carrying an obesity-associated MC4R mutation are obese. Within families, female carriers of obesity-linked MC4R mutations are often more severely affected than males with the same mutation. Hinney A et al. J Clin Endocrinol Metab 88:4258-67 (2003). A majority of subjects with MC4R-associated obesity are heterozygous carriers. Therapy for MC4R-associated obesity may be targeted to increase stimulation of the remaining wildtype MC4R protein to compensate for the loss-of-function in the mutated receptor. Mutations within POMC genes are also associated with early-onset obesity. Patients have been identified carrying heterozygous mutations in exon 3 (G7013T, C7133delta) which interfere with appropriate synthesis of ACTH and α-MSH, or carrying homozygous mutations in exon 2 (C3804A) which abolishes POMC translation. Krude H et al. Nat Genet 19:155-7 (1998).

Medical and behavioral interventions, such as lifestyle modifications, pharmacotherapy and bariatric surgery, as treatment for obesity in childhood remain scarce and largely ineffective. Lifestyle modifications, e.g. changes to dietary and exercise habits, are effective in less than 5% of obese subjects. Pharmacotherapy is not routinely proposed as a treatment for childhood obesity and the long-term safety and efficacy of anti-obesity drugs (e.g. sibutramine (Meridia®) and orlistat (Xenical®)) have not been determined in children or adolescents. Invasive bariatric surgery to combat morbid obesity in children is not universally accepted as a tool for subjects in childhood or adolescence. Choquet et al. Genome Medicine 2010 2:36.

Provided herein, are methods of treating a subject having early onset obesity, the methods comprising administering to a subject in need of such treatment a composition comprising a modified nucleic acid encoding a MSH precursor or a partially or fully processed form thereof in an amount sufficient to treat early onset obesity. In certain embodiments, the subject is a child or an adolescent. In specific embodiments, the child or an adolescent is female. In certain embodiments, the treatment is systemic. In certain embodiments, the treatment is targeted to the hypothalamus.

Further provided herein, are methods of preventing the development of early onset obesity in a subject suspected of developing early onset obesity, the methods comprising administering to a subject in need of such treatment a composition comprising a modified nucleic acid encoding a MSH precursor or a partially or fully processed form thereof in an amount sufficient to prevent the development of early onset obesity. In certain embodiments, the subject is a child or an adolescent. In specific embodiments, the child or an adolescent is female. In certain embodiments, the treatment is systemic. In certain embodiments, the treatment is targeted to the hypothalamus.

Further provided herein, are methods of delaying the onset of early onset obesity in a subject suspected of developing early onset obesity, the methods comprising administering to a subject in need of such treatment a composition comprising a modified nucleic acid encoding a MSH precursor or a partially or fully processed form thereof in an amount sufficient to delay the onset of early onset obesity. In certain embodiments, the subject is a child or an adolescent. In specific embodiments, the child or an adolescent is female. In certain embodiments, the treatment is systemic. In certain embodiments, the treatment is targeted to the hypothalamus. In certain embodiments, the subject suspected of developing early onset obesity was genetically tested for the presence of one or more mutations in the mc4r gene(s) and/or pomc gene(s). In certain embodiments, testing comprises DNA sequencing of one or more regions of the subject's mc4r gene(s) and/or pomc gene(s). In certain embodiments, DNA is obtained from leukocytes derived from a sample of the subject's blood.

α-MSH exhibits immunomodulatory effects in vivo. Mouse model have shown that systemic as well as epicutaneous application of α-MSH suppressed the sensitization and elicitation phase of contact allergy and induced a hapten-specific tolerance. Grabbe S et al. J Immunol 156:473-478 (1996). Systemic administration of α-MSH inhibits the development of experimentally induced inflammatory bowel disease (IBD, colitis), experimental autoimmune encephalomyelitis and allergic airway inflammation in mice as well as experimentally induced arthritis and experimental uveitis in rats. Rajora N et al Peptides 18:381-385 (1997), Lipton J M et al. Ann N Y Acad Sci 741:137-148 (1994), Han D et al. Gene Ther 14:383-395 (2007), Raap U et al. J Immunol 171:353-359 (2003) Nishida T et al. Int Immunopharmacol 4:1059-1066 (2004).

Provided herein, are methods of providing immunomodulatory effects to a subject having an inflammatory disease, the methods comprising administering to a subject in need of such immunomodulation a composition comprising a modified nucleic acid encoding a MSH precursor or a partially or fully processed form thereof in an amount sufficient to provide an immunomodulatory effect. In certain embodiments, the immunomodulatory effect suppressed the sensitization and/or elicitation phase of contact allergy. In certain embodiments, the immunomodulatory effect inhibits the development of, delays the onset of, or reduces the severity of an inflammatory disease or condition. In certain embodiments, the inflammatory disease or condition is inflammatory bowel disease (IBD, e.g. colitis), autoimmune encephalomyelitis, allergic airway inflammation, arthritis or uveitis. In some embodiments, administration is systemic or epicutaneous. In some embodiments, administration is oral, topical, or pulmonary.

Mucin hypersecretion is a clinical feature of several respiratory diseases such as asthma, cystic fibrosis, nasal allergy, rhinitis, and sinusitis. α-MSH may be used to decrease mucin overproduction initiated by NF-κB activation, because it inhibits NF-κB activation induced by pro-inflammatory cytokines such as TNF-α. α-MSH may be used to alleviate the symptoms of these diseases. Lee S N Am J Respir Cell Mol Biol 44:716-724 (2011).

Provided herein, are methods of modulating NF-κB activation, the methods comprising administering to a subject in need of such modulation a composition comprising a modified nucleic acid encoding a MSH precursor or a partially or fully processed form thereof in an amount sufficient to modulate NF-κB activation. In certain embodiments, modulating NF-κB activation is inhibiting NF-κB activation. In certain embodiments, NF-κB activation is associated with mucine hypersecretion.

Further provided herein, are methods of treating a subject having a disease or condition associated with mucin hypersecretion, the methods comprising administering to a subject in need of such treatment a composition comprising a modified nucleic acid encoding a MSH precursor or a partially or fully processed form thereof in an amount sufficient to treat disease or condition associated with mucin hypersecretion. In some embodiments, mucin hypersecretion is associated with a respiratory disease. In some embodiments, the respiratory disease is asthma, cystic fibrosis, nasal allergy, rhinitis, or sinusitis. In some embodiments, administration is systemic. In certain embodiments, the administered composition is targeted to the lung. In some embodiments, administration is pulmonary.

Hyperactivation of melanotrophs in the pituitary gland and increased levels of plasma α-MSH have been detected in an animal model of chronic stress. Chronic fatigue syndrome (CFS) is associated with chronic stress and CFS patients with a disease duration of less than 5 years have significantly higher levels of α-MSH in their peripheral blood than healthy controls. Shishioh-Ikejima et al BMC Neurology 10:73 (2010).

### Insulin.

Insulin is produced in the pancreas by B-cells of the islets of Langerhans and released upon increase of the blood glucose level. Insulin signaling modulates cellular glucose uptake and storage (as glycogen) in the liver, muscle, and fat tissue by regulating membrane glucose transporters. In β-cells proinsulin is synthesized as a single chain, a 110 amino acid proprecursor that contains a 24 amino acid signal sequence and an 86 amino acid proinsulin. Insulin is synthesized from the proinsulin precursor molecule by the action of proteolytic enzymes, prohormone convertases PC1 and PC2 and exoprotease carboxypeptidase E. Following removal of the signal peptide, the proinsulin peptide undergoes further proteolysis to generate mature insulin, a 51 amino acid (molecular weight: 5,808 Da) disulfide-linked dimer that consists of a 30 amino acid B chain (amino acid 25-54) bound to a 21 amino acid A chain (amino acid 90-110). The 34 amino acid intervening peptide (amino acid 55-89) that connects the B and A chains and allows for the correct formation of the intra-chain disulphide bonds is referred to as the C-peptide. Human proinsulin shares 84% and 80% amino acid sequence identity with rat and bovine proinsulin, respectively. Most of the sequence variation between species occurs in the region of the C-peptide.

Low insulin levels or lack of insulin are associated with type 2 and type 1 diabetes mellitus, respectively. These conditions are associated with an increased risk for microvascular complications such as retinopathy, nephropathy, and peripheral neuropathy. Patients with type 1 diabetes depend on external insulin (commonly injected subcutaneously) because they lack the insulin hormone. Patients with type 2 diabetes are often insulin resistant. Over 40% of patients with Type 2 diabetes require insulin as part of their diabetes management plan. In type 2 diabetes, an elevated proinsulin-to-insulin ratio in blood circulation has been detected, likely as a consequence of compromised proteolytic processing of insulin precursor. Proinsulin stimulates amylin secretion by β-cells and amyloid formation in pancreatic islets. Insulin has been shown to be produced inside the brain and reduced levels of these proteins are linked to Alzheimer's disease. De la Monte S M et al. J. Alzheimers Dis. 7:45-61 (2005), Steen E et al. J. Alzheimers Dis. 7:63-80 (2005). Insulin in the brain enhances learning and memory. Benedict C et al. Psychoneuroendocrinology 29:1326-34 (2004).

Neonatal diabetes (ND), developing within the first few weeks or months of life, is a very rare condition with an incidence of 1 in 300,000 to 500,000 live births. ND can be a potentially devastating metabolic disorder characterized by mild to severe hyperglycemia with low levels of circulating insulin. Disease presentation can be transient, transient neonatal diabetes (TND) or permanent, permanent neonatal diabetes (PND). Over 50% of TND cases are associated with abnormalities of an imprinted region on chromosome 6q24, whereas mutations in the two subunits Kir6.2 and SUR 1) of the ATP-sensitive K⁺ channel of pancreatic β-cell s are a common cause of both TND and PND. Few cases of PND are associated with mutations in the genes encoding glucokinase (GCK) and insulin promoter factor-1 (IPF-1), and rare mutations in PTF1A, FOXP3, GLIS3, TCF2, and EIF2AK3. Spontaneous mutations are common in ND, with 80% of the mutations in KCNJ11 (encoding Kir6.2) occurring de novo. Missense mutations in insulin and its precursors, preproinsulin and proinsulin, affecting insulin structure and biosynthesis are also a cause of ND. Støy J et al. PNAS 104:15040-15044 (2007), Polak M et al. Diabetes 57:1115-1119 (2008), Colombo C et al. J Clin Invest. 118:2148-2156 (2008), Nishi, M et al J Diabetes Invest 2:92-100. (2011). Mutations are either inherited in an autosomal dominant manner or occur de novo. The mutations are in critical regions of the preproinsulin molecule and are predicted to prevent normal folding and progression of proinsulin in the insulin secretory pathway. The abnormally folded proinsulin molecule may induce the unfolded protein response and undergo degradation in the endoplasmic reticulum, leading to severe endoplasmic reticulum stress and potentially β cell death by apoptosis. Akita and Munich mouse models that have dominant-acting missense mutations in the Ins2 gene show loss of β cell function and mass and one of the human missense mutations is identical to that in the Akita mouse model. The human missense mutations generate polypeptides that display unpaired reactive cysteine residues and are very likely to act in a dominant manner analogous to the Akita and/or Munich mouse Ins2 mutations to disrupt insulin biosynthesis and induce endoplasmic reticulum (ER) stress.

Provided herein, are methods of treating a subject having neonatal diabetes, the methods comprising administering to a subject in need of such treatment a composition comprising a modified nucleic acid encoding an insulin precursor or a partially or fully processed form thereof in an amount sufficient to treat neonatal diabetes.

Further provided herein, are methods of preventing the development of neonatal diabetes, or delaying the onset of neonatal diabetes in a subject suspected of developing neonatal diabetes, the methods comprising administering to a subject in need of such treatment a composition comprising a modified nucleic acid encoding an insulin precursor or a partially or fully processed form thereof in an amount sufficient to prevent the development of neonatal diabetes, or to delay the onset of neonatal diabetes. In certain embodiments, the subject is an infant. In some embodiments, the infant is less than one month old, less than two months old, less than three months old, less than four months old, less than five months old, or less than one year old.

In certain embodiments, the subject suspected of developing early onset obesity was genetically tested for the presence of one or more mutations in the insulin gene. In certain embodiments, testing comprises DNA sequencing of one or more regions of the subject's insulin gene.

### Granulocyte Colony-Stimulating Factor (G-CSF).

Granulocyte colony-stimulating factor (G-CSF, GCSF, CSF 3) is a cytokine growth factor produced primarily by monocytes and macrophages (and neutrophils, fibroblasts, endothelial cells, and bone marrow stromal cells) upon activation by endotoxin, TNF-α, IFN-γ (and IL-1, IL-17 and GM-CSF) to stimulate the bone marrow (hematopoietic progenitors of neutrophil/granulocyte lineage) to produce granulocytes and stem cells for release into the blood. G-CSF also stimulates the survival, proliferation, differentiation, and function of neutrophil precursors and mature neutrophils via Janus kinase (JAK)/signal transducer and activator of transcription (STAT), Ras/mitogen-activated protein kinase (MAPK) and phosphatidylinositol 3-kinase (PI3K)/protein kinase B (Akt) signal transduction pathways. G-CSF can target endothelial cells to stimulate proliferation and migration. G-CSF has two forms, a 174- and 180-amino-acid-long protein. The 174-amino acid polypeptide is more abundant in vivo and shows higher biological activity. In unstimulated cells G-CSF mRNA is unstable and it is thought that the presence of a G-CSF factor stem-loop destabilizing element enhances the rate of shortening of the poly(A) tail. Putland R A et al. Mol Cell Biol 22:1664-1673 (2002).

The amino acid sequence of G-CSF (long form including signal peptide) is set forth in (SEQ ID No: 13):

(SEQ ID NO: 13)
MAGPATQSPMKLMALQLLLWHSALWTVQEATPLGPASSLPQSFLLKCLEQV
RKIQGDGAALQEKLVSECATYKLCHPEELVLLGHSLGIPWAPLSSCPSQALQLAGCLSQL
HSGLFLYQGLLQALEGISPELGPTLDTLQLDVADFATTIWQQMEELGMAPALQPTQGAM
PAFASAFORRAGGVLVASHLQSFLEVSYRVLRHLAQP

The 174-amino acid form of G-CSF has been used in recombinant from for the development of pharmaceutical products. Recombinant human G-CSF is synthesized in E. coli. Filgrastim (Neupogen®, Nivestim®, Ratiograstim®, Zarzio®) and PEG-filgrastim (a pegylated form, Neulasta®) are two commercially-available forms of rhG-CSF (recombinant human G-CSF). Filgrastim is a non-glycosylated 175 amino acid polypeptide (identical to human G-CSF except for the addition of an N-terminal methionine that is necessary for expression in E. coli), and has a molecular weight of 18,800 Da. G-CSF isolated from a human cell is glycosylated.

The amino acid sequence of G-CSF (175 amino acid form, Filgrastim) is set forth in (SEQ ID No: 14):

(SEQ ID NO: 14)
MTPLGPASSLPQSFLLKCLEQVRKIQGDGAALQEKLCATYKLCHPEELVLLG
HSLGIPWAPLSSCPSQALQLAGCLSQLHSGLFLYQGLLQALEGISPELGPTLDTLQLDVA
DFATTIWQQMEELGMAPALQPTQGAMPAFASAFQRRAGGVLVASHLQSFLEVSYRVLR
HLAQP

Another form of recombinant human G-CSF, called lenograstim (Granocyte®), is synthesized in Chinese Hamster Ovary (CHO) cells. As this is a mammalian cell expression system, lenograstim is indistinguishable from the 174-amino acid natural human G-CSF. Recombinant G-CSF has been used in humans for over ten years and is generally well tolerated.

G-CSF stimulates the production of white blood cells (WBC). The G-CSF-receptor (CD114, G-CSFR) is present on precursor cells in the bone marrow (of neutrophilic granulocyte lineage) and initiates proliferation and differentiation into mature granulocytes in response to stimulation by G-CSF. In addition, G-CSF can enhance the survival and activate the immunological functions of mature neutrophils. G-CSF is also a potent inducer of hematopoietic stem cells (HSCs) mobilization from the bone marrow into the bloodstream. Recombinant G-CSF is used to increase the number of hematopoietic stem cells (HSC) in the blood of a donor before collection by leukapheresis for use in hematopoietic stem cell transplantation. It may also be given to the recipient, to compensate for conditioning regimens. Recipients may be given chemotherapy or irradiation to aid the eradication of the patient's disease prior to the infusion of HSC and/or to suppress immune reactions. Recombinant forms of G-CSF are used in cancer patients to accelerate recovery from neutropenia (low white blood cell count) after chemotherapy, allowing higher-intensity treatment regimens. Chemotherapy can cause myelosuppression and unacceptably low levels of white blood cells, making patients prone to infections and sepsis. Complications associated with a low white blood cell count are the most common causes of dose reductions or delays in chemotherapy. Link et al. Cancer 92:1354-1367 (2001), Lyman et al. J Clin Oncol. 21:4524-4531 (2003), Lyman et al. Am J Med. 112:406-411 (2002). Filgrastim (Neupogen®) was approved in 1991 by the U.S. FDA for the treatment of patients suffering from chemotherapy-induced neutropenia. In 1993, lenograstim (Granocyte®) was approved in Europe.

Provided herein, are methods of increasing the number of hematopoietic stem cells (HSC) in the blood, the method comprising administering to a HSC donor subject a composition comprising a modified nucleic acid encoding a G-CSF precursor or a partially or fully processed form thereof in an amount sufficient to increasing the number of HSCs. In certain embodiments, the HSCs derived from the donor subject are used for allogenic HSC transplantation into a recipient subject. In certain embodiments, the blood of the donor subject is collected by leukapheresis. In some embodiments, an increase in the number of HSCs is determined by analysis of the blood of the donor subject.

Provided herein, are methods of to accelerate recovery from neutropenia, the method comprising administering to a subject in need of such recovery a composition comprising a modified nucleic acid encoding a G-CSF precursor or a partially or fully processed form thereof in an amount sufficient to accelerate recovery from neutropenia. In certain embodiments, the subject in need of recovery is a cancer patient. In certain embodiments, the cancer patient has undergone a conditioning regimen. In some embodiments, the conditioning regiment comprises chemotherapy or irradiation.

Provided herein, are methods to prevent infection and/or sepsis in a subject at risk of developing infection and/or sepsis, the method comprising administering to a subject in need of such prevention a composition comprising a modified nucleic acid encoding a G-CSF precursor or a partially or fully processed form thereof in an amount sufficient to prevent infection and/or sepsis. In certain embodiments, the subject at risk of developing infection and/or sepsis is a cancer patient. In certain embodiments, the cancer patient has undergone a conditioning regimen. In some embodiments, the conditioning regiment comprises chemotherapy or irradiation.

Further provided herein, are methods to treat infection and/or sepsis in a subject, the method comprising administering to a subject in need of such treatment a composition comprising a modified nucleic acid encoding a G-CSF precursor or a partially or fully processed form thereof in an amount sufficient to treat an infection and/or sepsis. In certain embodiments, the subject in need of treatment is a cancer patient. In certain embodiments, the cancer patient has undergone a conditioning regimen. In some embodiments, the conditioning regiment comprises chemotherapy or irradiation.

Further provided herein, are methods to prevent a dose reduction and/or delay in chemotherapy and/or irradiation therapy during the treatment of a cancer patient, the method comprising administering to a subject in need of such prevention a composition comprising a modified nucleic acid encoding a G-CSF precursor or a partially or fully processed form thereof in an amount sufficient to prevent a dose reduction and/or delay in chemotherapy and/or irradiation therapy during the treatment. In certain embodiments, the cancer patient exhibits neutropenia. In certain embodiments, the cancer patient is at risk to develop an infection and/or sepsis.

Neutropenia is also considered a major factor in delayed wound healing. Wound healing proceeds through three phases: an inflammatory phase, a proliferative phase, and a remodeling phase. During the inflammatory phase a moist environment is generated in which granulocytes cleanse and free the wound from cellular debris, foreign bodies, and bacteria, aided by macrophages that stimulate endothelial cell and fibroblast migration. The migration event initiates angiogenesis and fibroplasias. During the proliferative phase, fibroblasts appear that facilitate wound contraction and restructuring of the extracellular matrix. The wound is supplied with nutrients and oxygen and catabolites are removed. The remodeling phase begins 20-30 days after injury and may continue for 12-24 months thereafter. It involves collagen degradation and synthesis of new repair matrix components. Fibroblasts transform into myofibroblasts that provide the contractile capacity necessary to reduce scarring. If a wound does not heal within 2-4 weeks it can become chronic. Delayed wound healing represents a major clinical problem for patients receiving radiotherapy, chemotherapy, steroids, and/or ultraviolet radiation. Further, patients with microvascular disorders, such as diabetes, can suffer from delayed wound healing. In these patients, neutropenia and neutrophil dysfunction can lead to severe or chronic bacterial infections. Poor wound healing remains a difficult clinical problem.

G-CSF has been proved to be efficient in treating neutropenia in a number of clinical settings. G-CSF stimulates the production of polymorphonuclear neutrophils (PMN) and also enhances PMN function. G-CSF may enhance PMN function by a number of different ways, e.g. by (i) enhancing CD11b and FcγRi expression; (ii) enhancing PMN adhesion; (iii) phagocytosis; (iv) degranulation; and (v) chemokinesis. G-CSF may also modulate peripheral mature PMN activation and/or regulate PMN chemokine responsiveness. Anderlini P et al. Blood 88:2819-2825 (1996), Betsuyacu T et al. J Clin Invest 103:825-832 (1999), Hakanson L et al. Br J Haematol 98:603-611 (1997), Gericke G H et al. J Leukocyte Biol 57:455-61 (1995), Hoglund M et al. Eur J Haematol 58:195-202 (1997), Xu S et al. Br J Haematol 93:558-68 (1996), Yong K L Br J Haematol 94:40-7 (1996), Metcalf D et al. Blood 88:3755-3764 (1996). Favorable effects of subcutaneous application of recombinant G-CSF have been reported, including reduction of bacterial translocation due to burn wound sepsis and acceleration of wound healing in neutropenic patient using recombinant human G-CSF (hG-CSF). Yalcin O et al. Surg Today 27:154-158 (1997), Harada T et al Burns 24:120-122 (1998), Cody D T et al. Head Neck 21:172-175 (1999). G-CSF has also been shown to improve incisional wound healing in animal models of third degree burns. Eroglu E et al. Tohoku J Exp Med 204:11-16 (2004). Plasmid DNA encoding hG-CSF gene was successfully delivered into surgical wound sites, resulting in G-CSF expression. Both local and systemic gene delivery with either plasmid DNA alone (naked DNA) or with cationic liposome DNA complexes specifically to sites of wounded skin was performed. Meuli M et al. J Invest Dermatology 116, 131-135 (2001).

Provided herein, are methods to accelerate wound healing, the method comprising administering to a subject exhibiting delayed wound healing a composition comprising a modified nucleic acid encoding a G-CSF precursor or a partially or fully processed form thereof in an amount sufficient to accelerate wound healing. In certain embodiments, the subject exhibits neutropenia. In certain embodiments, the subject exhibits neutrophil dysfunction. In certain embodiments, the subject exhibits severe or chronic bacterial infections. In certain embodiments, the subject has received or is receiving a therapy. In certain embodiments, the therapy is radiotherapy, chemotherapy, steroids, or ultraviolet radiation. In certain embodiments, the patient suffers from a microvascular disorder. In some embodiments, the microvascular disorder is diabetes. In some embodiments, the wound is an ulcer. In a specific embodiment, the wound is a diabetic foot ulcer. In certain embodiments, the subject has one or more burn wounds. In certain embodiments, the administration is local or systemic. In certain embodiments, the administration is subcutaneous. In certain embodiments, the administration is topical.

G-CSF can also act on neuronal cells as a neurotrophic factor in the central nervous system (CNS). The G-CSF-receptor is expressed by neurons in the brain and spinal cord. G-CSF has been shown to decrease infectious episodes and acute inflammatory response in patients with a variety of brain pathologies, including brain tumors, acute traumatic brain injury, and cerebral hemorrhage. Shyu W C Circulation 110:1847-1854 (2004), Schneider A et al. J Clin Invest 115:2083-2098 (2005), Kawada H et al. Circulation 113:701-710 (2006). Recombinant G-CSF is currently under investigation for cerebral ischemia (stroke) in a clinical phase IIb (AXIS 2: AX200 for the Treatment of Ischemic Stroke, NCT00927836 Sygnis Bioscience, Germany). As a neuroprotectant G-CSF is considered to be more effective in the early phase of cerebral ischemia and during reperfusion. G-CSF was found to be neuroprotective in experimental models of thromboembolic stroke (thromboembolic occlusion (TE) of the middle cerebral artery in male Wistar rats) when given early after TE. Kollmar R Exp & Transl Stroke Med 2:9 (2010). G-CSF's neuroprotective and regenerative properties are likely a result of a combination of anti-apoptotic activity on neurons, stimulation of neurogenesis, enhancement of vessel formation, mobilization of bone marrow derived cells and systemic anti-inflammatory effects. G-CSF receptor and G-CSF itself are expressed in alpha motoneurons. G-CSF has been shown to protect motoneurons from apoptosis, and to improve outcome in a SOD1 (G93A) transgenic mouse model for the motorneuron disease amyotrophic lateral sclerosis (ALS, Lou Gehrig's disease), which affects 5 out of 100,000 people worldwide. Henriques et al. BMC Neuroscience 11:25 (2010).

G-CSF has also been proposed as a candidate for the treatment of Alzheimer's disease (AD). Tsai K J et al. J Exp Med. 204:1273-80 (2007). AD affects more than 12 million patients worldwide. AD brains develop neurofibrillary tangles and senile plaques. β-Amyloid (Aβ) is the major extracellular component of the senile plaques. Mattson, MP Nature 430:631-639 (2004), Blennow K et al. Lancet 368:387-403 (2006). Clinical treatments for AD are largely symptomatic and none are capable of stopping the progression of AD. Acetylcholinesterase inhibitors improve cognitive ability and psychotropic drugs modify patient behaviors. Treatments that focus on delaying the onset of symptoms and slowing the rate of disease progression include (i) tacrine (Cognex®), the first FDA-approved drug for AD therapy; (ii) memantine (Namenda®), an N-methyl-D-aspartate antagonist; and (iii) antioxidants such as vitamin E. Other AD therapies include anti-amyloid immunotherapy, amyloid vaccination, and the use of secretase inhibitors that prevent the formation of Aβ and neurofibrillary tangles. However, these therapies all cause side effects and clinical problems. Francis P T et al. Trends Pharmacol Sci. 26:104-111 (2005), Schenk D Nat. Rev. Neurosci. 3:824-828 (2002), Citron M Nat. Rev. Neurosci. 5:677-685 (2004). Animal studies employing Aβ aggregate-induced AD mouse models suggest that G-CSF treatment has potential applications in AD. G-CSF induced stem cell release from the bone marrow, stimulated neurogenesis surrounding the Aβ plaques in mouse brains, and improved the neurological function of AD mice, judged by the enhanced levels of acetylcholine in the brains of Tg2576 mice. Tsai K J et al. J Exp Med. 204:1273-80 (2007).

Provided herein, are methods of treating a subject exhibiting acute brain pathology, the methods comprising administering to a subject in need of such treatment a composition comprising a modified nucleic acid encoding a G-CSF precursor or a partially or fully processed form thereof in an amount sufficient to treat the acute brain pathology. In certain embodiments, the acute brain pathology is acute traumatic brain injury, cerebral hemorrhage or cerebral ischemia (stroke). In certain embodiments, the modified nucleic acid encoding a G-CSF precursor or a partially or fully processed form thereof is administered immediately after or shortly after the subject exhibits an acute brain pathology. In certain embodiments, the modified nucleic acid encoding a G-CSF precursor or a partially or fully processed form thereof is administered directly into the brain. In other embodiments, administration is systemic.

Provided herein, are methods of treating a subject having a neurodegenerative disease, the methods comprising administering to a subject in need of such treatment a composition comprising a modified nucleic acid encoding a G-CSF precursor or a partially or fully processed form thereof in an amount sufficient to treat the neurodegenerative disease.

Further provided herein, are methods of preventing the development of a neurodegenerative disease, or delaying the onset of a neurodegenerative disease in a subject suspected of developing neurodegenerative disease, the methods comprising administering to a subject in need of such prevention a composition comprising a modified nucleic acid encoding a G-CSF precursor or a partially or fully processed form thereof in an amount sufficient to prevent the development of the neurodegenerative disease, or to delay the onset of the neurodegenerative disease. In certain embodiments, the subject is an adult over 40, 50, 60, or 70 year of age. In certain embodiments, the neurodegenerative disease is a motoneuron disease. In specific embodiments, the motoneuron disease is amyotrophic lateral sclerosis (ALS, Lou Gehrig's disease). In other embodiments, the disease is Alzheimer's disease (AD). In certain embodiments, the modified nucleic acid encoding a G-CSF precursor or a partially or fully processed form thereof is administered directly into the brain. In other embodiments, administration is systemic.

G-CSF has been shown to improve cardiac function after myocardial infarction (MI) by mobilizing bone marrow cells and/or by protecting cardiomyocytes from apoptotic cell death. G-CSF has also been tested for its role in collateral artery growth (arteriogenesis) in a murine MI model. G-CSF administration after MI stimulates arteriogenesis, attenuates ischemic cardiomyopathy and results in a significant improvement of post-MI survival. Deindl E et al. FASEB J 20:956-958 (2006).

Provided herein, are methods of treating a subject having myocardial infarction (MI), the methods comprising administering to a subject in need of such treatment a composition comprising a modified nucleic acid encoding a G-CSF precursor or a partially or fully processed form thereof in an amount sufficient to treat MI.

Provided herein, are methods of improving cardiac function in a subject having myocardial infarction (MI), the methods comprising administering to a subject in need of such improvement a composition comprising a modified nucleic acid encoding a G-CSF precursor or a partially or fully processed form thereof in an amount sufficient to improve cardiac function.

Provided herein, are methods of protecting cardiomyocytes from apoptotic cell death in a subject having myocardial infarction (MI), the methods comprising administering to a subject in need of such improvement a composition comprising a modified nucleic acid encoding a G-CSF precursor or a partially or fully processed form thereof in an amount sufficient to protect cardiomyocytes from apoptotic cell death.

Provided herein, are methods of promoting collateral artery growth (arteriogenesis) in a subject having myocardial infarction (MI), the methods comprising administering to a subject in need of such improvement a composition comprising a modified nucleic acid encoding a G-CSF precursor or a partially or fully processed form thereof in an amount sufficient to promote collateral artery growth (arteriogenesis). In certain embodiments, the modified nucleic acid encoding a G-CSF precursor or a partially or fully processed form thereof is administered directly into myocardium. In other embodiments, administration is systemic.

G-CSF also exerts anti-inflammatory and pro-Th2 effects. G-CSF, when administered at the onset of clinical signs, provided durable protection from experimental autoimmune encephalomyelitis (EAE), a murine model (SJL/J mice) for multiple sclerosis that is driven by Th1-oriented auto-aggressive cells. G-CSF reduces the T cell infiltration and autoimmune inflammation within the CNS. G-CSF-treated mice displayed limited demyelination, reduced recruitment of T cells to the CNS, very discrete autoimmune inflammation, and low levels of CNS mRNA levels of cytokines and chemokines. G-CSF also limited the production of TNF-α, a cytokine associated with early CNS infiltration and neurological deficit. Zavala F et al. J Immunol 168:2011-2019 (2002). Treatment with G-CSF has also been shown to protect mice from the development of spontaneous systemic lupus, another autoimmune disease. Zavala, F et al. J Immunol 163:5125 (1999).

Provided herein, are methods of treating a subject having an autoimmune disease, the methods comprising administering to a subject in need of such treatment a composition comprising a modified nucleic acid encoding a G-CSF precursor or a partially or fully processed form thereof in an amount sufficient to treat the autoimmune disease. In certain embodiments, the autoimmune disease is multiple sclerosis (MS) or systemic lupus.

### Modified Nucleic Acids.

This invention provides nucleic acids, including RNAs such as mRNAs that contain one or more modified nucleosides (termed “modified nucleic acids”), which have useful properties including the lack of a substantial induction of the innate immune response of a cell into which the mRNA is introduced. Because these modified nucleic acids enhance the efficiency of protein production, intracellular retention of nucleic acids, and viability of contacted cells, as well as possess reduced immunogenicity, these nucleic acids having these properties are termed “enhanced nucleic acids” herein.

The term “nucleic acid,” in its broadest sense, includes any compound and/or substance that is or can be incorporated into an oligonucleotide chain. Exemplary nucleic acids for use in accordance with the present invention include, but are not limited to, one or more of DNA, RNA, hybrids thereof, RNAi-inducing agents, RNAi agents, siRNAs, shRNAs, miRNAs, antisense RNAs, ribozymes, catalytic DNA, RNAs that induce triple helix formation, aptamers, vectors, etc., described in detail herein.

Provided are modified nucleic acids containing a translatable region and one, two, or more than two different nucleoside modifications. In some embodiments, the modified nucleic acid exhibits reduced degradation in a cell into which the nucleic acid is introduced, relative to a corresponding unmodified nucleic acid. Exemplary nucleic acids include ribonucleic acids (RNAs), deoxyribonucleic acids (DNAs), threose nucleic acids (TNAs), glycol nucleic acids (GNAs), peptide nucleic acids (PNAs), locked nucleic acids (LNAs) or a hybrid thereof. In preferred embodiments, the modified nucleic acid includes messenger RNAs (mRNAs). As described herein, the nucleic acids of the invention do not substantially induce an innate immune response of a cell into which the mRNA is introduced.

In some embodiments, modified nucleosides include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine.

In some embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In other embodiments, modified nucleosides include 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In certain embodiments it is desirable to intracellularly degrade a modified nucleic acid introduced into the cell, for example if precise timing of protein production is desired. Thus, the invention provides a modified nucleic acid containing a degradation domain, which is capable of being acted on in a directed manner within a cell.

In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

Other components of nucleic acid are optional, and are beneficial in some embodiments. For example, a 5′ untranslated region (UTR) and/or a 3′UTR are provided, wherein either or both may independently contain one or more different nucleoside modifications. In such embodiments, nucleoside modifications may also be present in the translatable region. Also provided are nucleic acids containing a Kozak sequence.

Additionally, provided are nucleic acids containing one or more intronic nucleotide sequences capable of being excised from the nucleic acid.

Further, provided are nucleic acids containing an internal ribosome entry site (IRES). An IRES may act as the sole ribosome binding site, or may serve as one of multiple ribosome binding sites of an mRNA. An mRNA containing more than one functional ribosome binding site may encode several peptides or polypeptides that are translated independently by the ribosomes (“multicistronic mRNA”). When nucleic acids are provided with an IRES, further optionally provided is a second translatable region. Examples of IRES sequences that can be used according to the invention include without limitation, those from picornaviruses (e.g. FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

### Prevention or Reduction of Innate Cellular Immune Response Activation Using Modified Nucleic Acids.

The term “innate immune response” includes a cellular response to exogenous single stranded nucleic acids, generally of viral or bacterial origin, which involves the induction of cytokine expression and release, particularly the interferons, and cell death. Protein synthesis is also reduced during the innate cellular immune response. While it is advantageous to eliminate the innate immune response in a cell, the invention provides modified mRNAs that substantially reduce the immune response, including interferon signaling, without entirely eliminating such a response. In some embodiments, the immune response is reduced by 10%, 20%, 30%, 40%, 50%, 60%, 70%, 80%, 90%, 95%, 99%, 99.9%, or greater than 99.9% as compared to the immune response induced by a corresponding unmodified nucleic acid. Such a reduction can be measured by expression or activity level of Type 1 interferons or the expression of interferon-regulated genes such as the toll-like receptors (e.g., TLR7 and TLR8). Reduction of innate immune response can also be measured by decreased cell death following one or more administrations of modified RNAs to a cell population; e.g., cell death is 10%, 25%, 50%, 75%, 85%, 90%, 95%, or over 95% less than the cell death frequency observed with a corresponding unmodified nucleic acid. Moreover, cell death may affect fewer than 50%, 40%, 30%, 20%, 10%, 5%, 1%, 0.1%, 0.01% or fewer than 0.01% of cells contacted with the modified nucleic acids.

The invention provides for the repeated introduction (e.g., transfection) of modified nucleic acids into a target cell population, e.g., in vitro, ex vivo, or in vivo. The step of contacting the cell population may be repeated one or more times (such as two, three, four, five or more than five times). In some embodiments, the step of contacting the cell population with the modified nucleic acids is repeated a number of times sufficient such that a predetermined efficiency of protein translation in the cell population is achieved. Given the reduced cytotoxicity of the target cell population provided by the nucleic acid modifications, such repeated transfections are achievable in a diverse array of cell types.

### Polypeptide Variants.

Provided are nucleic acids that encode variant polypeptides, which have a certain identity with a reference polypeptide sequence. The term “identity” as known in the art, refers to a relationship between the sequences of two or more peptides, as determined by comparing the sequences. In the art, “identity” also means the degree of sequence relatedness between peptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

In some embodiments, the polypeptide variant has the same or a similar activity as the reference polypeptide. Alternatively, the variant has an altered activity (e.g., increased or decreased) relative to a reference polypeptide. Generally, variants of a particular polynucleotide or polypeptide of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular reference polynucleotide or polypeptide as determined by sequence alignment programs and parameters described herein and known to those skilled in the art.

As recognized by those skilled in the art, protein fragments, functional protein domains, and homologous proteins are also considered to be within the scope of this invention. For example, provided herein is any protein fragment of a reference protein (meaning a polypeptide sequence at least one amino acid residue shorter than a reference polypeptide sequence but otherwise identical) 5, 10, 15, 20, 25, 30, 35, 40, 45, 50, 55, 60, 70, 80, 90, 100 or greater than 100 amino acids in length. In another example, any protein that includes a stretch of about 20, about 30, about 40, about 50, or about 100 amino acids which are about 40%, about 50%, about 60%, about 70%, about 80%, about 90%, about 95%, about 98%, or about 100% identical to any of the sequences described herein can be utilized in accordance with the invention. In certain embodiments, a protein sequence to be utilized in accordance with the invention includes 2, 3, 4, 5, 6, 7, 8, 9, 10, or more mutations as shown in any of the sequences provided or referenced herein.

### Polypeptide Libraries.

Also provided are polynucleotide libraries containing nucleoside modifications, wherein the polynucleotides individually contain a first nucleic acid sequence encoding a polypeptide, such as an antibody, protein binding partner, scaffold protein, and other polypeptides known in the art. Preferably, the polynucleotides are mRNA in a form suitable for direct introduction into a target cell host, which in turn synthesizes the encoded polypeptide.

In certain embodiments, multiple variants of a protein, each with different amino acid modification(s), are produced and tested to determine the best variant in terms of pharmacokinetics, stability, biocompatibility, and/or biological activity, or a biophysical property such as expression level. Such a library may contain 10, 10², 10³, 10⁴, 10⁵, 10⁶, 10⁷, 10⁸, 10⁹, or over 10⁹ possible variants (including substitutions, deletions of one or more residues, and insertion of one or more residues).

### Polypeptide-Nucleic Acid Complexes.

Proper protein translation involves the physical aggregation of a number of polypeptides and nucleic acids associated with the mRNA. Provided by the invention are complexes containing conjugates of protein and nucleic acids, containing a translatable mRNA having one or more nucleoside modifications (e.g., at least two different nucleoside modifications) and one or more polypeptides bound to the mRNA. Generally, the proteins are provided in an amount effective to prevent or reduce an innate immune response of a cell into which the complex is introduced.

### Targeting Moieties.

In embodiments of the invention, modified nucleic acids are provided to express a protein-binding partner or a receptor on the surface of the cell, which functions to target the cell to a specific tissue space or to interact with a specific moiety, either in vivo or in vitro. Suitable protein-binding partners include antibodies and functional fragments thereof, scaffold proteins, or peptides. Additionally, modified nucleic acids can be employed to direct the synthesis and extracellular localization of lipids, carbohydrates, or other biological moieties.

### Untranslatable Modified Nucleic Acids; Vaccines.

As described herein, provided are mRNAs having sequences that are substantially not translatable. Such mRNA is effective as a vaccine when administered to a mammalian subject.

Also provided are modified nucleic acids that contain one or more noncoding regions. Such modified nucleic acids are generally not translated, but are capable of binding to and sequestering one or more translational machinery component such as a ribosomal protein or a transfer RNA (tRNA), thereby effectively reducing protein expression in the cell. The modified nucleic acid may contain a small nucleolar RNA (sno-RNA), micro RNA (miRNA), small interfering RNA (siRNA) or Piwi-interacting RNA (piRNA).

Additionally, certain modified nucleosides, or combinations thereof, when introduced into modified nucleic acids activate the innate immune response. Such activating modified nucleic acids, e.g., modified RNAs, are useful as adjuvants when combined with polypeptide or other vaccines. In certain embodiments, the activated modified mRNAs contain a translatable region which encodes for a polypeptide sequence useful as a vaccine, thus providing the ability to be a self-adjuvant.

### Modified Nucleic Acid Synthesis.

Nucleic acids for use in accordance with the invention may be prepared according to any available technique including, but not limited to chemical synthesis, enzymatic synthesis, which is generally termed in vitro transcription, enzymatic or chemical cleavage of a longer precursor, etc. Methods of synthesizing RNAs are known in the art (see, e.g., Gait, M. J. (ed.) Oligonucleotide synthesis: a practical approach, Oxford (Oxfordshire), Washington, DC: IRL Press, 1984; and Herdewijn, P. (ed.) Oligonucleotide synthesis: methods and applications, Methods in Molecular Biology, v. 288 (Clifton, N.J.) Totowa, N.J.: Humana Press, 2005; both of which are incorporated herein by reference).

Modified nucleic acids need not be uniformly modified along the entire length of the molecule. Different nucleotide modifications and/or backbone structures may exist at various positions in the nucleic acid. One of ordinary skill in the art will appreciate that the nucleotide analogs or other modification(s) may be located at any position(s) of a nucleic acid such that the function of the nucleic acid is not substantially decreased. A modification may also be a 5′ or 3′ terminal modification. The nucleic acids may contain at a minimum one and at maximum 100% modified nucleotides, or any intervening percentage, such as at least 50% modified nucleotides, at least 80% modified nucleotides, or at least 90% modified nucleotides.

Generally, the length of a modified mRNA of the present invention is greater than 30 nucleotides in length. In another embodiment, the RNA molecule is greater than 35, 40, 45, 50, 60, 75, 100, 125, 150, 175, 200, 250, 300, 350, 400, 450, 500, 600, 700, 800, 900, 1000, 1100, 1200, 1300, 1400, 1500, 1800, 2000, 3000, 4000, or 5000 nucleotides, or greater than 5000 nucleotides.

### Uses of Modified Nucleic Acids.

### Therapeutic Agents

Provided are compositions, methods, kits, and reagents for treatment or prevention of disease or conditions in humans and other mammals. The active therapeutic agents of the invention include modified nucleic acids, cells containing modified nucleic acids or polypeptides translated from the modified nucleic acids, polypeptides translated from modified nucleic acids, and cells contacted with cells containing modified nucleic acids or polypeptides translated from the modified nucleic acids.

Provided are methods of inducing translation of a recombinant polypeptide in a cell population using the modified nucleic acids described herein. Such translation can be in vivo, ex vivo, in culture, or in vitro. The cell population is contacted with an effective amount of a composition containing a nucleic acid that has at least one nucleoside modification, and a translatable region encoding the recombinant polypeptide. The population is contacted under conditions such that the nucleic acid is localized into one or more cells of the cell population and the recombinant polypeptide is translated in the cell from the nucleic acid.

An effective amount of the composition is provided based, at least in part, on the target tissue, target cell type, means of administration, physical characteristics of the nucleic acid (e.g., size, and extent of modified nucleosides), and other determinants. In general, an effective amount of the composition provides efficient protein production in the cell, preferably more efficient than a composition containing a corresponding unmodified nucleic acid. Increased efficiency may be demonstrated by increased cell transfection (i.e., the percentage of cells transfected with the nucleic acid), increased protein translation from the nucleic acid, decreased nucleic acid degradation (as demonstrated, e.g., by increased duration of protein translation from a modified nucleic acid), or reduced innate immune response of the host cell.

Aspects of the invention are directed to methods of inducing in vivo translation of a recombinant polypeptide in a mammalian subject in need thereof. Therein, an effective amount of a composition containing a nucleic acid that has at least one nucleoside modification and a translatable region encoding the recombinant polypeptide is administered to the subject using the delivery methods described herein. The nucleic acid is provided in an amount and under other conditions such that the nucleic acid is localized into a cell of the subject and the recombinant polypeptide is translated in the cell from the nucleic acid. The cell in which the nucleic acid is localized, or the tissue in which the cell is present, may be targeted with one or more than one rounds of nucleic acid administration.

Other aspects of the invention relate to transplantation of cells containing modified nucleic acids to a mammalian subject. Administration of cells to mammalian subjects is known to those of ordinary skill in the art, such as local implantation (e.g., topical or subcutaneous administration), organ delivery or systemic injection (e.g., intravenous injection or inhalation), as is the formulation of cells in pharmaceutically acceptable carrier. Compositions containing modified nucleic acids are formulated for administration intramuscularly, transarterially, intraocularly, vaginally, rectally, intraperitoneally, intravenously, intranasally, subcutaneously, endoscopically, transdermally, or intrathecally. In some embodiments, the composition is formulated for extended release.

### Topical Delivery Applied to the Skin.

The skin is an ideal target site for nucleic acid delivery. It is readily accessible, and gene expression may be restricted not only to the skin, potentially avoiding nonspecific toxicity, but also to specific layers and cell types within the skin. The site of cutaneous expression of the delivered nucleic acid will depend on the route of nucleic acid delivery. Three routes are commonly considered to deliver nucleic acids to the skin: (i) topical application (e.g. for local/regional treatment); (ii) intradermal injection (e.g. for local/regional treatment); and (iii) systemic delivery (e.g. for treatment of dermatologic diseases that affect both cutaneous and extracutaneous regions). Nucleic acids can be delivered to the skin by several different approaches. Most have been shown to work for DNA, such as, topical application of non-cationic liposome-DNA complex, cationic liposome-DNA complex, particle-mediated (gene gun), puncture-mediated gene transfections, and viral delivery approaches. After gene delivery, gene products have been detected in a number of skin cell types, including but not limited to basal keratinocytes, sebaceous gland cells, dermal fibroblasts and dermal macrophages.

### Wound Management.

For wound treatment, e.g. of wounds exhibiting delayed healing, comprising administration of modified nucleic acids encoding for a G-CSF precursor or a partially or fully processed form thereof, wound management may further comprise steps carried out either prior to, concurrent with or post administration of the modified nucleic acids. For example, steps may involve cleaning and preparing the wound bed to facilitate wound healing and obtain closure of the wound. Several strategies may be used in order to promote wound healing and achieve wound closure including, but not limited to: (i) debridement, optionally repeated, sharp debridement (surgical removal of dead or infected tissue from a wound), optionally including chemical debriding agents, such as enzymes, to remove necrotic tissue; (ii) wound dressings to provide the wound with a moist, warm environment and to promote tissue repair and healing. Examples of materials that are used in formulating wound dressings include: hydrogels (e.g., Aquasorb®; Duoderm®), hydrocolloids (e.g., Aquacel®; Comfeel®), foams (e.g., LYOfoam®; Spyrosorb®), and alginates (e.g., AlgiSite®; Curasorb®); (iii) additional growth factors to stimulate cell division and proliferation and to promote wound healing e.g. becaplermin (Regranex Gel®), a human recombinant platelet-derived growth factor that is approved by the FDA for the treatment of neuropathic foot ulcers; (iv) soft-tissue wound coverage, a skin graft may be necessary to obtain coverage of clean, non-healing wounds. Examples of skin grafts that may be used for soft-tissue coverage include: autologous skin grafts, cadaveric skin graft, bioengineered skin substitutes (e.g., Apligraf®; Dermagraft®).

In certain embodiments, wound dressing formulations comprising hydrogels (e.g., Aquasorb®; Duoderm®), hydrocolloids (e.g., Aquacel®; Comfeel®), foams (e.g., LYOfoam®; Spyrosorb®), and/or alginates (e.g., AlgiSite®; Curasorb®) are provided further comprising modified nucleic acids encoding for a G-CSF precursor or a partially or fully processed form described herein.

In certain embodiments, skin grafts comprising autologous skin grafts, cadaveric skin graft, or bioengineered skin substitutes (e.g., Apligraf®; Dermagraft®) are provided further comprising modified nucleic acids encoding for a G-CSF precursor or a partially or fully processed form described herein.

The modified nucleic acids encoding for a G-CSF precursor or a partially or fully processed form described herein may be intermixed with the wound dressing formulations and/or skin grafts or may be applied separately, e.g. by soaking or spraying.

The subject to whom the therapeutic agent is administered suffers from or is at risk of developing a disease, disorder, or deleterious condition. Provided are methods of identifying, diagnosing, and classifying subjects on these bases, which may include clinical diagnosis, biomarker levels, genome-wide association studies (GWAS), and other methods known in the art.

In certain embodiments, nucleic acids encoding G-CSF are administered to subjects in need of G-CSF administration. Subjects in need of G-CSF administration may, for example, have low levels white blood cells, e.g. a neutropenic patient, G-CSF may be administered in healthy donors to boost the white blood cell count, or G-CSF may be administered to stimulate or mobilize hematopoietic stem cells ((HSC) in a subject for any reason. To assess, for example, whether a subject is in need of G-CSF administration and/or to assess the activity of administered G-CSF in vivo, whole blood of a subject may be analyzed using a complete blood count (CBC). A CBC test may comprise one or more of the following:
a. White blood cell (WBC) count: A count of the actual number of white blood cells per volume of blood.
b. White blood cell differential: Account of the types of white blood cells present in the blood: neutrophils, lymphocytes, monocytes, eosinophils, and basophils.
c. Red blood cell (RBC) count: A count of the actual number of red blood cells per volume of blood.
d. Hemoglobin level: A measure of the amount of oxygen-carrying protein in the blood.
e. Hematocrit level: A measures of the percentage of red blood cells in a given volume of whole blood.
f. Platelet count: A count of the number of platelets in a given volume of blood.
g. Mean platelet volume (MPV): A measurement of the average size of platelets. Newly produced platelets are larger and an increased MPV occurs when increased numbers of platelets are being produced in the bone marrow.
h. Mean corpuscular volume (MCV): A measurement of the average size of RBCs (e.g. RBCs are larger than normal (macrocytic) or RBCs are smaller than normal (microcytic)).
i. Mean corpuscular hemoglobin (MCH): A calculation of the average amount of oxygen-carrying hemoglobin inside a red blood cell.
j. Mean corpuscular hemoglobin concentration (MCHC): A calculation of the average concentration of hemoglobin inside a red cell (e.g. decreased MCHC values (hypochromia) or increased MCHC values (hyperchromia)).
k. Red cell distribution width (RDW): A calculation of the variation in the size of RBCs (e.g. amount of variation (anisocytosis) in RBC size and/or variation in shape (poikilocytosis) may cause an increase in the RDW).

In certain embodiments, the administered modified nucleic acid directs production of one or more recombinant polypeptides that provide a functional activity which is substantially absent in the cell in which the recombinant polypeptide is translated. For example, the missing functional activity may be enzymatic, structural, or gene regulatory in nature. In related embodiments, the administered modified nucleic acid directs production of one or more recombinant polypeptides that increases (e.g., synergistically) a functional activity which is present but substantially deficient in the cell in which the recombinant polypeptide is translated.

In other embodiments, the administered modified nucleic acid directs production of one or more recombinant polypeptides that replace a polypeptide (or multiple polypeptides) that is substantially absent in the cell in which the recombinant polypeptide is translated. Such absence may be due to genetic mutation of the encoding gene or regulatory pathway thereof. In some embodiments, the recombinant polypeptide increases the level of an endogenous protein in the cell to a desirable level; such an increase may bring the level of the endogenous protein from a subnormal level to a normal level, or from a normal level to a super-normal level.

Alternatively, the recombinant polypeptide functions to antagonize the activity of an endogenous protein present in, on the surface of, or secreted from the cell. Usually, the activity of the endogenous protein is deleterious to the subject, for example, do to mutation of the endogenous protein resulting in altered activity or localization. Additionally, the recombinant polypeptide antagonizes, directly or indirectly, the activity of a biological moiety present in, on the surface of, or secreted from the cell. Examples of antagonized biological moieties include lipids (e.g., cholesterol), a lipoprotein (e.g., low density lipoprotein), a nucleic acid, a carbohydrate, a protein toxin such as shiga and tetanus toxins, or a small molecule toxin such as botulinum, cholera, and diphtheria toxins. Additionally, the antagonized biological molecule may be an endogenous protein that exhibits an undesirable activity, such as a cytotoxic or cytostatic activity.

The recombinant proteins described herein are engineered for localization within the cell, potentially within a specific compartment such as the nucleus, or are engineered for secretion from the cell or translocation to the plasma membrane of the cell.

### Targeting Moieties.

In embodiments of the invention, modified nucleic acids are provided to express a protein-binding partner or a receptor on the surface of the cell, which functions to target the cell to a specific tissue space or to interact with a specific moiety, either in vivo or in vitro. Suitable protein-binding partners include antibodies and functional fragments thereof, scaffold proteins, or peptides. Additionally, modified nucleic acids can be employed to direct the synthesis and extracellular localization of lipids, carbohydrates, or other biological moieties.

As described herein, a useful feature of the modified nucleic acids of the invention is the capacity to reduce the innate immune response of a cell to an exogenous nucleic acid. Provided are methods for performing the titration, reduction or elimination of the immune response in a cell or a population of cells. In some embodiments, the cell is contacted with a first composition that contains a first dose of a first exogenous nucleic acid including a translatable region and at least one nucleoside modification, and the level of the innate immune response of the cell to the first exogenous nucleic acid is determined. Subsequently, the cell is contacted with a second composition, which includes a second dose of the first exogenous nucleic acid, the second dose containing a lesser amount of the first exogenous nucleic acid as compared to the first dose. Alternatively, the cell is contacted with a first dose of a second exogenous nucleic acid. The second exogenous nucleic acid may contain one or more modified nucleosides, which may be the same or different from the first exogenous nucleic acid or, alternatively, the second exogenous nucleic acid may not contain modified nucleosides. The steps of contacting the cell with the first composition and/or the second composition may be repeated one or more times. Additionally, efficiency of protein production (e.g., protein translation) in the cell is optionally determined, and the cell may be re-transfected with the first and/or second composition repeatedly until a target protein production efficiency is achieved.

### Therapeutics for Diseases and Conditions.

Provided are methods for treating or preventing a symptom of diseases characterized by missing or aberrant protein activity, by replacing the missing protein activity or overcoming the aberrant protein activity. Because of the rapid initiation of protein production following introduction of modified mRNAs, as compared to viral DNA vectors, the compounds of the present invention are particularly advantageous in treating acute diseases such as sepsis, stroke, and myocardial infarction. Moreover, the lack of transcriptional regulation of the modified mRNAs of the invention is advantageous in that accurate titration of protein production is achievable.

In some embodiments, modified mRNAs and their encoded polypeptides in accordance with the present invention may be used for therapeutic purposes. In some embodiments, modified mRNAs and their encoded polypeptides in accordance with the present invention may be used for treatment of any of a variety of diseases, disorders, and/or conditions, including but not limited to one or more of the following: autoimmune disorders (e.g. diabetes, lupus, multiple sclerosis, psoriasis, rheumatoid arthritis); inflammatory disorders (e.g. arthritis, pelvic inflammatory disease); infectious diseases (e.g. viral infections (e.g., HIV, HCV, RSV), bacterial infections, fungal infections, sepsis); neurological disorders (e.g. Alzheimer's disease, Huntington's disease; autism; Duchenne muscular dystrophy); cardiovascular disorders (e.g. atherosclerosis, hypercholesterolemia, thrombosis, clotting disorders, angiogenic disorders such as macular degeneration); proliferative disorders (e.g. cancer, benign neoplasms); respiratory disorders (e.g. chronic obstructive pulmonary disease); digestive disorders (e.g. inflammatory bowel disease, ulcers); musculoskeletal disorders (e.g. fibromyalgia, arthritis); endocrine, metabolic, and nutritional disorders (e.g. diabetes, osteoporosis); urological disorders (e.g. renal disease); psychological disorders (e.g. depression, schizophrenia); skin disorders (e.g. wounds, eczema); blood and lymphatic disorders (e.g. anemia, hemophilia); etc.

Diseases characterized by dysfunctional or aberrant protein activity include cystic fibrosis, sickle cell anemia, epidermolysis bullosa, amyotrophic lateral sclerosis, and glucose-6-phosphate dehydrogenase deficiency. The present invention provides a method for treating such conditions or diseases in a subject by introducing nucleic acid or cell-based therapeutics containing the modified nucleic acids provided herein, wherein the modified nucleic acids encode for a protein that antagonizes or otherwise overcomes the aberrant protein activity present in the cell of the subject.

Specific examples of a dysfunctional protein are the missense mutation variants of the cystic fibrosis transmembrane conductance regulator (CFTR) gene, which produce a dysfunctional protein variant of CFTR protein, which causes cystic fibrosis.

Diseases characterized by missing (or substantially diminished such that proper protein function does not occur) protein activity include cystic fibrosis, Niemann-Pick type C, B thalassemia major, Duchenne muscular dystrophy, Hurler Syndrome, Hunter Syndrome, and Hemophilia A. Such proteins may not be present, or are essentially non-functional. The present invention provides a method for treating such conditions or diseases in a subject by introducing nucleic acid or cell-based therapeutics containing the modified nucleic acids provided herein, wherein the modified nucleic acids encode for a protein that replaces the protein activity missing from the target cells of the subject. Specific examples of a dysfunctional protein are the nonsense mutation variants of the cystic fibrosis transmembrane conductance regulator (CFTR) gene, which produce a nonfunctional protein variant of CFTR protein, which causes cystic fibrosis.

Thus, provided are methods of treating cystic fibrosis in a mammalian subject by contacting a cell of the subject with a modified nucleic acid having a translatable region that encodes a functional CFTR polypeptide, under conditions such that an effective amount of the CTFR polypeptide is present in the cell. Preferred target cells are epithelial, endothelial and mesothelial cells, such as the lung, and methods of administration are determined in view of the target tissue; i.e., for lung delivery, the RNA molecules are formulated for administration by inhalation.

In another embodiment, the present invention provides a method for treating hyperlipidemia in a subject, by introducing into a cell population of the subject with a modified mRNA molecule encoding Sortilin, a protein recently characterized by genomic studies, thereby ameliorating the hyperlipidemia in a subject. The SORT1 gene encodes a trans-Golgi network (TGN) transmembrane protein called Sortilin. Genetic studies have shown that one of five individuals has a single nucleotide polymorphism, rs12740374, in the 1p13 locus of the SORT1 gene that predisposes them to having low levels of low-density lipoprotein (LDL) and very-low-density lipoprotein (VLDL). Each copy of the minor allele, present in about 30% of people, alters LDL cholesterol by 8 mg/dL, while two copies of the minor allele, present in about 5% of the population, lowers LDL cholesterol 16 mg/dL. Carriers of the minor allele have also been shown to have a 40% decreased risk of myocardial infarction. Functional in vivo studies in mice describes that overexpression of SORT1 in mouse liver tissue led to significantly lower LDL-cholesterol levels, as much as 80% lower, and that silencing SORT1 increased LDL cholesterol approximately 200% (Musunuru K et al. From noncoding variant to phenotype via SORT1 at the 1p13 cholesterol locus. Nature 2010; 466:714-721).

### Modulation of Cell Fate.

Provided are methods of inducing an alteration in cell fate in a target mammalian cell. The target mammalian cell may be a precursor cell and the alteration may involve driving differentiation into a lineage, or blocking such differentiation. Alternatively, the target mammalian cell may be a differentiated cell, and the cell fate alteration includes driving de-differentiation into a pluripotent precursor cell, or blocking such de-differentiation, such as the dedifferentiation of cancer cells into cancer stem cells. In situations where a change in cell fate is desired, effective amounts of mRNAs encoding a cell fate inductive polypeptide is introduced into a target cell under conditions such that an alteration in cell fate is induced. In some embodiments, the modified mRNAs are useful to reprogram a subpopulation of cells from a first phenotype to a second phenotype. Such a reprogramming may be temporary or permanent. Optionally, the reprogramming induces a target cell to adopt an intermediate phenotype.

Additionally, the methods of the present invention are particularly useful to generate induced pluripotent stem cells (iPS cells) because of the high efficiency of transfection, the ability to re-transfect cells, and the tenability of the amount of recombinant polypeptides produced in the target cells. Further, the use of iPS cells generated using the methods described herein is expected to have a reduced incidence of teratoma formation.

Also provided are methods of reducing cellular differentiation in a target cell population. For example, a target cell population containing one or more precursor cell types is contacted with a composition having an effective amount of a modified mRNA encoding a polypeptide, under conditions such that the polypeptide is translated and reduces the differentiation of the precursor cell. In non-limiting embodiments, the target cell population contains injured tissue in a mammalian subject or tissue affected by a surgical procedure. The precursor cell is, e.g., a stromal precursor cell, a neural precursor cell, or a mesenchymal precursor cell.

In a specific embodiment, provided are modified nucleic acids that encode one or more differentiation factors Gata4, Mef2c and Tbx4. These mRNA-generated factors are introduced into fibroblasts and drive the reprogramming into cardiomyocytes. Such a reprogramming can be performed in vivo, by contacting an mRNA-containing patch or other material to damaged cardiac tissue to facilitate cardiac regeneration. Such a process promotes cardiomyocyte genesis as opposed to fibrosis.

### Targeting of Pathogenic Organisms; Purification of Biological Materials.

Provided herein are methods for targeting pathogenic microorganisms, such as bacteria, yeast, protozoa, helminthes and the like, using modified mRNAs that encode cytostatic or cytotoxic polypeptides. Preferably the mRNA introduced into the target pathogenic organism contains modified nucleosides or other nucleic acid sequence modifications that the mRNA is translated exclusively, or preferentially, in the target pathogenic organism, to reduce possible off-target effects of the therapeutic. Such methods are useful for removing pathogenic organisms from biological material, including blood, semen, eggs, and transplant materials including embryos, tissues, and organs.

### Targeting of Diseased Cells.

Provided herein are methods for targeting pathogenic or diseased cells, particularly cancer cells, using modified mRNAs that encode cytostatic or cytotoxic polypeptides. Preferably the mRNA introduced into the target pathogenic cell contains modified nucleosides or other nucleic acid sequence modifications that the mRNA is translated exclusively, or preferentially, in the target pathogenic cell, to reduce possible off-target effects of the therapeutic. Alternatively, the invention provides targeting moieties that are capable of targeting the modified mRNAs to preferentially bind to and enter the target pathogenic cell.

### Methods of Protein Production.

The methods provided herein are useful for enhancing protein product yield in a cell culture process. In a cell culture containing a plurality of host cells, introduction of the modified mRNAs described herein results in increased protein production efficiency relative to a corresponding unmodified nucleic acid. Such increased protein production efficiency can be demonstrated, e.g., by showing increased cell transfection, increased protein translation from the nucleic acid, decreased nucleic acid degradation, and/or reduced innate immune response of the host cell. Protein production can be measured by ELISA, and protein activity can be measured by various functional assays known in the art. The protein production may be generated in a continuous or a fed-batch mammalian process.

Additionally, it is useful to optimize the expression of a specific polypeptide in a cell line or collection of cell lines of potential interest, particularly an engineered protein such as a protein variant of a reference protein having a known activity. In one embodiment, provided is a method of optimizing expression of an engineered protein in a target cell, by providing a plurality of target cell types, and independently contacting with each of the plurality of target cell types a modified mRNA encoding an engineered polypeptide. Additionally, culture conditions may be altered to increase protein production efficiency. Subsequently, the presence and/or level of the engineered polypeptide in the plurality of target cell types is detected and/or quantitated, allowing for the optimization of an engineered polypeptide's expression by selection of an efficient target cell and cell culture conditions relating thereto. Such methods are particularly useful when the engineered polypeptide contains one or more post-translational modifications or has substantial tertiary structure, situations which often complicate efficient protein production.

### Methods of Gene Silencing.

The modified mRNAs described herein are useful to silence (i.e., prevent or substantially reduce) expression of one or more target genes in a cell population. A modified mRNA encoding a polypeptide capable of directing sequence-specific histone H3 methylation is introduced into the cells in the population under conditions such that the polypeptide is translated and reduces gene transcription of a target gene via histone H3 methylation and subsequent heterochromatin formation. In some embodiments, the silencing mechanism is performed on a cell population present in a mammalian subject. By way of non-limiting example, a useful target gene is a mutated Janus Kinase-2 family member, wherein the mammalian subject expresses the mutant target gene suffers from a myeloproliferative disease resulting from aberrant kinase activity.

Co-administration of modified mRNAs and siRNAs are also provided herein. As demonstrated in yeast, sequence-specific trans silencing is an effective mechanism for altering cell function. Fission yeast require two RNAi complexes for siRNA-mediated heterochromatin assembly: the RNA-induced transcriptional silencing (RITS) complex and the RNA-directed RNA polymerase complex (RDRC) (Motamedi et al. Cell 2004, 119, 789-802). In fission yeast, the RITS complex contains the siRNA binding Argonaute family protein Ago1, a chromodomain protein Chp1, and Tas3. The fission yeast RDRC complex is composed of an RNA-dependent RNA Polymerase Rdp1, a putative RNA helicase Hrr1, and a polyA polymerase family protein Cid12. These two complexes require the Dicer ribonuclease and Clr4 histone H3 methyltransferase for activity. Together, Ago1 binds siRNA molecules generated through Dicer-mediated cleavage of Rdp1 co-transcriptionally generated dsRNA transcripts and allows for the sequence-specific direct association of Chp1, Tas3, Hrr1, and Clr4 to regions of DNA destined for methylation and histone modification and subsequent compaction into transcriptionally silenced heterochromatin. While this mechanism functions in cis—with centromeric regions of DNA, sequence-specific trans silencing is possible through co-transfection with double-stranded siRNAs for specific regions of DNA and concomitant RNAi-directed silencing of the siRNA ribonuclease Eri1 (Buhler et al. Cell 2006, 125, 873-886).

### Modulation of Biological Pathways.

The rapid translation of modified mRNAs introduced into cells provides a desirable mechanism of modulating target biological pathways. Such modulation includes antagonism or agonism of a given pathway. In one embodiment, a method is provided for antagonizing a biological pathway in a cell by contacting the cell with an effective amount of a composition comprising a modified nucleic acid encoding a recombinant polypeptide, under conditions such that the nucleic acid is localized into the cell and the recombinant polypeptide is capable of being translated in the cell from the nucleic acid, wherein the recombinant polypeptide inhibits the activity of a polypeptide functional in the biological pathway. Exemplary biological pathways are those defective in an autoimmune or inflammatory disorder such as multiple sclerosis, rheumatoid arthritis, psoriasis, lupus erythematosus, ankylosing spondylitis colitis, or Crohn's disease; in particular, antagonism of the IL-12 and IL-23 signaling pathways are of particular utility. (See Kikly K, Liu L, Na S, Sedgwick J D (2006) Curr. Opin. Immunol. 18 (6): 670-5). Further, provided are modified nucleic acids encoding an antagonist for chemokine receptors; chemokine receptors CXCR-4 and CCR-5 are required for, e.g., HIV entry into host cells (Arenzana-Seisdedos F et al. (1996) Nature 383: 400).

Alternatively, provided are methods of agonizing a biological pathway in a cell by contacting the cell with an effective amount of a modified nucleic acid encoding a recombinant polypeptide under conditions such that the nucleic acid is localized into the cell and the recombinant polypeptide is capable of being translated in the cell from the nucleic acid, and the recombinant polypeptide induces the activity of a polypeptide functional in the biological pathway. Exemplary agonized biological pathways include pathways that modulate cell fate determination. Such agonization is reversible or, alternatively, irreversible.

### Methods of Cellular Nucleic Acid Delivery.

Methods of the present invention enhance nucleic acid delivery into a cell population, in vivo, ex vivo, or in culture. For example, a cell culture containing a plurality of host cells (e.g., eukaryotic cells such as yeast or mammalian cells) is contacted with a composition that contains an enhanced nucleic acid having at least one nucleoside modification and, optionally, a translatable region. The composition also generally contains a transfection reagent or other compound that increases the efficiency of enhanced nucleic acid uptake into the host cells. The enhanced nucleic acid exhibits enhanced retention in the cell population, relative to a corresponding unmodified nucleic acid. The retention of the enhanced nucleic acid is greater than the retention of the unmodified nucleic acid. In some embodiments, it is at least about 50%, 75%, 90%, 95%, 100%, 150%, 200% or more than 200% greater than the retention of the unmodified nucleic acid. Such retention advantage may be achieved by one round of transfection with the enhanced nucleic acid, or may be obtained following repeated rounds of transfection.

In some embodiments, the enhanced nucleic acid is delivered to a target cell population with one or more additional nucleic acids. Such delivery may be at the same time, or the enhanced nucleic acid is delivered prior to delivery of the one or more additional nucleic acids. The additional one or more nucleic acids may be modified nucleic acids or unmodified nucleic acids. It is understood that the initial presence of the enhanced nucleic acids does not substantially induce an innate immune response of the cell population and, moreover, that the innate immune response will not be activated by the later presence of the unmodified nucleic acids. In this regard, the enhanced nucleic acid may not itself contain a translatable region, if the protein desired to be present in the target cell population is translated from the unmodified nucleic acids.

### Pharmaceutical Compositions

The present invention provides enhanced nucleic acids, and complexes containing enhanced nucleic acids associated with other deliverable moieties. Thus, the present invention provides pharmaceutical compositions comprising one or more enhanced nucleic acids, or one or more such complexes, and one or more pharmaceutically acceptable excipients. Pharmaceutical compositions may optionally comprise one or more additional therapeutically active substances. In some embodiments, compositions are administered to humans. For the purposes of the present disclosure, the phrase “active ingredient” generally refers to an enhanced nucleic acid to be delivered as described herein.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100% (w/w) active ingredient.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro (Lippincott, Williams & Wilkins, Baltimore, MD, 2006; incorporated herein by reference) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween®20], polyoxyethylene sorbitan [Tween®60], polyoxyethylene sorbitan monooleate [Tween®80], sorbitan monopalmitate [Span®40], sorbitan monostearate [Span®60], sorbitan tristearate [Span®65], glyceryl monooleate, sorbitan monooleate [Span®80]), polyoxyethylene esters (e.g. polyoxyethylene monostearate [Myrj®45], polyoxyethylene hydrogenated castor oil, polyethoxylated castor oil, polyoxymethylene stearate, and Solutol®), sucrose fatty acid esters, polyethylene glycol fatty acid esters (e.g. Cremophor®), polyoxyethylene ethers, (e.g. polyoxyethylene lauryl ether [Brij®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic®F 68, Poloxamer®188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus®, Phenonip®, methylparaben, Germall®115, Germaben®II, Neolone™, Kathon™, and/or Euxyl®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient. Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

Dosage forms for topical and/or transdermal administration of a composition may include ointments, pastes, creams, lotions, gels, powders, solutions, sprays, inhalants and/or patches. Generally, an active ingredient is admixed under sterile conditions with a pharmaceutically acceptable excipient and/or any needed preservatives and/or buffers as may be required. Additionally, the present invention contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid compositions to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of active ingredient may be as high as the solubility limit of the active ingredient in the solvent. Formulations for topical administration may further comprise one or more of the additional ingredients described herein.

A pharmaceutical composition may be prepared, packaged, and/or sold in a formulation suitable for pulmonary administration via the buccal cavity. Such a formulation may comprise dry particles which comprise the active ingredient and which have a diameter in the range from about 0.5 nm to about 7 nm or from about 1 nm to about 6 nm. Such compositions are suitably in the form of dry powders for administration using a device comprising a dry powder reservoir to which a stream of propellant may be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (w/w) of the composition, and active ingredient may constitute 0.1% to 20% (w/w) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

A pharmaceutical composition may be prepared, packaged, and/or sold in a formulation suitable for ophthalmic administration. Such formulations may, for example, be in the form of eye drops including, for example, a 0.1/1.0% (w/w) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Ear drops and/or eye drops are contemplated as being within the scope of this invention.

General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference).

The present invention provides methods comprising administering modified mRNAs and their encoded proteins or complexes in accordance with the invention to a subject in need thereof. Nucleic acids, proteins or complexes, or pharmaceutical, imaging, diagnostic, or prophylactic compositions thereof, may be administered to a subject using any amount and any route of administration effective for preventing, treating, diagnosing, or imaging a disease, disorder, and/or condition (e.g., a disease, disorder, and/or condition relating to working memory deficits). The exact amount required will vary from subject to subject, depending on the species, age, and general condition of the subject, the severity of the disease, the particular composition, its mode of administration, its mode of activity, and the like. Compositions in accordance with the invention are typically formulated in dosage unit form for ease of administration and uniformity of dosage. It will be understood, however, that the total daily usage of the compositions of the present invention will be decided by the attending physician within the scope of sound medical judgment. The specific therapeutically effective, prophylactially effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

### Kits.

The invention provides a variety of kits for conveniently and/or effectively carrying out methods of the present invention. Typically kits will comprise sufficient amounts and/or numbers of components to allow a user to perform multiple treatments of a subject(s) and/or to perform multiple experiments.

### Definitions

Therapeutic Agent: The term “therapeutic agent” refers to any agent that, when administered to a subject, has a therapeutic, diagnostic, and/or prophylactic effect and/or elicits a desired biological and/or pharmacological effect.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, where a nucleic acid is biologically active, a portion of that nucleic acid that shares at least one biological activity of the whole nucleic acid is typically referred to as a “biologically active” portion.

Conserved: As used herein, the term “conserved” refers to nucleotides or amino acid residues of a polynucleotide sequence or amino acid sequence, respectively, that are those that occur unaltered in the same position of two or more related sequences being compared. Nucleotides or amino acids that are relatively conserved are those that are conserved amongst more related sequences than nucleotides or amino acids appearing elsewhere in the sequences. In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Functional: As used herein, a “functional” biological molecule is a biological molecule in a form in which it exhibits a property and/or activity by which it is characterized.

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical. In some embodiments, polymeric molecules are considered to be “homologous” to one another if their sequences are at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% similar. The term “homologous” necessarily refers to a comparison between at least two sequences (nucleotides sequences or amino acid sequences). In accordance with the invention, two nucleotide sequences are considered to be homologous if the polypeptides they encode are at least about 50% identical, at least about 60% identical, at least about 70% identical, at least about 80% identical, or at least about 90% identical for at least one stretch of at least about 20 amino acids. In some embodiments, homologous nucleotide sequences are characterized by the ability to encode a stretch of at least 4-5 uniquely specified amino acids. Both the identity and the approximate spacing of these amino acids relative to one another must be considered for nucleotide sequences to be considered homologous. For nucleotide sequences less than 60 nucleotides in length, homology is determined by the ability to encode a stretch of at least 4-5 uniquely specified amino acids. In accordance with the invention, two protein sequences are considered to be homologous if the proteins are at least about 50% identical, at least about 60% identical, at least about 70% identical, at least about 80% identical, or at least about 90% identical for at least one stretch of at least about 20 amino acids.

Identity: As used herein, the term “identity” refers to the overall relatedness between polymeric molecules, e.g., between nucleic acid molecules (e.g. DNA molecules and/or RNA molecules) and/or between polypeptide molecules. Calculation of the percent identity of two nucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12 (1), 387 (1984)), BLASTP, BLASTN, and FASTA Atschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically a reduction in the level of an mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe).

Isolated: As used herein, the term “isolated” refers to a substance or entity that has been (1) separated from at least some of the components with which it was associated when initially produced (whether in nature or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Similarity: As used herein, the term “similarity” refers to the overall relatedness between polymeric molecules, e.g. between nucleic acid molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Subject: As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to a disease, disorder, and/or condition, to treat, improve symptoms of, diagnose, prevent, and/or delay the onset of the disease, disorder, and/or condition.

Transcription factor: As used herein, the term “transcription factor” refers to a DNA-binding protein that regulates transcription of DNA into RNA, for example, by activation or repression of transcription. Some transcription factors effect regulation of transcription alone, while others act in concert with other proteins. Some transcription factor can both activate and repress transcription under certain conditions. In general, transcription factors bind a specific target sequence or sequences highly similar to a specific consensus sequence in a regulatory region of a target gene. Transcription factors may regulate transcription of a target gene alone or in a complex with other molecules.

Treating: As used herein, the term “treating” refers to partially or completely alleviating, ameliorating, improving, relieving, delaying onset of, inhibiting progression of, reducing severity of, and/or reducing incidence of one or more symptoms or features of a particular disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to the protein or agent prior to being modified.

### Equivalents and Scope

Those skilled in the art will recognize, or be able to ascertain using no more than routine experimentation, many equivalents to the specific embodiments, described herein. The scope of the present invention is not intended to be limited to the above Description, but rather is as set forth in the appended claims.

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

Modified mRNAs (mmRNAs) according to the invention were made using standard laboratory methods and materials. The open reading frame (ORF) of the gene of interest is flanked by a 5′ untranslated region (UTR) containing a strong Kozak translational initiation signal and an alpha-globin 3′ UTR terminating with an oligo (dT) sequence for templated addition of a polyA tail. The mmRNAs were modified with pseudouridine (w) and 5-methyl-cytidine (5meC) to reduce the cellular innate immune response. Kariko K et al. Immunity 23:165-75 (2005), Kariko K et al. Mol Ther 16:1833-40 (2008), Anderson B R et al. NAR (2010).

The cloning, gene synthesis and vector sequencing was performed by DNA2.0 Inc. (Menlo Park, CA). Vector sequences and insert sequences are set forth in SEQ ID NOs: 5-8. The ORFs were restriction digested using XbaI or HindIII and used for cDNA synthesis using tailed-PCR. This tailed-PCR cDNA product was used as the template for the modified mRNA synthesis reaction using 25 mM each modified nucleotide mix (modified U/C was manufactured by TriLink Biotech, San Diego, CA, unmodifed A/G was purchased from Epicenter Biotechnologies, Madison, WI) and CellScript MegaScript™ (Epicenter Biotechnologies, Madison, WI) complete mRNA synthesis kit. The in vitro transcription reaction was run for 3-4 hours at 37° C. PCR reaction used HiFi PCR 2× Master Mix™ (Kapa Biosystems, Woburn, MA). The In vitro transcribed mRNA product was run on an agarose gel and visualized. mRNA was purified with Ambion/Applied Biosystems (Austin, TX) MEGAClear RNA™ purification kit. PCR used PureLink™ PCR purification kit (Invitrogen, Carlsbad, CA) or PCR cleanup kit (Qiagen, Valencia, CA). The product was quantified on Nanodrop™ UV Absorbance (ThermoFisher, Waltham, MA). Quality, UV absorbance quality and visualization of the product was performed on an 1.2% agarose gel. The product was resuspended in TE buffer.

When transfected into mammalian cells, the modified mRNAs may have a stability of between 12-18 hours.

For animal experiments, the IV delivery solution was 150 mM NaCl, 2 mM CaCl2, 2 mM Na+-phosphate, and 0.5 mM EDTA, pH 6.5 and 10 μl lipofectamine (RNAiMax™, Invitrogen, Carlsbad, CA).

### Example 1

The nucleic acid sequence for the precursor of human granulocyte colony stimulating
factor (G-CSF) is set forth in SEQ ID NO. 1:
(SEQ ID No. 1)
agcttttggaccctcgtacagaagctaatacgactcactatagggaaataagagagaaaagaagagtaagaagaaatataagagccaccat
ggccggtcccgcgacccaaagccccatgaaacttatggccctgcagttgctgctttggcacteggccctctggacagtccaagaagcgact
cctctcggacctgcctcatcgttgccgcagtcattccttttgaagtgtctggagcaggtgcgaaagattcagggcgatggagccgcactcca
agagaagctctgcgcgacatacaaactttgccatcccgaggagctcgtactgctcgggcacagcttggggattccctgggctcctctctcgt
cctgtccgtcgcaggctttgcagttggcagggtgcctttcccagctccactccggtttgttcttgtatcagggactgctgcaagcccttgag
ggaatctcgccagaattgggcccgacgctggacacgttgcagctcgacgtggcggatttcgcaacaaccatctggcagcagatggaggaact
ggggatggcacccgcgctgcagcccacgcagggggcaatgccggcctttgcgtccgcgtttcagcgcagggcgggtggagtcctcgta
gcgagccaccttcaatcatttttggaagtctcgtaccgggtgctgagacatcttgcgcagccgtgaagcgctgccttctgcggggcttgcct
tctggccatgcccttcttctctcccttgcacctgtacctcttggtctttgaataaagcctgagtaggaaggcggccgctcgagcatgcatct
agagggcccaattcgccctattcgaagtcg
The nucleic acid sequence for G-CSF mRNA is set forth in SEQ ID NO. 17:
(SEQ ID No. 17)
agcuuuuggacccucguacagaagcuaauacgacucacuauagggaaauaagagagaaaagaagaguaagaagaaauauaaga
gccaccauggccggucccgcgacccaaagccccaugaaacuuauggcccugcaguugcugcuuuggcacucggcccucuggac
aguccaagaagcgacuccucucggaccugccucaucguugccgcagucauuccuuuugaagugucuggagcaggugcgaaag
auucagggcgauggagccgcacuccaagagaagcucugcgcgacauacaaacuuugccaucccgaggagcucguacugcucgg
gcacagcuuggggauucccugggcuccucucucguccuguccgucgcaggcuuugcaguuggcagggugccuuucccagcu
ccacuccgguuuguucuuguaucagggacugcugcaagcccuugagggaaucucgccagaauugggcccgacgcuggacacg
uugcagcucgacguggcggauuucgcaacaaccaucuggcagcagauggaggaacuggggauggcaccegcgcugcagccca
cgcagggggcaaugccggccuuugcguccgcguuucagcgcagggcggguggaguccucguagcgagccaccuucaaucauu
uuuggaagucucguaccgggugcugagacaucuugcgcagccgugaagcgcugccuucugggggcuugccucuggccau
gcccuucuucucucccuugcaccuguaccucuuggucuuugaaaaagccugaguaggaaggggccgcucgagcaugcauc
uagagggcccaauucgcccuauucgaagucg
The nucleic acid sequence for an exemplary G-CSF modified mRNA (mmRNA) is
set forth in SEQ ID NO. 18:
(SEQ ID No. 18)
ag5meCψψψψgga5meC5meC5meCψ5meCgψa5meCagaag5meCψaaψa5meCga5meCψ5meCa5
meCψaψagggaaaψaagagagaaaagaagagψaagaagaaaψaψaagag5meC5meCa5meC5meCaψgg5meC
5meCggψ5meC5meC5meCg5meCga5meC5meC5meCaaag5meC5meC5meC5meCaψgaaa5meC
ψψaψgg5meC5meC5meCψg5meCagψψg5meCψg5meCψψψgg5meCa5meCψ5meCgg5meC5me
C5meCψ5meCψgga5meCagψ5meC5meCaagaag5meCga5meCψ5meC5meCψ5meCψ5meCgga5
meC5meCψg5meC5meCψ5meCaψ5meCgψψg5meC5meCg5meCagψ5meCaψψ5meC5meCψψψ
ψgaagψgψ5meCψggag5meCaggψg5meCgaaagaψψ5meCaggg5meCgaψggag5meC5meCg5meCa
5meCψ5meC5meCaagagaag5meCψ5meCψg5meCg5meCga5meCaψa5meCaaa5meCψψψg5me
C5meCaψ5meC5meC5meCgaggag5meCψ5meCgψa5meCψg5meCψ5meCggg5meCa5meCag5
meCψψggggaψψ5meC5meC5meCψggg5meCψ5meC5meCψ5meCψ5meCψ5meCgψ5meC5meC
ψgψ5meC5meCgψ5meCg5meCagg5meCψψψg5meCagψψgg5meCagggψg5meC5meCψψψ5me
C5meC5meCag5meCψ5meC5meCa5meCψ5meC5meCggψψψgψψ5meCψψgψaψ5meCaggga5m
eCψg5meCψg5meCaag5meC5meC5meCψψgagggaaψ5meCψ5meCg5meC5meCagaaψψggg5me
C5meC5meCga5meCg5meCψgga5meCa5meCgψψg5meCag5meCψ5meCga5meCgψgg5meCgg
aψψψ5meCg5meCaa5meCaa5meC5meCaψ5meCψgg5meCag5meCagaψggaggaa5meCψggggaψ
gg5meCa5meC5meC5meCg5meCg5meCψg5meCag5meC5meC5meCa5meCg5meCaggggg5me
Caaψg5meC5meCgg5meC5meCψψψg5meCgψ5meC5meCg5meCgψψψ5meCag5meCg5meCag
gg5meCgggψggagψ5meC5meCψ5meCgψag5meCgag5meC5meCa5meC5meCψψ5meCaaψ5me
Cavψψψψggaagψ5meCψ5meCgψa5meC5meCgggψg5meCψgaga5meCaψ5meCψψg5meCg5me
Cag5meC5meCgψgaag5meCg5meCψg5meC5meCψψ5meCψg5meCgggg5meCψψg5meC5meC
ψψ5meCψgg5meC5meCaψg5meC5meC5meCψψ5meCψψ5meCψ5meCψ5meC5meC5meCψψg
5meCa5meC5meCψgψa5meC5meCψ5meCψψggψ5meCψψψgaaψaaag5meC5meCψgagψaggaag
g5meCgg5meC5meCg5meCψ5meCgag5meCaψg5meCaψ5meCψagaggg5meC5meC5meCaaψψ
5meCg5meC5meC5meCψaψψ5meCgaagψ5meCg

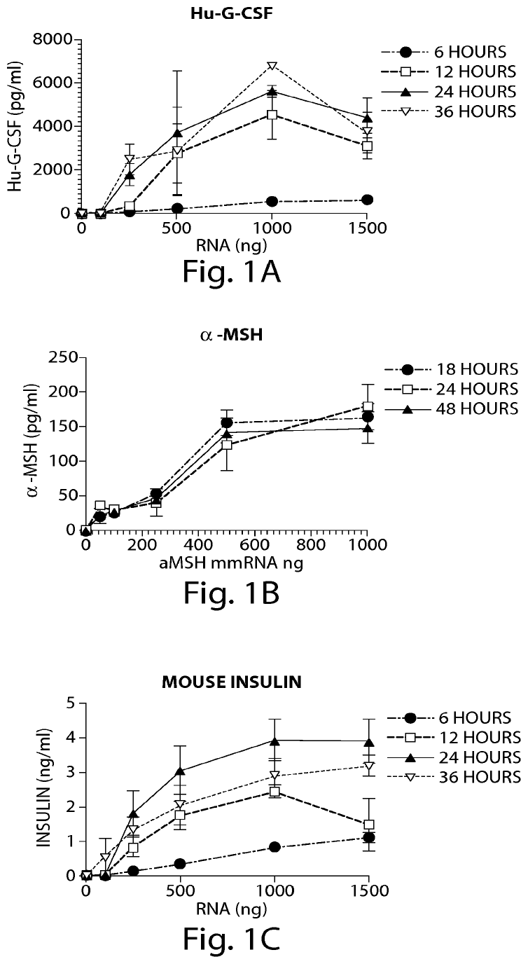

FIG. 1a shows an Enzyme-linked immunosorbent assay (ELISA) for Human Granulocyte-Colony Stimulating Factor (G-CSF) of in vitro transfected Human Keratinocyte cells. Keratinocytes were grown in EpiLife medium with Supplement S7 from Invitrogen until they reached a confluence of 50-70%. Cells were transfected with 0, 100, 250, 500, 1000, and 1500 ng mmRNA complexed with RNAiMAX from Invitrogen. The RNA: RNAiMAX complex was formed by first incubating the RNA with Supplement-free EpiLife media in a 5× volumetric dilution for 10 minutes at room temperature. In a second vial, RNAiMAX reagent was incubated with Supplement-free EpiLife Media in a 10× volumetric dilution for 10 minutes at room temperature. The RNA vial was then mixed with the RNAiMAX vial and incubated for 20-30 at room temperature before being added to the cells in a drop-wise fashion. Secreted huG-CSF concentration in the culture medium was measured at 6, 12, 24, and 36 hours post-transfection for each of the 6 concentrations. Secretion of Human Granulocyte-Colony Stimulating Factor (G-CSF) from transfected human keratinocytes was quantified using an ELISA kit from Invitrogen following the manufacturers recommended instructions. HuG-CSF shows a dose response, with 1000 ng mmRNA showing the best response. Also, production peaked between 24 and 36 hours after transfection. These data show that huG-CSF mmRNA (SEQ ID NO: 1) is capable of being translated in Human Keratinocyte cells and that huG-CSF is transported out of the cells and released into the extracellular environment.

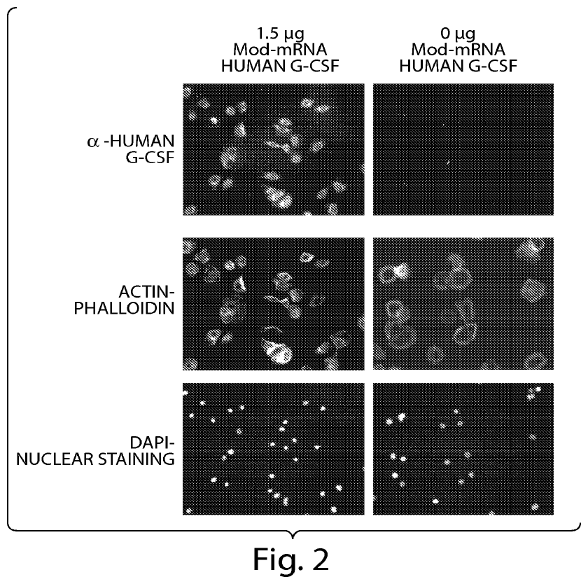

FIG. 2 shows human keratinocytes transfected with 0 or 1.5 μg G-CSF mmRNA. Cells were grown in EpiLife Media with Supplement S7 from Invitrogen according to standard protocols in 24-well collagen-coated plates. Cells were fixed with 4% paraformaldehyde in PBS and permeabilized with 0.1% Triton X-100 in PBS for 5-10 minutes at room temperature. Cells were then washed 3× with room temperature PBS. G-CSF protein staining was performed using G-CSF mouse monoclonal antibody 5D7 (ab9818, abcam, Cambridge, MA) and goat polyclonal secondary antibody to mouse IgG conjugated to DyLight® 594 (ab97017, abcam) according to the manufacturer's recommended dilutions. Nuclear DNA staining was performed with DAPI dye from Invitrogen. The cellular actin cytoskeleton was stained using AlexaFluor488-conjugated phalloidin from Invitrogen. G-CSF protein is translated and localized to the cytoplasm upon G-CSF mmRNA transfection. The pictures were taken 36 hours after transfection.

Mice (C57 BL/6NTac males approx. 8 weeks old, group housed) were intravenously (IV) injected in the tail vein (Dosing regimen: once daily on days 0, 2, and 4) with human G-CSF mmRNA (SEQ ID No. 1) and complete blood cell count with full differential following mmRNA injection was measured. Group size: N=5 for each treatment group and time of bleeding.

### Experimental Design

| mmRNA | Unmodified mRNA | Control | Control | |
|---|---|---|---|---|
| hpreG-CSF | hpreG-CSF | Scramble | Vehicle | Number of |
| IV Tail vein | IV Tail vein | IV Tail vein | IV Tail vein | animals |
| 1 day (n = 5) | 1 day (n = 5) | 1 day (n = 5) | 1 day(n = 5) | 20 (squad A) |
| 5 days (n = 5) | 5 days (n = 5) | 5 days (n = 5) | 5 days (n = 5) | 20 (squad B) |
| 8 days (n = 5) | 8 days (n = 5) | 8 days (n = 5) | 8 days(n = 5) | squad A |
| 14 days (n = 5) | 14 days (n = 5) | 14 days (n = 5) | 14 days (n = 5) | squad B |
| 20 days (n = 5) | 20 days (n = 5) | 20 days (n = 5) | 20 days (n = 5) | Squad A |
| Total Mice | | | | 40 |

### Treatment Groups:

mmRNA hpreG-CSF
unmodified hpreG-CSF
Control: non-specific RNA (total yeast RNA)
Control: vehicle

### Diet:

Regular chow diet: Pico Rodent Chow (5053)

### Assay:

Whole blood for hematological analysis was collected, and the following cells were counted: Total white blood cells, neutrophils, lymphocytes, monocytes/macrophages, eosinophils, basophils, red blood cells, platelets. Body weights were measured during blood collection.

### Results

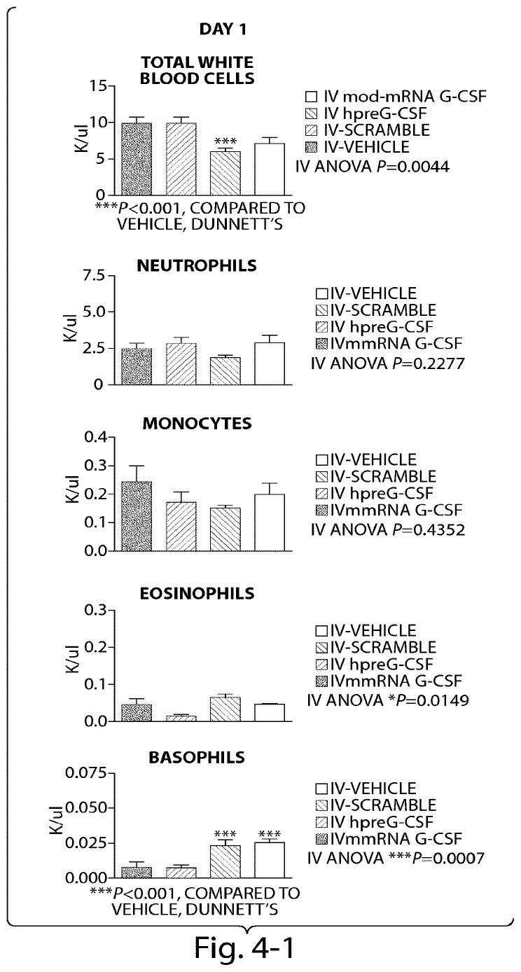

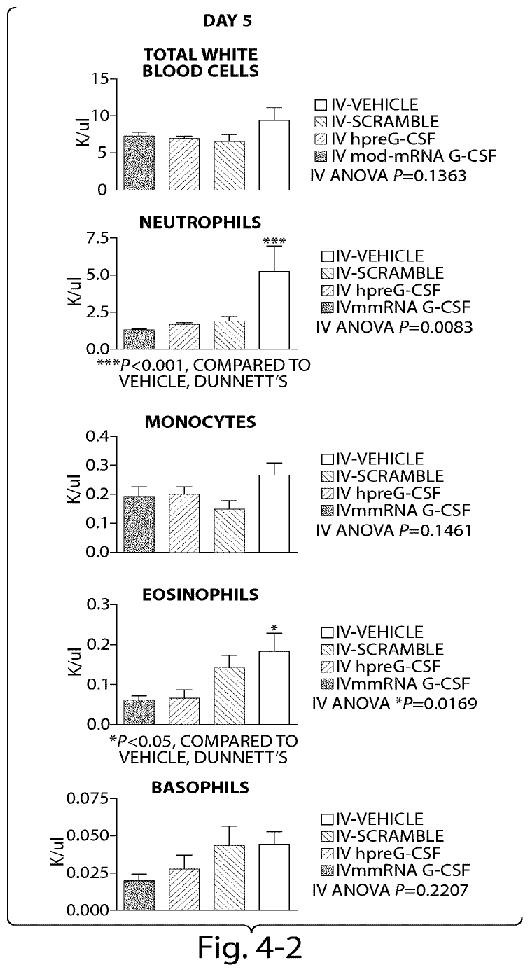

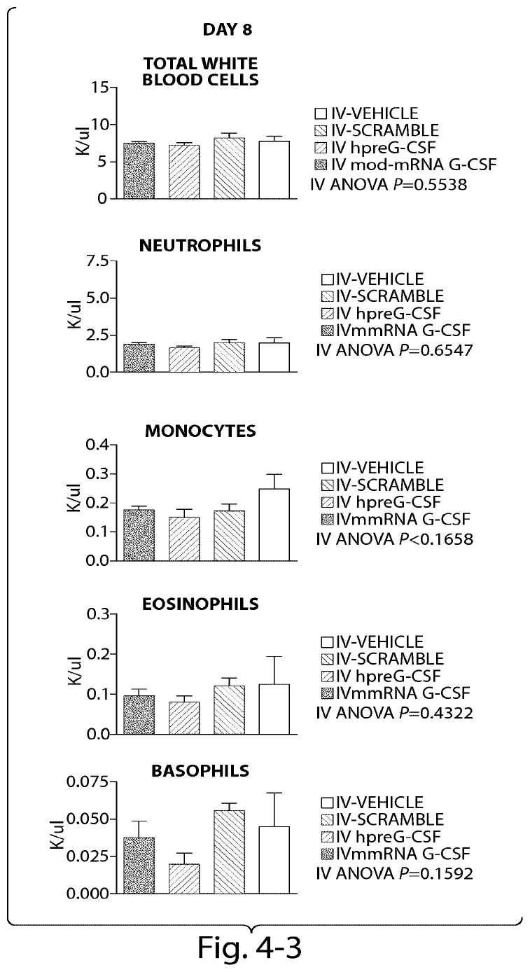

FIG. 4 shows the results for in vivo activity of huG-CSF mmRNA on total blood counts and granulocytes. Row 1 shows the total white blood cell (WBC) count. huG-CSF mmRNA alone elevated the total WBC count on day 5 after administration by IV. In contrast, non-modified hu-G-CSF, vehicle alone, non-specific mRNA (scramble: total yeast RNA) did not alter WBC counts. G-CSF is known to stimulate the proliferation, differentiation and mobilization of myeloid progenitor cells including terminally differentiated granulocyte populations of neutrophil, eosinophils, monocytes, and basophils. G-CSF does not stimulate lymphoid or erythrocyte cell types including lymphocytes, red blood cells or platelets. Row 2 shows the neutrophil count. G-CSF is known to particularly stimulate neutrophil proliferation, mobilization and progenitor differentiation. Again, only huG-CSF mmRNA, but not vehicle alone, non-specific mRNA (scramble: total yeast RNA) or non-modified huG-CSF mRNA elevated neutrophils 4-fold above vehicle control on day 5 after administration by IV. Additional granulocyte cell types in Rows 3 and 5 show slight elevation of monocytes and basophils at days 5 and 8 for animals treated with huG-CSF mmRNA, while Row 4 shows significant upregulation of the granulocyte lineage eosinophils on day 5.

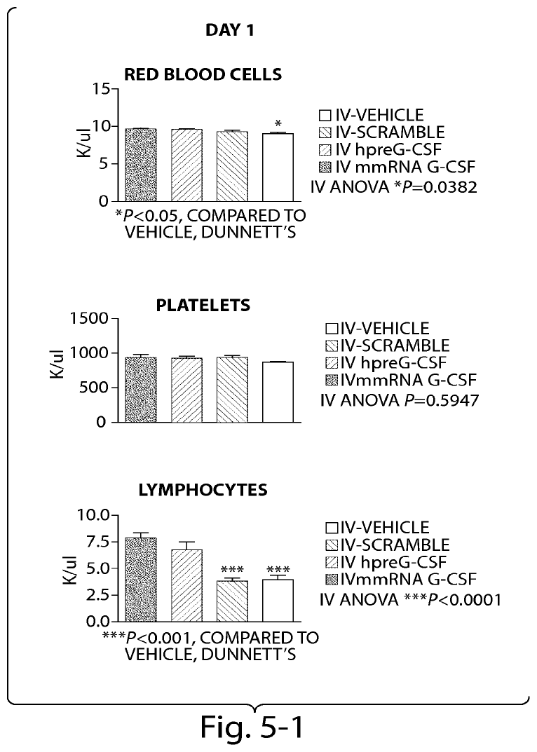

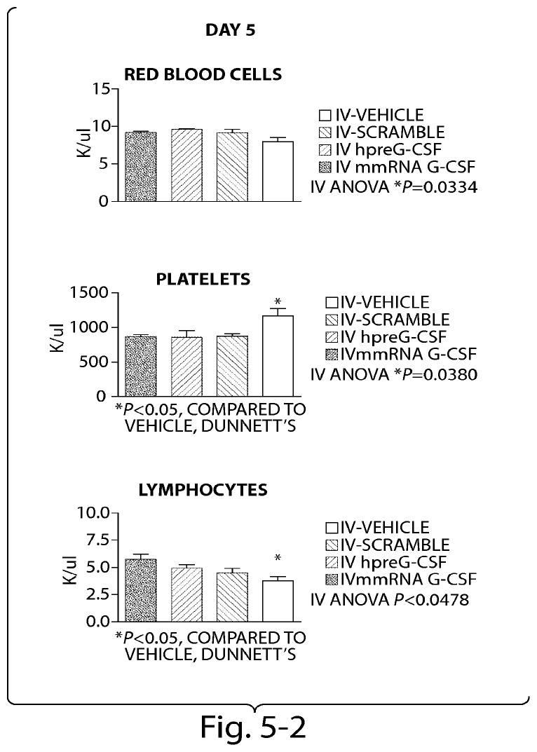

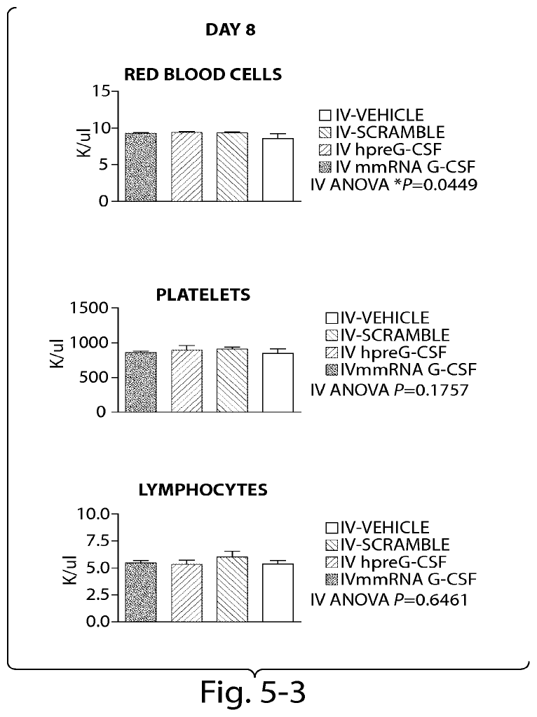

FIG. 5 shows the results for in vivo activity of huG-CSF mmRNA on erythrocyte and lymphocyte cell types. Row 1 shows that the red blood cell (RBC) count stays relative stable, which is expected. Row 2 shows platelet count. Row 3 shows the lymphocyte count, which remains stable across all treatment groups as expected. Together, these data shows that huG-CSF mmRNA is translated in mouse cells in vivo, correctly processed and released. The G-CSF precursor form contains a signal peptide that must be cleaved for the protein to be secreted. G-CSF contains the human signal peptide not that of the mouse. It appears that the human signal peptide was correctly processed and led huG-CSF to be released from the transfected cells resulting the pronounced physiological response. huG-CSF peptide made from mmRNA was active in vivo. huG-CSF elevated total WBC count and stimulated each granulocyte cell type (neutrophil, monocytes, eosinophils, basophils) but not non-granulocyte populations.

### Example 2

The effects of modified mRNA encoding G-CSF on stimulating polymorphonuclear neutrophil (PMN) and modulating PMN functions are tested using G-CSFR-deficient mice according to Betsuyacu T et al. J Clin Invest 103:825-832 (1999). G-CSFR-deficient mice (C57BL/6×129 SvJ) are generated as described in Liu F et al. Immunity 5:491-501 (1996). The mice carry a homozygous null mutation in the granulocyte colony-stimulating factor receptor (G-CSFR) gene. G-CSFR-deficient mice show decreased numbers of normal circulating neutrophils, hematopoietic progenitors are decreased in the bone marrow, and the expansion and terminal differentiation of these progenitors into granulocytes is impaired. Neutrophils isolated from G-CSFR-deficient mice are prone to apoptosis. Six- to 10-week-old wild-type and G-CSFR-deficient mice may be used for studies. PMNs can be purified from bone marrow of mice using a discontinuous Percol gradient, e.g. to reach 40%-60% purity.

### Example 3

The effects of modified mRNA encoding G-CSF are tested using C57BL/6 mice with homozygous inactivation of the G-CSF gene (G-CSF−/−) according to Lieschke G J et al. Blood 84:1737 (1994). Mice are analyzed between 2 and 3 months of age. To induce an acute neutrophil inflammatory response, mice are injected intraperitoneal (IP) with a preparation comprising casein containing bacteria, e.g. 2 mL of an 0.2% (wt/vol) solution of casein in mouse tonicity phosphate-buffered saline (MTPBS), using either calcium caseinate, casein C5890 (Sigma Chemical Co, St Louis, MO), casein 44016, casein C3400, casein hydrolysate L41, casein sodium 2330. In other experiments, peritoneal inflammatory responses are induced by the IP injection of 2 mL of 0.3% sodium thioglycollate (wt/vol in distilled water) according to Metcalf D et al. Blood 88:3755-3764 (1996). White blood cell counts are performed under anesthesia on orbital plexus blood before and, on the opposite eye, 3 hours after the IP injections. At 3 hours after injection, blood is collected from the axilla during anesthesia-induced killing. The abdominal cavity is then injected with 2 mL MTPBS and massaged to ensure adequate mixing of the cell population with the harvesting fluid. The peritoneal cavity cells are then collected. The marrow cells are collected from one femur using 2 mL of medium. The marrow plug is converted to a dispersed cell suspension. Total marrow cell counts are calculated. The spleen is weighed and converted to a dispersed cell suspension. Mice are injected with modified mRNA, recombinant G-CSF (rhG-CSF) vehicle control and unspecific mRNA control. For recombinant G-CSF 2.5 pg (in 0.2 mL of 0.9% saline containing 5% bovine calf serum) is injected subcutaneously twice daily for 5 days. A 2.5-day period is then allowed to elapse before the mice are injected IP with MTPBS or casein. Control mice are injected twice daily with 0.2 mL MTPBS containing 5% FCS (vehicle control).

### Example 4

Delivery of modified mRNA encoding G-CSF is tested in female ICR mice (e.g. two months old). For skin injection, to mimic a surgical wound, mice are anesthetized, their backs shaved and disinfected with 70% ethanol, and then a 1 cm long, full thickness surgical incision is made at individual sites on the dorsum of the back of each animal. Immediately thereafter, various concentrations of modified mRNA either uncomplexed (naked) or complexed (e.g. liposome) are injected into the wound edges of each of the wound sites on each animal. Wounds are closed using a metal clip. For intravenous injection (e.g. tail vein), mice each receive various concentrations of modified mRNA either uncomplexed (naked) or complexed. The mice are killed and skin samples as well as liver, spleen, heart, lungs, and lymph nodes were harvested at 0 (control), 1, 3, 6, and 24 h and 1, 2, 5, and 8 wk after injection, according to Meuli M et al. J Invest Dermatology 116, 131-135 (2001).

### Example 5

The effects of modified mRNA encoding G-CSF on cerebral ischemia are tested using adult male Sprague-Dawley rats (weight, 250 to 300 g). One day after induction of cerebral ischemia, rats are injected subcutaneously with modified mRNA encoding G-CSF or recombinant human G-CSF (50 μg/kg per day; Amgen Biologicals) once daily for 5 days. Control animals are subjected to cerebral ischemia and injected with saline, according to Shyu WC Circulation 110:1847-1854 (2004). Under anesthesia, ligations of the right middle cerebral artery (MCA) and bilateral common carotid arteries (CCAs) are performed to induce cerebral infarction. Bilateral CCAs are clamped with nontraumatic arterial clips. With the use of a surgical microscope, the right MCA is ligated with a 10-0 nylon suture. Cortical blood flow is measured continuously with a laser-Doppler flowmeter (PF-5010, Periflux system, Perimed AB) in anesthetized animals. After 90 minutes of ischemia, the suture on the MCA and arterial clips on CCAs are removed to allow reperfusion. During recovery from the anesthesia, body temperature is maintained at 37° C. with a heat lamp. Bromodeoxyuridine (BrdU) pulse labeling is performed to observe the time course of proliferative cells in the brain after cerebral ischemia. G-CSF-treated rats and control rats are injected intraperitoneally with BrdU (50 mg/kg) every 4 hours for 12 hours before they are killed. Rats are killed at 7 days, 14 days, and 28 days after cerebral ischemia. A cumulative labeling method is used to examine the population of proliferative cells during 14 days of cerebral ischemia. Rats G-CSF-treated rats and control rats receive daily injections of BrdU (50 mg/kg IP) for 14 consecutive days, starting the day after MCA ligation. These rats are euthanized 14 days after the last injection. Behavioral assessments are performed 5 days before cerebral ischemia and 1, 7, 14, and 28 days subsequent to MCA ligation. The tests measure (1) body asymmetry and (2) locomotor activity. The baseline-tested scores are recorded to normalize those taken after cerebral ischemia. (1) The elevated body swing test is used to assess body asymmetry after MCA ligation and is evaluated quantitatively. (2) For locomotor activity, rats are subjected to OPTO-VARIMAX (Columbus Instruments) activity monitoring for about 2 hours for behavioral recording. Motor activity is counted as the number of beams broken by rat movement in the chamber. Two parameters of vertical movement are calculated: (1) vertical activity and (2) vertical time. MRI is performed in an imaging system (General Electric) at 3.0 T. Under anesthesia, the 6 to 8 coronal image slices are each 2 mm thick without any gaps. T2-weighted imaging (T2WI) pulse sequences are obtained with the use of a spin-echo technique (repetition time, 4000 ms; echo time, 105 ms) and are captured sequentially for each animal at 1, 7, and 28 days after cerebral ischemia. To measure the infarction area in the right cortex, the non-infarcted area in the right cortex is subtracted from the total cortical area of the left hemisphere. The brains of experimental rats are fixed by transcardial perfusion with saline, followed by perfusion and immersion in 4% paraformaldehyde, followed by BrdU immunostaining. Quantification of BrdU-immunoreactive cells is performed on paraffin-embedded tissue sections and is counted digitally. Shyu W C Circulation 110:1847-1854 (2004).

### Example 6

The effects of modified mRNA encoding G-CSF on thromboembolic cerebral ischemia (TE) are tested using a rat model of TE of male Wistar rats according to Kollmar R Exp & Transl Stroke Med 2:9 (2010). Intravenous administration of modified mRNA encoding G-CSF follows TE after 60 minutes or 180 minutes. TE is induced by exposing the right common carotid (CCA), internal carotid (ICA), and external carotid artery (ECA) and further dissection to identify the origin of the pterygopalatine artery (PPA). The ECA and the PPA are permanently ligated while the CCA is only temporarily clipped for embolization. A PE 50 catheter is inserted into the ECA proximal to its ligation and 12 red blood clots (each 0.35 mm in diameter and 3 mm in length) are injected at the origin of the right middle cerebral artery (MCA). All animals are subjected to MRI monitoring including perfusion weighted imaging (PWI), diffusion weighted imaging (DWI), T2, and T2* at 0.5, 2.5, 4, and 24 hours after TE followed by silver-infarct staining (SIS). All surviving animal are tested for neurological outcome: no apparent deficit, contralateral forelimb flexion; decreased grip of contralateral forelimb grip while tail pulled; spontaneous movement in all directions, contralateral circling only if pulled by tail; spontaneous contralateral circling.

### Example 7

The effects of modified mRNA encoding G-CSF on Alzheimer's disease is tested in an AD mouse model according to Tsai K J et al. J Exp Med. 204:1273-80 (2007). The acute Aβ-induced model is generated according to Stephan A et al. J. Neurosci. 21:5703-5714 (2001) and Yan J J et al. Br. J. Pharmacol. 133:89-96 (2001) using 8-wk-old C57BL/6 male mice. The Aβ aggregate is prepared from a solution of 10 mM of soluble Aβ₍₁₋₄₂₎ (Sigma-Aldrich) in 0.01 M PBS, pH 7.4. The solution is incubated at 37° C. for 3 days to form the aggregated Aβ and stored at −70° C. Animals are intraperitoneally anesthetized injected with aggregated Aβ bilaterally into the dorsal hippocampus. The animals are subjected to stereotaxic surgery with the incisor bar set at the following coordinates: 2 mm posterior to the bregma, 2.1 mm bilateral to the midline, and 1.8 mm ventral to the skull surface. The volume of injection is 1 μl of aggregated Aβ or 1 μl PBS, and 7 days are allowed for AD symptoms to develop in the mice. For the chronic AD model, Tg2576 mice are purchased from Taconic (Hudson, NY). For the acute AD model, 7 days after injection of the aggregated Aβ, mice are subcutaneously injected with various concentrations of modified mRNA encoding G-CSF and as a control with either 50 μg/kg of recombinant human G-CSF (Amgen Biologicals) or carrier (PBS) once daily for 5 consecutive days. Tg2576 mice are treated similarly. Behavioral measurements are conducted, using e.g. the Morris water maze learning task for spatial learning. Acetycholin levels are measured in the mouse brains. The mice are killed, and their brains are quickly removed and frozen on dry ice. The brains are homogenized on ice and subjected to the ACh assay, e.g. the Amplex Red Acetylcholine/Acetylcholinesterase Assay Kit (Invitrogen), according to the manufacturer's instructions. Quantifications of the AB levels and AB plaque burden are carried out measuring the levels of soluble and insoluble Aβ were quantified according to the procedures of Kawarabayashi T et al. J. Neurosci. 21:372-381 (2001) and Janus C et al. Nature. 408:979-982 (2000).

### Example 8

The effects of modified mRNA encoding G-CSF on improved cardiac function after myocardial infarction (MI) its anti-apoptotic effects on cardiomyocytes, as well as its role in collateral artery growth (arteriogenesis), can be tested using a model of MI according to Deindl E et al. FASEB J 20:956-958 (2006). MI is induced in male C57BL/6 mice 8-12 wk of age by surgical occlusion of the left anterior descending artery (LAD) through a left anterolateral approach. Mice are anesthetized by intraperitoneal (ip) injection of a mixture of 100 mg/kg ketamine and 5 mg/kg Xylazine, intubated, and artificially ventilated by a mouse ventilator with 200 strokes/min and 200 μl/stroke. Mice are treated with various concentrations of modified mRNA encoding G-CSF and as a control with recombinant G-CSF (100 μg/kg/day, subcutaneous s.c., Amgen Biologicals) directly after MI for 5 consecutive days. Additional controls are sham-operated animals and not operated animals receiving saline. All animals receive bromodeoxyuridine (BrdU; 50 μg/kg/day for 5 consecutive days). BrdU and cytokine treatment is started 30 min after ligation of the LAD. Peripheral blood is harvested from each mouse by aspirating the carotid artery. To define the number of leukocytes, heparinized blood samples are analyzed using a hematological cell analyzer. Mononuclear cells are separated by density-gradient centrifugation, purified, and resuspended in PBS containing 1% BSA. Cells are stained with various markers and are analyzed using FACS. Hearts are excised, fixed, cut transversally into 2 mm thick slices, processed, and embedded in paraffin by standard methods; 4 μm thick sections are cut and mounted on positively charged glass slides. Standard histological procedures (hematoxylin/eosin and Masson trichrome) and immunostaining (see below) are performed. Infarct size is determined as area of infarction (AI) correlated to the area of the left ventricle (including LV-septum). Wall thickness is also measured. Pressure-volume relationships in vivo are analyzed on surviving mice.

### Example 9

The effects of modified mRNA encoding G-CSF on autoimmune disease is tested using an EAE mouse model according to Zavala F et al. J Immunol 168:2011-2019 (2002). EAE is induced in female SJL/J mice (8-10 wk of age) immunized by s.c. injection at two sites at the tail base on day 0, and boosted on day 7 in the flanks, with 400 μg guinea pig MBP (Sigma, St. Louis, MO) emulsified in CFA containing 50 μg heat-inactivated Mycobacterium tuberculosis H37Ra (Sigma), in a volume of 50 μl/site. The disease develops with an acute phase characterized by loss of weight and progressive ascending clinical paralysis, followed by periods of remission with weight recovery, and subsequent relapses or chronic disease. Clinical symptoms are scored, e.g. no symptoms, flaccid tail, impairment of righting reflex or abnormal gait, severe hind limb weakness, complete hind limb paralysis, or paraplegia, moribund. Various concentrations of modified mRNA encoding G-CSF are administered. As a control recombinant human G-CSF (Amgen, Thousand Oaks, CA) is injected s.c. at 200 μg/kg/day. The excipient consists of 5% dextrose in sterile H₂O. The disease is followed histologically and immunohistochemically. TNF-α concentration in serum is measured, cytokine and chemokine levels and the T cell autoreactive response is assessed.

### Example 10

The effects of modified mRNA encoding G-CSF on autoimmune disease is tested using an MRL-^{lpr/lpr} mouse model, a genetic model of the human autoimmune disease systemic lupus erythematosus. Six-week-old female MRL-lpr/lpr mice are maintained under specific pathogen-free conditions. Modified mRNA encoding G-CSF or recombinant human G-CSF (Amgen, Thousand Oaks, CA) is injected s.c. every 6 wk for five consecutive days, starting at 9 wk of age. Mice at this age have no albuminuria, they have detectable anti-nucleosome but no anti-dsDNA antibodies, which is typical of the onset of the autoantibody response in young lupus mice. As controls, two doses of recombinant G-CSF are used: 10 μg/kg and 200 μg/kg, while the other control group receives the carrier solution (5% dextrose in sterile H₂0), according to Zavala, F et al. J Immunol 163: 5125 (1999). Renal disease is evaluated by the development of albuminuria and histological modifications of the kidney. Albuminuria was measured colorimetrically using commercially available sticks, with albumin concentrations over 1 mg/ml considered positive. Kidney histology is performed on mice killed at 20 wk of age. The kidneys are removed, fixed in 3.6% paraformaldehyde in PBS, and included in paraffin. Sections of 5 μm are stained with hematoxylin-eosin and analyzed for glomerulonephritis. Evaluation of the Ig deposits in the glomeruli is performed on frozen kidneys frozen. Spleen and bone marrow cells are analyzed by FACS. Cytokine production by splenocytes and cytokine concentrations in serum are measured.

### Example 11

The nucleic acid sequence for ACTH1-17 resulting in human alpha-melanocyte
stimulating hormone (α-MSH) is set forth in SEQ ID NO. 2:
(SEQ ID No. 2)
aagcttttggaccctcgtacagaagctaatacgactcactatagggaaataagagagaaaagaagagtaagaagaaatataagagccacc
atgcctagatcatgttgttcacgatcgggagcgttgttgctggcactcttgctccaagcctcgatggaagtgcggggatggtgccttgaga
gctcccagtgccaggacctcacaacggagtcgaaccttctggaatgcatccgcgcgtgtaaaccgagggaaggtaaaaggtcctacagcat
ggagcacttcagatgggggaagcccgtcgggaagaagcggtgaagcgctgccttctgcggggcttgccttctggccatgcccttcttctct
cccttgcacctgtacctcttggtctttgaataaagcctgagtaggaaggggccgctcgagcatgcatctagag
The nucleic acid sequence for α-MSH mRNA is set forth in SEQ ID NO. 19:
(SEQ ID No. 19)
aagcuuuuggacccucguacagaagcuaauacgacucacuauagggaaauaagagagaaaagaagaguaagaagaaauauaag
agccaccaugccuagaucauguuguucacgaucgggagcguuguugcuggcacucuugcuccaagccucgauggaagugcgg
ggauggugccuugagagcucccagugccaggaccucacaacggagucgaaccuucuggaaugcaucegcgcguguaaaccga
gggaagguaaaagguccuacagcauggagcacuucagaugggggaagcccgucgggaagaagcggugaagcgcugccuucug
cggggcuugccuucuggccaugcccuucuucucucccuugcaccuguaccucuugguuuugaaaaagccugaguaggaag
gcggccgcucgagcaugcaucuagag
The nucleic acid sequence for an exemplary α-MSH modified mRNA (mmRNA) is
set forth in SEQ ID NO. 20:
(SEQ ID No. 20)
aag5meCψψψψgga5meC5meC5meCψ5meCgψa5meCagaag5meCψaaψa5meCga5meCψ5meCa5
meCψaψagggaaaψaagagagaaaagaagagψaagaagaaaψaψaagag5meC5meCa5meC5meCaψg5meC5
meCψagaψ5meCaψgψψgψψ5meCa5meCgaψ5meCgggag5meCgvψgψψg5meCψgg5meCa5meC
ψ5meCψψg5meCψ5meC5meCaag5meC5meCψ5meCgaψggaagψg5meCggggaψggψg5meC5me
Cψψgagag5meCψ5meC5meC5meCagψg5meC5meCagga5meC5meCψ5meCa5meCaa5meCgga
gψ5meCgaa5meC5meCψψ5meCψggaaψg5meCaψ5meC5meCg5meCg5meCgψgψaaa5meC5me
Cgagggaaggψaaaaggψ5meC5meCψa5meCag5meCaψggag5meCa5meCψψ5meCagaψgggggaag5
meC5meC5meCgψ5meCgggaagaag5meCggψgaag5meCg5meCψg5meC5meCψψ5meCψg5meC
gggg5meCψψg5meC5meCψψ5meCψgg5meC5meCaψg5meC5meC5meCψψ5meCψψ5meCψ5m
eCψ5meC5meC5meCψψg5meCa5meC5meCψgψa5meC5meCψ5meCψψggψ5meCψψψgaaψaaa
g5meC5meCψgagψaggaagg5meCgg5meC5meCg5meCψ5meCgag5meCaψg5meCaψ5meCψaga
g

The amino acid sequence of ACTH1-17 is set forth in SEQ ID No. 9: SYSMEHFRWGKPVGKKR. The C-terminal end contains amino acids necessary for amidation of αMSH (GKK). Melanocyte-stimulating hormone (αMSH) is a 13 amino acid peptide derived from pro-opiomelanocortin (POMC), a pro-hormone that is post-translationally processed. POMC contains a signal peptide (amino acid residues 1-26) and a sorting peptide (amino acid residues 27-52) that direct POMC to the lumen of the endoplasmic reticulum (ER) where it is processed by hormone convertases (PC1 and PC2). Native αMSH is produced via cleavage of ACTH1-17 to ACTH1-13, followed by amidation of the carboxyl terminus (the signal for amidation is located at amino acids 14-16 of ACTH (Gly, Lys, Lys)) and acetylation of the amino terminus. The release of αMSH from the cell occurs in response to certain stimuli with some constitutive secretion activity. Yin P et al. Gene Therapy 10:348-355 (2003). FIG. 1b shows an Enzyme-linked immunosorbent assay (ELISA) for Human alpha-Melanocyte Stimulating Hormone (α-MSH) of in vitro transfected Human Melanocyte cells. Melanocytes were grown in Medium 254 with supplement MHGS-2 from Invitrogen until they reached a confluence of 50-70%. Cells were transfected with 0, 50, 100, 250, 500, or 1000 ng mmRNA complexed with RNAiMAX from Invitrogen. The RNA:RNAiMAX complex was formed by first incubating the RNA with Supplement-free Medium 254 media in a 5× volumetric dilution for 10 minutes at room temperature. In a second vial, RNAiMAX reagent was incubated with Supplement-free Medium 254 Media in a 10× volumetric dilution for 10 minutes at room temperature. The RNA vial was then mixed with the RNAiMAX vial and incubated for 20-30 at room temperature before being added to the cells in a drop-wise fashion. Secreted α-MSH concentration in the culture medium was measured at 18, 24, and 48 hours post-transfection for each of the 6 concentrations. Secretion of α-MSH from transfected human melanocytes was quantified using an ELISA kit from Phoenix Pharmaceuticals following the manufacturers recommended instructions. α-MSH shows a dose response, with 1000 ng mmRNA showing the best response. Also, production peaked at 48 hours after transfection. These data show that α-MSH mmRNA (SEQ ID NO: 2) is capable of being translated in Human Melanocytes cells and that α-MSH is transported out of the cells and released into the extracellular environment.

Mice (DBA/2 males approx. 8 weeks old (20 total mice), group housed) are topically treated (Dosing regimen: 3×/day for 3 days) with αMSH mmRNA (SEQ ID NO. 2) at 50 μg/dose to stimulate melanin production. Group Size: N=5 for each treatment group.

### Experimental Design

| Study compound | Control: | | | |
|---|---|---|---|---|
| mmRNA | Unmodified | Control | Control | Total |
| α-MSH | mRNA α-MSH | Scramble | Vehicle | Mice |
| Shave and | Shave and | Shave and | Shave and | 20 |
| topical | topical | topical | topical | |
| dosing, n = 5 | dosing, n = 5 | dosing, n = 5 | dosing, n = 5 | |

### Treatment Groups:

mmRNA α-MSH
Unmodified mRNA α-MSH
Control: non-specific RNA (total yeast RNA)
Control: Vehicle

### Diet:

Regular chow diet: Pico Rodent Chow (5053).

### Assay:

A patch of skin is shaved in the mid lumbar region and the compounds are applied to the skin. For 10 days, every 12 hours, the shaved patch is photographed (20 total photographs). The beginning and ending body weights are measured.

### Example 12

The nucleic acid sequence of mouse proinsulin is set forth in SEQ ID NO. 3:
(SEQ ID No. 3)
agcttttggaccctcgtacagaagctaatacgactcactatagggaaataagagagaaaagaagagtaagaagaaatataagagccaccat
ggcgctctggtgaggtttctgcctttgttggccttgctgttcctctgggagtcccaccccacacaggcgtttgtgaagcagcacttgtgcg
ggtcgcatctggtggaggcactttatctcgtctgcggcgagcgaggattcttctacaccccgatgagcagacgcgaagtggaagatccgca
agtcgcgcagctcgaacttgggggtggtcccggagccggagacttgcaaactctcgctctcgaggtagcgcagcagaaacggggtatcgta
gaccagtgttgcacgtcgatctgttcactttatcaacttgagaactactgtaattgaagcgctgccttctgcggggcttgccttctggcca
tgcccttcttctctcccttgcacctgtacctcttggtctttgaataaagcctgagtaggaaggggccgctcgagcatgcatctagagggcc
caattcgccctattcg
The nucleic acid sequence of human proinsulin is set forth in SEQ ID NO. 4:
(SEQ ID No. 4)
agcttttggaccctcgtacagaagctaatacgactcactatagggaaataagagagaaaagaagagtaagaagaaatataagagccaccat
ggctctctggatgcggcttcttccactgctcgccctcttggcgttgtggggacccgaccctgcagcagcgtttgtgaatcagcacctctgc
gggtcccatctggtcgaagccctttaccttgtgtgtggcgagcgcgggttcttctacacgcccaagacaaggcgcgaggcggaggatctcc
aagtagggcaggtggaattgggagggggaccgggagccggttcactccagcccctggcgttggaggggtcgctgcagaaaagaggtattg
tcgaacagtgttgcactagcatctgctcgctgtatcaacttgagaactattgtaactgaagcgctgccttctgcggggcttgccttctggc
catgcccttcttctctcccttgcacctgtacctcttggtctttgaataaagcctgagtaggaaggcggccgctcgagcatgcatctagagg
gcccaattcgccctattcgaag
The nucleic acid sequence of mouse proinsulin mRNA is set forth in SEQ ID NO. 21:
(SEQ ID No. 21)
agcuuuuggacccucguacagaagcuaauacgacucacuauagggaaauaagagagaaaagaagaguaagaagaaauauaaga
gccaccauggcgcucuggugagguuucugccuuuguuggccuugcuguuccucugggagucccaccccacacaggcguuug
ugaagcagcacuugugcgggucgcaucugguggaggcacuuuaucucgucugcggcgagcgaggauucuucuacaccccgau
gagcagacgcgaaguggaagauccgcaagucgcgcagcucgaacuuggggguggucccggagccggagacuugcaaacucuc
gcucucgagguagcgcagcagaaacgggguaucguagaccaguguugcacgucgaucuguucacuuuaucaacuugagaacu
cuuugaauaaagccugaguaggaaggggccgcucgagcaugcaucuagagggcccaauucgcccuauucg
The nucleic acid sequence of mouse proinsulin exemplary modified mRNA
(mmRNA) is set forth in SEQ ID NO. 22:
(SEQ ID No. 22)
ag5meCψψψψgga5meC5meC5meCψ5meCgψa5meCagaag5meCψaaψa5meCga5meCψ5meCa5
meCψaψagggaaaψaagagagaaaagaagagψaagaagaaaψaψaagag5meC5meCa5meC5meCaψgg5meC
g5meCψ5meCψggψgaggψψψ5meCψg5meC5meCψψψgψψgg5meC5meCψψg5meCψgψψ5meC
5meCψ5meCψgggagψ5meC5meC5meCa5meC5meC5meC5meCa5meCa5meCagg5meCgψψψg
ψgaag5meCag5meCa5meCψψgψg5meCgggψ5meCg5meCaψ5meCψggψggagg5meCa5meCψψψ
aψ5meCψ5meCgψ5meCψg5meCgg5meCgag5meCgaggaψψ5meCψψ5meCψa5meCa5meC5me
C5meC5meCgaψgag5meCaga5meCg5meCgaagψggaagaψ5meC5meCg5meCaagψ5meCg5meCg
5meCag5meCψ5meCgaa5meCψψgggggψggψ5meC5meC5meCggag5meC5meCggaga5meCψψg
5meCaaa5meCψ5meCψ5meCg5meCψ5meCψ5meCgaggψag5meCg5meCag5meCagaaa5meCgg
ggψaψ5meCgψaga5meC5meCagψgψψg5meCa5meCgψ5meCgaψ5meCψgψψ5meCa5meCψψψa
ψ5meCaa5meCψψgagaa5meCψa5meCψgψaaψψgaag5meCg5meCψg5meC5meCψψ5meCψg5m
eCgggg5meCψψg5meC5meCψψ5meCψgg5meC5meCaψg5meC5meC5meCψψ5meCψψ5meCψ
5meCψ5meC5meC5meCψψg5meCa5meC5meCψgψa5meC5meCψ5meCψψggψ5meCψψψgaaψ
aaag5meC5meCψgagψaggaagg5meCgg5meC5meCg5meCψ5meCgag5meCaψg5meCaψ5meCψ
agaggg5meC5meC5meCaaψψ5meCg5meC5meC5meCψaψψ5meCg
The nucleic acid sequence of human proinsulin mRNA is set forth in SEQ ID NO. 23:
(SEQ ID No. 23)
agcuuuuggacccucguacagaagcuaauacgacucacuauagggaaauaagagagaaaagaagaguaagaagaaauauaaga
gccaccauggcucucuggaugcggcuucuuccacugcucgcccucuuggguuguggggacccgacccugcagcagcguuug
ugaaucagcaccucugcgggucccaucuggucgaagcccuuuaccuuguguguggcgagcgcggguucuucuacacgcccaa
gacaaggcgcgaggcggaggaucuccaaguagggcagguggaauugggagggggaccgggagccgguucacuccagccccug
gcguuggaggggucgcugcagaaaagagguauugucgaacaguguugcacuagcaucugcucgcuguaucaacuugagaacu
auuguaacugaagcgcugcuucugcggggcuugccuucuggccaugcccuucuucucucccuugcaccuguaccucuuggu
cuuugaauaaagccugaguaggaaggggccgcucgagcaugcaucuagagggcccaauucgcccuauucgaag
The nucleic acid sequence of an exemplary human proinsulin modified mRNA
(mmRNA) is set forth in SEQ ID NO. 24:
(SEQ ID No. 24)
ag5meCψψψψgga5meC5meC5meCψ5meCgψa5meCagaag5meCψaaψa5meCga5meCψ5meCa5
meCψaψagggaaaψaagagagaaaagaagagψaagaagaaaψaψaagag5meC5meCa5meC5meCaψgg5meC
ψ5meCψ5meCψggaψg5meCgg5meCψψ5meCψψ5meC5meCa5meCψg5meCψ5meCg5meC5me
C5meCψ5meCψψgg5meCgψψgψgggga5meC5meC5meCga5meC5meC5meCψg5meCag5meCa
g5meCgψψψgψgaaψ5meCag5meCa5meC5meCψ5meCψg5meCgggψ5meC5meC5meCaψ5meC
ψggψ5meCgaag5meC5meC5meCψψψa5meC5meCψψgψgψgψgg5meCgag5meCg5meCgggψψ5
meCψψ5meCψa5meCa5meCg5meC5meC5meCaaga5meCaagg5meCg5meCgagg5meCggaggaψ
5meCψ5meC5meCaagψaggg5meCaggψggaaψψgggaggggga5meC5meCgggag5meC5meCggψψ5
meCa5meCψ5meC5meCag5meC5meC5meC5meCψgg5meCgψψggaggggψ5meCg5meCψg5me
Cagaaaagaggψaψψgψ5meCgaa5meCagψgψψg5meCa5meCψag5meCaψ5meCψg5meCψ5meCg5
meCψgψaψ5meCaa5meCψψgagaa5meCψaψψgψaa5meCψgaag5meCg5meCψg5meC5meCψψ5
meCψg5meCgggg5meCψψg5meC5meCψψ5meCψgg5meC5meCaψg5meC5meC5meCψψ5meC
ψψ5meCψ5meCψ5meC5meC5meCψψg5meCa5meC5meCψgψa5meC5meCψ5meCψψggψ5me
Cψψψgaaψaaag5meC5meCψgagψaggaagg5meCgg5meC5meCg5meCψ5meCgag5meCaψg5me
Caψ5meCψagaggg5meC5meC5meCaaψψ5meCg5meC5meC5meCψaψψ5meCgaag
The proinsulin contains a signal peptide that must be cleaved for the protein to be
secreted. The amino acid sequence of mouse insulin is set forth in SEQ ID NO: 15:
(SEQ ID NO: 15)
MALWMRFLPLLALLFLWESHPTQAFVKQHLCGSHLVEALYLVCGERGFFYTPMSRREV
EDPQVAQLELGGGPGAGDLQTLALEVAQQKRGIVDQCCTSICSLYQLENYCN
The amino acid sequence of human insulin is set forth in SEQ ID NO: 16:
(SEQ ID NO: 16)
MALWMRLLPLLALLALWGPDPAAAFVNQHLCGSHLVEALYLVCGERGFFYTPKTRRE
AEDLQVGQVELGGGPGAGSLQPLALEGSLQKRGIVEQCCTSICSLYQLENYCN

FIG. 1c shows an Enzyme-linked immunosorbent assay (ELISA) for Mouse Insulin of in vitro transfected Human Keratinocyte cells. Keratinocytes were grown in EpiLife medium with Supplement S7 from Invitrogen until they reached a confluence of 50-70%. Cells were transfected with 0, 100, 250, 500, 1000, and 1500 ng mmRNA complexed with RNAiMAX from Invitrogen. The RNA:RNAiMAX complex was formed by first incubating the RNA with Supplement-free EpiLife media in a 5× volumetric dilution for 10 minutes at room temperature. In a second vial, RNAiMAX reagent was incubated with Supplement-free EpiLife Media in a 10× volumetric dilution for 10 minutes at room temperature. The RNA vial was then mixed with the RNAiMAX vial and incubated for 20-30 at room temperature before being added to the cells in a drop-wise fashion. Secreted mu-Insulin concentration in the culture medium was measured at 6, 12, 24, and 36 hours post-transfection for each of the 6 concentrations. Secretion of Mouse Insulin from transfected human keratinocytes was quantified using an ELISA kit from Millipore following the manufacturers recommended instructions. Mouse Insulin shows a dose response, with 1500 ng mmRNA showing the best response. Also, production peaked between 24 and 36 hours after transfection. These data show that mu-Insulin mmRNA (SEQ ID NO: 3) is capable of being translated in Human Keratinocyte cells and that mu-Insulin is transported out of the cells and released into the extracellular environment.

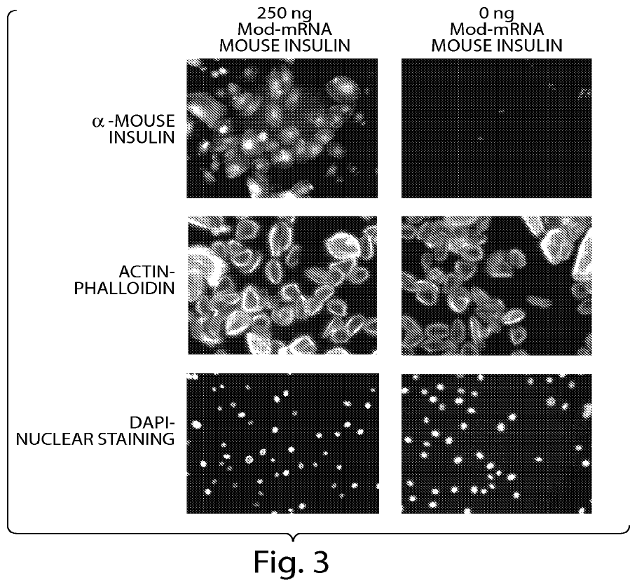

FIG. 3 shows human keratinocytes transfected with 0 or 250 ng of Mouse Insulin. Cells were grown in EpiLife Media with Supplement S7 from Invitrogen according to standard protocols in 24-well collagen-coated plates. Cells were fixed with 4% paraformaldehyde in PBS and permeabilized with 0.1% Triton X-100 in PBS for 5-10 minutes at room temperature. Cells were then washed 3× with room temperature PBS. Insulin protein staining was performed using Insulin mouse monoclonal antibody D6C4 (ab8304, abcam, Cambridge, MA) and goat polyclonal secondary antibody to mouse IgG conjugated to DyLight® 594 (ab97017, abcam) according to the manufacturer's recommended dilutions. Nuclear DNA staining was performed with DAPI dye from Invitrogen. The cellular actin cytoskeleton was stained using AlexaFluor488-conjugated phalloidin from Invitrogen. Insulin protein is translated and localized to the cytoplasm upon Insulin mmRNA transfection. The picture was taken 18 hours after transfection.

Mice (Streptozotocin-induced diabetic C57BL/6NTac mice, 10-12 weeks old, group housed, 96 total mice) are IV injected with mouse proinsulin or human proinsulin mmRNA (SEQ ID NOs: 3 and 4) to analyze glucohomeostasis. Group Size: N=8 for each treatment group.

### Experimental Design

| Treatment | Day | Dosing | |
|---|---|---|---|
| Acute study with recovery | | | |
| mmRNA hProinsulin | 1, 8 | 50 | μg |
| Dose 1 mmRNA mProinsulin | 1, 8 | 0.5 | μg |
| Dose 2 mmRNA mProinsulin | 1, 8 | 5 | μg |
| Dose 3 mmRNA mProinsulin | 1, 8 | 50 | μg |
| Dose 4 mmRNA mProinsulin | 1, 8 | 100 | μg |
| Control: unmodified mRNA mProinsulin | 1, 8 | 50 | μg |
| Control: scramble (yeast total mRNA) | 1, 8 | 50 | μg |
| Control: vehicle only | 1, 8 | 50 | μg |
| Sub-chronic study | | | |
| Dose 1 mmRNA mProinsulin | 1-5 | 50 | μg |
| Control: unmodified mRNA mProinsulin | 1-5 | 50 | μg |
| Control: non-specific RNA (yeast total RNA) | 1-5 | 50 | μg |
| Control: vehicle only | 1-5 | 50 | μg |

### Dosing Regimen:

Acute: IV in tail vein once, testing conducted 6 hours later

Sub-chronic: IV in tail vein once daily for 5 days, testing conducted 6 hours after final injection

### Diet:

Mice were given a high fat diet (HFD) (Research Diets D12492)

### Assays:

### Triglycerides

Whole blood is collected via the retro-orbital sinus and triglycerides are assessed on a hand held meter (CardioChek, PTS Inc. Indianapolis, IN) with test strips specific for triglyceride measurements, using approximately 20 μl of blood. Samples are tested in mice under three different conditions: fasted and freely fed on a regular diet, and fasted after 8 weeks of a high fat diet.

### Oral Glucose Tolerance Test Procedure

Mice are tested before a high fat diet challenge and 8 weeks after a high fat diet challenge. Mice are fasted for 16 hours and transferred to a procedure room midway through the light phase of the light/dark cycle. Blood is obtained from a tail cut and blood glucose levels are measured by a glucometer (One-touch Ultra II) and additional blood is processed for plasma that is later used to determine the fasting insulin levels. The mice receive 2 g/kg body weight of glucose by oral gavage. 15, 30, 60, and 120 minutes after the administration of glucose, blood is collected to measure the glucose concentration and to prepare plasma samples for measuring insulin levels. All of the plasma samples are frozen after collection and assayed simultaneously by electrochemiluminscence (MA2400 Mouse/Rat insulin kit K152BZC, MesoScale Discovery) according to the manufacturer's recommendations.

1. A pharmaceutical formulation comprising:
i) an effective amount of a synthetic messenger ribonucleic acid (mRNA) encoding a granulocyte colony-stimulating factor (G-CSF) polypeptide; and
ii) a pharmaceutically acceptable carrier,
wherein i) the mRNA comprises pseudouridine, 5′methyl-cytidine or a combination thereof, or ii) wherein the mRNA does not comprise a substantial amount of a nucleotide or nucleotides selected from the group consisting of uridine, cytidine, and a combination of uridine and cytidine, and wherein the formulation is suitable for repeated intravenous administration to a mammalian subject in need thereof.

2. The pharmaceutical formulation of claim 1, wherein the G-CSF polypeptide comprises:
a) the amino acid sequence of SEQ ID NOs: 13 or 14;
b) an amino acid sequence at least 95% identical to SEQ ID NOs: 13 or 14;
c) a fragment of SEQ ID NOs: 13 or 14;
d) the amino acid sequence encoded by the nucleic acid of SEQ ID NO: 2 or SEQ ID NO: 17; or
e) the amino acid sequence encoded by a nucleic acid at least 95% identical to SEQ ID NO: 2 or SEQ ID NO: 7

3. The pharmaceutical formulation of claim 1, further comprising a lipid-based transfection reagent.

4. The pharmaceutical formulation of claim 1, wherein the mRNA lacks at least one destabilizing element.

5. A kit comprising the pharmaceutical formulation of claim 1 and AMD3100 (1,1′-[1,4-phenylene-bis(methylene)]-bis-1,4,8,11-tetraazacyclotetradecane), in one or more containers, and instructions for use thereof.

6. A pharmaceutical formulation consisting essentially of:
i) an effective amount of a synthetic messenger ribonucleic acid (mRNA) encoding a granulocyte colony-stimulating factor (G-CSF) polypeptide;
ii) a cell penetration agent; and
iii) a pharmaceutically acceptable carrier,
wherein i) the mRNA comprises pseudouridine, 5′methyl-cytidine or a combination thereof, or ii) wherein the mRNA does not comprise a substantial amount of a nucleotide or nucleotides selected from the group consisting of uridine, cytidine, and a combination of uridine and cytidine, and wherein the formulation is suitable for repeated intravenous administration to a mammalian subject in need thereof.

7. A method to obtain progenitor and/or stem cells from a mammalian subject, comprising the steps of: (a) administering to the subject a composition comprising a synthetic messenger ribonucleic acid (mRNA) encoding a granulocyte colony-stimulating factor (G-CSF) polypeptide in an amount effective to mobilize the progenitor and/or stem cells into the peripheral blood of the subject; followed by (b) harvesting the progenitor and/or stem cells.

8. The method of claim 7 wherein the mRNA or a pharmaceutically acceptable salt thereof is administered to the subject in the dosage range of about 1.0 μg/kg-100 mg/kg of body weight.

9. The method of claim 7, wherein the subject is human.

10. The method of claim 7, wherein the composition is administered to the subject by an intravenous route.

11. The method of claim 7, further comprising the step of administering an effective amount of AMD3100 (1,1′-[1,4-phenylene-bis(methylene)]-bis-1,4,8,11-tetraazacyclotetradecane) to the subject after administering the composition.

12. The method of claim 11, wherein the composition is administered at least twice but fewer than ten times prior to administering the AMD3100.

13. The method of claim 7, wherein the composition further comprises a synthetic messenger ribonucleic acid (mRNA) encoding i) a macrophage inflammatory protein (MIP) polypeptide or ii) an antibody that prevents EGF binding by EGFR.

14-18. (canceled)


SEQUENCE LISTING (24 sequences)

SEQ ID NO: 1 - DNA, 852 bp, Homo sapiens
  source 1..852: mol_type = genomic DNA
agcttttggaccctcgtacagaagctaatacgactcactatagggaaataagagagaaaagaagagtaagaagaaatataagagccaccatggccggtcccgcgacccaaagccccatgaaacttatggccctgcagttgctgctttggcactcggccctctggacagtccaagaagcgactcctctcggacctgcctcatcgttgccgcagtcattccttttgaagtgtctggagcaggtgcgaaagattcagggcgatggagccgcactccaagagaagctctgcgcgacatacaaactttgccatcccgaggagctcgtactgctcgggcacagcttggggattccctgggctcctctctcgtcctgtccgtcgcaggctttgcagttggcagggtgcctttcccagctccactccggtttgttcttgtatcagggactgctgcaagcccttgagggaatctcgccagaattgggcccgacgctggacacgttgcagctcgacgtggcggatttcgcaacaaccatctggcagcagatggaggaactggggatggcacccgcgctgcagcccacgcagggggcaatgccggcctttgcgtccgcgtttcagcgcagggcgggtggagtcctcgtagcgagccaccttcaatcatttttggaagtctcgtaccgggtgctgagacatcttgcgcagccgtgaagcgctgccttctgcggggcttgccttctggccatgcccttcttctctcccttgcacctgtacctcttggtctttgaataaagcctgagtaggaaggcggccgctcgagcatgcatctagagggcccaattcgccctattcgaagtcg

SEQ ID NO: 2 - DNA, 437 bp, Homo sapiens
  source 1..437: mol_type = genomic DNA
aagcttttggaccctcgtacagaagctaatacgactcactatagggaaataagagagaaaagaagagtaagaagaaatataagagccaccatgcctagatcatgttgttcacgatcgggagcgttgttgctggcactcttgctccaagcctcgatggaagtgcggggatggtgccttgagagctcccagtgccaggacctcacaacggagtcgaaccttctggaatgcatccgcgcgtgtaaaccgagggaaggtaaaaggtcctacagcatggagcacttcagatgggggaagcccgtcgggaagaagcggtgaagcgctgccttctgcggggcttgccttctggccatgcccttcttctctcccttgcacctgtacctcttggtctttgaataaagcctgagtaggaaggcggccgctcgagcatgcatctagag

SEQ ID NO: 3 - DNA, 563 bp, Mus sp.
  source 1..563: mol_type = genomic DNA
agcttttggaccctcgtacagaagctaatacgactcactatagggaaataagagagaaaagaagagtaagaagaaatataagagccaccatggcgctctggtgaggtttctgcctttgttggccttgctgttcctctgggagtcccaccccacacaggcgtttgtgaagcagcacttgtgcgggtcgcatctggtggaggcactttatctcgtctgcggcgagcgaggattcttctacaccccgatgagcagacgcgaagtggaagatccgcaagtcgcgcagctcgaacttgggggtggtcccggagccggagacttgcaaactctcgctctcgaggtagcgcagcagaaacggggtatcgtagaccagtgttgcacgtcgatctgttcactttatcaacttgagaactactgtaattgaagcgctgccttctgcggggcttgccttctggccatgcccttcttctctcccttgcacctgtacctcttggtctttgaataaagcctgagtaggaaggcggccgctcgagcatgcatctagagggcccaattcgccctattcg

SEQ ID NO: 4 - DNA, 567 bp, Homo sapiens
  source 1..567: mol_type = genomic DNA
agcttttggaccctcgtacagaagctaatacgactcactatagggaaataagagagaaaagaagagtaagaagaaatataagagccaccatggctctctggatgcggcttcttccactgctcgccctcttggcgttgtggggacccgaccctgcagcagcgtttgtgaatcagcacctctgcgggtcccatctggtcgaagccctttaccttgtgtgtggcgagcgcgggttcttctacacgcccaagacaaggcgcgaggcggaggatctccaagtagggcaggtggaattgggagggggaccgggagccggttcactccagcccctggcgttggaggggtcgctgcagaaaagaggtattgtcgaacagtgttgcactagcatctgctcgctgtatcaacttgagaactattgtaactgaagcgctgccttctgcggggcttgccttctggccatgcccttcttctctcccttgcacctgtacctcttggtctttgaataaagcctgagtaggaaggcggccgctcgagcatgcatctagagggcccaattcgccctattcgaag

SEQ ID NO: 5 - DNA, 3547 bp, synthetic construct
  misc_feature 1..3547: note = Description of Artificial Sequence: Synthetic polynucleotide
  source 1..3547: mol_type = other DNA
accaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattatggggtgtcgcccttttgacgcgacttcgaatagggcgaattgggccctctagatgcatgctcgagcggccgccttcctactcaggctttattcaaagaccaagaggtacaggtgcaagggagagaagaagggcatggccagaaggcaagccccgcagaaggcagcgcttcacggctgcgcaagatgtctcagcacccggtacgagacttccaaaaatgattgaaggtggctcgctacgaggactccacccgccctgcgctgaaacgcggacgcaaaggccggcattgccccctgcgtgggctgcagcgcgggtgccatccccagttcctccatctgctgccagatggttgttgcgaaatccgccacgtcgagctgcaacgtgtccagcgtcgggcccaattctggcgagattccctcaagggcttgcagcagtccctgatacaagaacaaaccggagtggagctgggaaaggcaccctgccaactgcaaagcctgcgacggacaggacgagagaggagcccagggaatccccaagctgtgcccgagcagtacgagctcctcgggatggcaaagtttgtatgtcgcgcagagcttctcttggagtgcggctccatcgccctgaatctttcgcacctgctccagacacttcaaaaggaatgactgcggcaacgatgaggcaggtccgagaggagtcgcttcttggactgtccagagggccgagtgccaaagcagcaactgcagggccataagtttcatggggctttgggtcgcgggaccggccatggtggctcttatatttcttcttactcttcttttctctcttatttccctatagtgagtcgtattagcttctgtacgagggtccaaaagctttcagcgaagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgctttt

SEQ ID NO: 6 - DNA, 3262 bp, synthetic construct
  misc_feature 1..3262: note = Description of Artificial Sequence: Synthetic polynucleotide
  source 1..3262: mol_type = other DNA
accaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattatggggtgtcgcccttcgctgaaagcttttggaccctcgtacagaagctaatacgactcactatagggaaataagagagaaaagaagagtaagaagaaatataagagccaccatggctctctggatgcggcttcttccactgctcgccctcttggcgttgtggggacccgaccctgcagcagcgtttgtgaatcagcacctctgcgggtcccatctggtcgaagccctttaccttgtgtgtggcgagcgcgggttcttctacacgcccaagacaaggcgcgaggcggaggatctccaagtagggcaggtggaattgggagggggaccgggagccggttcactccagcccctggcgttggaggggtcgctgcagaaaagaggtattgtcgaacagtgttgcactagcatctgctcgctgtatcaacttgagaactattgtaactgaagcgctgccttctgcggggcttgccttctggccatgcccttcttctctcccttgcacctgtacctcttggtctttgaataaagcctgagtaggaaggcggccgctcgagcatgcatctagagggcccaattcgccctattcgaagcgtcaaaagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgctttt

SEQ ID NO: 7 - DNA, 3259 bp, synthetic construct
  misc_feature 1..3259: note = Description of Artificial Sequence: Synthetic polynucleotide
  source 1..3259: mol_type = other DNA
accaatgcttaatcagtgaggcacctatctcagcgatctgtctatttcgttcatccatagttgcctgactccccgtcgtgtagataactacgatacgggagggcttaccatctggccccagcgctgcgatgataccgcgagaaccacgctcaccggctccggatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccatcgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattatggggtgtcgcccttcgctgaaagcttttggaccctcgtacagaagctaatacgactcactatagggaaataagagagaaaagaagagtaagaagaaatataagagccaccatggcgctctggatgaggtttctgcctttgttggccttgctgttcctctgggagtcccaccccacacaggcgtttgtgaagcagcacttgtgcgggtcgcatctggtggaggcactttatctcgtctgcggcgagcgaggattcttctacaccccgatgagcagacgcgaagtggaagatccgcaagtcgcgcagctcgaacttgggggtggtcccggagccggagacttgcaaactctcgctctcgaggtagcgcagcagaaacggggtatcgtagaccagtgttgcacgtcgatctgttcactttatcaacttgagaactactgtaattgaagcgctgccttctgcggggcttgccttctggccatgcccttcttctctcccttgcacctgtacctcttggtctttgaataaagcctgagtaggaaggcggccgctcgagcatgcatctagagggcccaattcgccctattcgcgtcaaaagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgctttt

SEQ ID NO: 8 - DNA, 3081 bp, synthetic construct
  misc_feature 1..3081: note = Description of Artificial Sequence: Synthetic polynucleotide
  source 1..3081: mol_type = other DNA
tagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttcgctgaataagcttttggaccctcgtacagaagctaatacgactcactatagggaaataagagagaaaagaagagtaagaagaaatataagagccaccatgcctagatcatgttgttcacgatcgggagcgttgttgctggcactcttgctccaagcctcgatggaagtgcggggatggtgccttgagagctcccagtgccaggacctcacaacggagtcgaaccttctggaatgcatccgcgcgtgtaaaccgagggaaggtaaaaggtcctacagcatggagcacttcagatgggggaagcccgtcgggaagaagcggtgaagcgctgccttctgcggggcttgccttctggccatgcccttcttctctcccttgcacctgtacctcttggtctttgaataaagcctgagtaggaaggcggccgctcgagcatgcatctagagcgtcaaagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttt

SEQ ID NO: 9 - protein, 17 aa, Homo sapiens
SYSMEHFRWGKPVGKKR

SEQ ID NO: 10 - protein, 13 aa, Homo sapiens
SYSMEHFRWGKPV

SEQ ID NO: 11 - protein, 22 aa, Homo sapiens
AEKKDEGPYRMEHFRWGSPPKD

SEQ ID NO: 12 - protein, 12 aa, Homo sapiens
YVMGHFRWDRFG

SEQ ID NO: 13 - protein, 207 aa, Homo sapiens
MAGPATQSPMKLMALQLLLWHSALWTVQEATPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 14 - protein, 175 aa, Homo sapiens
MTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 15 - protein, 110 aa, Mus sp.
MALWMRFLPLLALLFLWESHPTQAFVKQHLCGSHLVEALYLVCGERGFFYTPMSRREVEDPQVAQLELGGGPGAGDLQTLALEVAQQKRGIVDQCCTSICSLYQLENYCN

SEQ ID NO: 16 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 17 - RNA, 852 nt, Homo sapiens
  source 1..852: mol_type = mRNA
agcttttggaccctcgtacagaagctaatacgactcactatagggaaataagagagaaaagaagagtaagaagaaatataagagccaccatggccggtcccgcgacccaaagccccatgaaacttatggccctgcagttgctgctttggcactcggccctctggacagtccaagaagcgactcctctcggacctgcctcatcgttgccgcagtcattccttttgaagtgtctggagcaggtgcgaaagattcagggcgatggagccgcactccaagagaagctctgcgcgacatacaaactttgccatcccgaggagctcgtactgctcgggcacagcttggggattccctgggctcctctctcgtcctgtccgtcgcaggctttgcagttggcagggtgcctttcccagctccactccggtttgttcttgtatcagggactgctgcaagcccttgagggaatctcgccagaattgggcccgacgctggacacgttgcagctcgacgtggcggatttcgcaacaaccatctggcagcagatggaggaactggggatggcacccgcgctgcagcccacgcagggggcaatgccggcctttgcgtccgcgtttcagcgcagggcgggtggagtcctcgtagcgagccaccttcaatcatttttggaagtctcgtaccgggtgctgagacatcttgcgcagccgtgaagcgctgccttctgcggggcttgccttctggccatgcccttcttctctcccttgcacctgtacctcttggtctttgaataaagcctgagtaggaaggcggccgctcgagcatgcatctagagggcccaattcgccctattcgaagtcg

SEQ ID NO: 18 - RNA, 852 nt, synthetic construct; identical to SEQ ID NO: 17
  misc_feature 1..852: note = Description of Artificial Sequence: Synthetic polynucleotide
  modified_base 3: mod_base = OTHER; note = 5-methyl-cytidine
  modified_base 4..7: mod_base = OTHER; note = Pseudouridine
  modified_base 11..13: mod_base = OTHER; note = 5-methyl-cytidine
  modified_base 14: mod_base = OTHER; note = Pseudouridine
  modified_base 15: mod_base = OTHER; note = 5-methyl-cytidine
  modified_base 17: mod_base = OTHER; note = Pseudouridine
  modified_base 19: mod_base = OTHER; note = 5-methyl-cytidine
  modified_base 25: mod_base = OTHER; note = 5-methyl-cytidine
  modified_base 26: mod_base = OTHER; note = Pseudouridine
  modified_base 29: mod_base = OTHER; note = Pseudouridine
  modified_base 31: mod_base = OTHER; note = 5-methyl-cytidine
  modified_base 34: mod_base = OTHER; note = 5-methyl-cytidine
  modified_base 35: mod_base = OTHER; note = Pseudouridine
  modified_base 36: mod_base = OTHER; note = 5-methyl-cytidine
  modified_base 38: mod_base = OTHER; note = 5-methyl-cytidine
  modified_base 39: mod_base = OTHER; note = Pseudouridine
  modified_base 41: mod_base = OTHER; note = Pseudouridine
  modified_base 49: mod_base = OTHER; note = Pseudouridine
  modified_base 67: mod_base = OTHER; note = Pseudouridine
  modified_base 77: mod_base = OTHER; note = Pseudouridine
  modified_base 79: mod_base = OTHER; note = Pseudouridine
  modified_base 85..86: mod_base = OTHER; note = 5-methyl-cytidine
  modified_base 88..89: mod_base = OTHER; note = 5-methyl-cytidine
  modified_base 91: mod_base = OTHER; note = Pseudouridine
  modified_base 94..95: mod_base = OTHER; note = 5-methyl-cytidine
  modified_base 98: mod_base = OTHER; note = Pseudouridine
  modified_base 99..101: mod_base = OTHER; note = 5-methyl-cytidine
  modified_base 103: mod_base = OTHER; note = 5-methyl-cytidine
  modified_base 106..108: mod_base = OTHER; note = 5-methyl-cytidine
  modified_base 113..116: mod_base = OTHER; note = 5-methyl-cytidine
  modified_base 118: mod_base = OTHER; note = Pseudouridine
  modified_base 123: mod_base = OTHER; note = 5-methyl-cytidine
  modified_base 124..125: mod_base = OTHER; note = Pseudouridine
  modified_base 127: mod_base = OTHER; note = Pseudouridine
  modified_base 130..132: mod_base = OTHER; note = 5-methyl-cytidine
  modified_base 133: mod_base = OTHER; note = Pseudouridine
  modified_base 135: mod_base = OTHER; note = 5-methyl-cytidine
  modified_base 138..139: mod_base = OTHER; note = Pseudouridine
  modified_base 141: mod_base = OTHER; note = 5-methyl-cytidine
  modified_base 142: mod_base = OTHER; note = Pseudouridine
  modified_base 144: mod_base = OTHER; note = 5-methyl-cytidine
  modified_base 145..147: mod_base = OTHER; note = Pseudouridine
  modified_base 150: mod_base = OTHER; note = 5-methyl-cytidine
  modified_base 152: mod_base = OTHER; note = 5-methyl-cytidine
  modified_base 153: mod_base = OTHER; note = Pseudouridine
  modified_base 154: mod_base = OTHER; note = 5-methyl-cytidine
  modified_base 157..159: mod_base = OTHER; note = 5-methyl-cytidine
  modified_base 160: mod_base = OTHER; note = Pseudouridine
  modified_base 161: mod_base = OTHER; note = 5-methyl-cytidine
  modified_base 162: mod_base = OTHER; note = Pseudouridine
  modified_base 166: mod_base = OTHER; note = 5-methyl-cytidine
  modified_base 169: mod_base = OTHER; note = Pseudouridine
  modified_base 170..171: mod_base = OTHER; note = 5-methyl-cytidine
  modified_base 178: mod_base = OTHER; note = 5-methyl-cytidine
  modified_base 181: mod_base = OTHER; note = 5-methyl-cytidine
  modified_base 182: mod_base = OTHER; note = Pseudouridine
  modified_base 183..184: mod_base = OTHER; note = 5-methyl-cytidine
  modified_base 185: mod_base = OTHER; note = Pseudouridine
  modified_base 186: mod_base = OTHER; note = 5-methyl-cytidine
  modified_base 187: mod_base = OTHER; note = Pseudouridine
  modified_base 188: mod_base = OTHER; note = 5-methyl-cytidine
  modified_base 192..193: mod_base = OTHER; note = 5-methyl-cytidine
  modified_base 194: mod_base = OTHER; note = Pseudouridine
  modified_base 196..197: mod_base = OTHER; note = 5-methyl-cytidine
  modified_base 198: mod_base = OTHER; note = Pseudouridine
  modified_base 199: mod_base = OTHER; note = 5-methyl-cytidine
  modified_base 201: mod_base = OTHER; note = Pseudouridine
  modified_base 202: mod_base = OTHER; note = 5-methyl-cytidine
  modified_base 204..205: mod_base = OTHER; note = Pseudouridine
  modified_base 207..208: mod_base = OTHER; note = 5-methyl-cytidine
  modified_base 210: mod_base = OTHER; note = 5-methyl-cytidine
  modified_base 213: mod_base = OTHER; note = Pseudouridine
  modified_base 214: mod_base = OTHER; note = 5-methyl-cytidine
  modified_base 216..217: mod_base = OTHER; note = Pseudouridine
  modified_base 218..219: mod_base = OTHER; note = 5-methyl-cytidine
  modified_base 220..223: mod_base = OTHER; note = Pseudouridine
  modified_base 228: mod_base = OTHER; note = Pseudouridine
  modified_base 230: mod_base = OTHER; note = Pseudouridine
  modified_base 231: mod_base = OTHER; note = 5-methyl-cytidine
  modified_base 232: mod_base = OTHER; note = Pseudouridine
  modified_base 237: mod_base = OTHER; note = 5-methyl-cytidine
  modified_base 241: mod_base = OTHER; note = Pseudouridine
  modified_base 243: mod_base = OTHER; note = 5-methyl-cytidine
  modified_base 250..251: mod_base = OTHER; note = Pseudouridine
  modified_base 252: mod_base = OTHER; note = 5-methyl-cytidine
  modified_base 257: mod_base = OTHER; note = 5-methyl-cytidine
  modified_base 260: mod_base = OTHER; note = Pseudouridine
  modified_base 265..266: mod_base = OTHER; note = 5-methyl-cytidine
  modified_base 268: mod_base = OTHER; note = 5-methyl-cytidine
  modified_base 270: mod_base = OTHER; note = 5-methyl-cytidine
  modified_base 271: mod_base = OTHER; note = Pseudouridine
  modified_base 272..273: mod_base = OTHER; note = 5-methyl-cytidine
  modified_base 282: mod_base = OTHER; note = 5-methyl-cytidine
  modified_base 283: mod_base = OTHER; note = Pseudouridine
  modified_base 284: mod_base = OTHER; note = 5-methyl-cytidine
  modified_base 285: mod_base = OTHER; note = Pseudouridine
  modified_base 287: mod_base = OTHER; note = 5-methyl-cytidine
  modified_base 289: mod_base = OTHER; note = 5-methyl-cytidine
  modified_base 292: mod_base = OTHER; note = 5-methyl-cytidine
  modified_base 294: mod_base = OTHER; note = Pseudouridine
  modified_base 296: mod_base = OTHER; note = 5-methyl-cytidine
  modified_base 300: mod_base = OTHER; note = 5-methyl-cytidine
  modified_base 301..303: mod_base = OTHER; note = Pseudouridine
  modified_base 305..306: mod_base = OTHER; note = 5-methyl-cytidine
  modified_base 308: mod_base = OTHER; note = Pseudouridine
  modified_base 309..311: mod_base = OTHER; note = 5-methyl-cytidine
  modified_base 318: mod_base = OTHER; note = 5-methyl-cytidine
  modified_base 319: mod_base = OTHER; note = Pseudouridine
  modified_base 320: mod_base = OTHER; note = 5-methyl-cytidine
  modified_base 322: mod_base = OTHER; note = Pseudouridine
  modified_base 324: mod_base = OTHER; note = 5-methyl-cytidine
  modified_base 325: mod_base = OTHER; note = Pseudouridine
  modified_base 327: mod_base = OTHER; note = 5-methyl-cytidine
  modified_base 328: mod_base = OTHER; note = Pseudouridine
  modified_base 329: mod_base = OTHER; note = 5-methyl-cytidine
  modified_base 333: mod_base = OTHER; note = 5-methyl-cytidine
  modified_base 335: mod_base = OTHER; note = 5-methyl-cytidine
  modified_base 338: mod_base = OTHER; note = 5-methyl-cytidine
  modified_base 339..340: mod_base = OTHER; note = Pseudouridine
  modified_base 346..347: mod_base = OTHER; note = Pseudouridine
  modified_base 348..350: mod_base = OTHER; note = 5-methyl-cytidine
  modified_base 351: mod_base = OTHER; note = Pseudouridine
  modified_base 355: mod_base = OTHER; note = 5-methyl-cytidine
  modified_base 356: mod_base = OTHER; note = Pseudouridine
  modified_base 357..358: mod_base = OTHER; note = 5-methyl-cytidine
  modified_base 359: mod_base = OTHER; note = Pseudouridine
  modified_base 360: mod_base = OTHER; note = 5-methyl-cytidine
  modified_base 361: mod_base = OTHER; note = Pseudouridine
  modified_base 362: mod_base = OTHER; note = 5-methyl-cytidine
  modified_base 363: mod_base = OTHER; note = Pseudouridine
  modified_base 364: mod_base = OTHER; note = 5-methyl-cytidine
  modified_base 366: mod_base = OTHER; note = Pseudouridine
  modified_base 367..368: mod_base = OTHER; note = 5-methyl-cytidine
  modified_base 369: mod_base = OTHER; note = Pseudouridine
  modified_base 371: mod_base = OTHER; note = Pseudouridine
  modified_base 372..373: mod_base = OTHER; note = 5-methyl-cytidine
  modified_base 375: mod_base = OTHER; note = Pseudouridine
  modified_base 376: mod_base = OTHER; note = 5-methyl-cytidine
  modified_base 378: mod_base = OTHER; note = 5-methyl-cytidine
  modified_base 382: mod_base = OTHER; note = 5-methyl-cytidine
  modified_base 383..385: mod_base = OTHER; note = Pseudouridine
  modified_base 387: mod_base = OTHER; note = 5-methyl-cytidine
  modified_base 390..391: mod_base = OTHER; note = Pseudouridine
  modified_base 394: mod_base = OTHER; note = 5-methyl-cytidine
  modified_base 399: mod_base = OTHER; note = Pseudouridine
  modified_base 401..402: mod_base = OTHER; note = 5-methyl-cytidine
  modified_base 403..405: mod_base = OTHER; note = Pseudouridine
  modified_base 406..408: mod_base = OTHER; note = 5-methyl-cytidine
  modified_base 411: mod_base = OTHER; note = 5-methyl-cytidine
  modified_base 412: mod_base = OTHER; note = Pseudouridine
  modified_base 413..414: mod_base = OTHER; note = 5-methyl-cytidine
  modified_base 416: mod_base = OTHER; note = 5-methyl-cytidine
  modified_base 417: mod_base = OTHER; note = Pseudouridine
  modified_base 418..419: mod_base = OTHER; note = 5-methyl-cytidine
  modified_base 422..424: mod_base = OTHER; note = Pseudouridine
  modified_base 426..427: mod_base = OTHER; note = Pseudouridine
  modified_base 428: mod_base = OTHER; note = 5-methyl-cytidine
  modified_base 429..430: mod_base = OTHER; note = Pseudouridine
  modified_base 432: mod_base = OTHER; note = Pseudouridine
  modified_base 434: mod_base = OTHER; note = Pseudouridine
  modified_base 435: mod_base = OTHER; note = 5-methyl-cytidine
  modified_base 441: mod_base = OTHER; note = 5-methyl-cytidine
  modified_base 442: mod_base = OTHER; note = Pseudouridine
  modified_base 444: mod_base = OTHER; note = 5-methyl-cytidine
  modified_base 445: mod_base = OTHER; note = Pseudouridine
  modified_base 447: mod_base = OTHER; note = 5-methyl-cytidine
  modified_base 451..453: mod_base = OTHER; note = 5-methyl-cytidine
  modified_base 454..455: mod_base = OTHER; note = Pseudouridine
  modified_base 463: mod_base = OTHER; note = Pseudouridine
  modified_base 464: mod_base = OTHER; note = 5-methyl-cytidine
  modified_base 465: mod_base = OTHER; note = Pseudouridine
  modified_base 466: mod_base = OTHER; note = 5-methyl-cytidine
  modified_base 468..469: mod_base = OTHER; note = 5-methyl-cytidine
  modified_base 474..475: mod_base = OTHER; note = Pseudouridine
  modified_base 479..481: mod_base = OTHER; note = 5-methyl-cytidine
  modified_base 484: mod_base = OTHER; note = 5-methyl-cytidine
  modified_base 486: mod_base = OTHER; note = 5-methyl-cytidine
  modified_base 487: mod_base = OTHER; note = Pseudouridine
  modified_base 491: mod_base = OTHER; note = 5-methyl-cytidine
  modified_base 493: mod_base = OTHER; note = 5-methyl-cytidine
  modified_base 495..496: mod_base = OTHER; note = Pseudouridine
  modified_base 498: mod_base = OTHER; note = 5-methyl-cytidine
  modified_base 501: mod_base = OTHER; note = 5-methyl-cytidine
  modified_base 502: mod_base = OTHER; note = Pseudouridine
  modified_base 503: mod_base = OTHER; note = 5-methyl-cytidine
  modified_base 506: mod_base = OTHER; note = 5-methyl-cytidine
  modified_base 508: mod_base = OTHER; note = Pseudouridine
  modified_base 511: mod_base = OTHER; note = 5-methyl-cytidine
  modified_base 515..517: mod_base = OTHER; note = Pseudouridine
  modified_base 518: mod_base = OTHER; note = 5-methyl-cytidine
  modified_base 520: mod_base = OTHER; note = 5-methyl-cytidine
  modified_base 523: mod_base = OTHER; note = 5-methyl-cytidine
  modified_base 526..527: mod_base = OTHER; note = 5-methyl-cytidine
  modified_base 529: mod_base = OTHER; note = Pseudouridine
  modified_base 530: mod_base = OTHER; note = 5-methyl-cytidine
  modified_base 531: mod_base = OTHER; note = Pseudouridine
  modified_base 534: mod_base = OTHER; note = 5-methyl-cytidine
  modified_base 537: mod_base = OTHER; note = 5-methyl-cytidine
  modified_base 541: mod_base = OTHER; note = Pseudouridine
  modified_base 549: mod_base = OTHER; note = 5-methyl-cytidine
  modified_base 550: mod_base = OTHER; note = Pseudouridine
  modified_base 556: mod_base = OTHER; note = Pseudouridine
  modified_base 559: mod_base = OTHER; note = 5-methyl-cytidine
  modified_base 561..563: mod_base = OTHER; note = 5-methyl-cytidine
  modified_base 565: mod_base = OTHER; note = 5-methyl-cytidine
  modified_base 567: mod_base = OTHER; note = 5-methyl-cytidine
  modified_base 568: mod_base = OTHER; note = Pseudouridine
  modified_base 570: mod_base = OTHER; note = 5-methyl-cytidine
  modified_base 573..575: mod_base = OTHER; note = 5-methyl-cytidine
  modified_base 577: mod_base = OTHER; note = 5-methyl-cytidine
  modified_base 579: mod_base = OTHER; note = 5-methyl-cytidine
  modified_base 586: mod_base = OTHER; note = 5-methyl-cytidine
  modified_base 589: mod_base = OTHER; note = Pseudouridine
  modified_base 591..592: mod_base = OTHER; note = 5-methyl-cytidine
  modified_base 595..596: mod_base = OTHER; note = 5-methyl-cytidine
  modified_base 597..599: mod_base = OTHER; note = Pseudouridine
  modified_base 601: mod_base = OTHER; note = 5-methyl-cytidine
  modified_base 603: mod_base = OTHER; note = Pseudouridine
  modified_base 604..605: mod_base = OTHER; note = 5-methyl-cytidine
  modified_base 607: mod_base = OTHER; note = 5-methyl-cytidine
  modified_base 609..611: mod_base = OTHER; note = Pseudouridine
  modified_base 612: mod_base = OTHER; note = 5-methyl-cytidine
  modified_base 615: mod_base = OTHER; note = 5-methyl-cytidine
  modified_base 617: mod_base = OTHER; note = 5-methyl-cytidine
  modified_base 622: mod_base = OTHER; note = 5-methyl-cytidine
  modified_base 626: mod_base = OTHER; note = Pseudouridine
  modified_base 631: mod_base = OTHER; note = Pseudouridine
  modified_base 632..633: mod_base = OTHER; note = 5-methyl-cytidine
  modified_base 634: mod_base = OTHER; note = Pseudouridine
  modified_base 635: mod_base = OTHER; note = 5-methyl-cytidine
  modified_base 637: mod_base = OTHER; note = Pseudouridine
  modified_base 640: mod_base = OTHER; note = 5-methyl-cytidine
  modified_base 644..645: mod_base = OTHER; note = 5-methyl-cytidine
  modified_base 647..648: mod_base = OTHER; note = 5-methyl-cytidine
  modified_base 649..650: mod_base = OTHER; note = Pseudouridine
  modified_base 651: mod_base = OTHER; note = 5-methyl-cytidine
  modified_base 654: mod_base = OTHER; note = Pseudouridine
  modified_base 655: mod_base = OTHER; note = 5-methyl-cytidine
  modified_base 657..661: mod_base = OTHER; note = Pseudouridine
  modified_base 667: mod_base = OTHER; note = Pseudouridine
  modified_base 668: mod_base = OTHER; note = 5-methyl-cytidine
  modified_base 669: mod_base = OTHER; note = Pseudouridine
  modified_base 670: mod_base = OTHER; note = 5-methyl-cytidine
  modified_base 672: mod_base = OTHER; note = Pseudouridine
  modified_base 674..675: mod_base = OTHER; note = 5-methyl-cytidine
  modified_base 679: mod_base = OTHER; note = Pseudouridine
  modified_base 681: mod_base = OTHER; note = 5-methyl-cytidine
  modified_base 682: mod_base = OTHER; note = Pseudouridine
  modified_base 687: mod_base = OTHER; note = 5-methyl-cytidine
  modified_base 689: mod_base = OTHER; note = Pseudouridine
  modified_base 690: mod_base = OTHER; note = 5-methyl-cytidine
  modified_base 691..692: mod_base = OTHER; note = Pseudouridine
  modified_base 694: mod_base = OTHER; note = 5-methyl-cytidine
  modified_base 696: mod_base = OTHER; note = 5-methyl-cytidine
  modified_base 699..700: mod_base = OTHER; note = 5-methyl-cytidine
  modified_base 702: mod_base = OTHER; note = Pseudouridine
  modified_base 707: mod_base = OTHER; note = 5-methyl-cytidine
  modified_base 709: mod_base = OTHER; note = 5-methyl-cytidine
  modified_base 710: mod_base = OTHER; note = Pseudouridine
  modified_base 712..713: mod_base = OTHER; note = 5-methyl-cytidine
  modified_base 714..715: mod_base = OTHER; note = Pseudouridine
  modified_base 716: mod_base = OTHER; note = 5-methyl-cytidine
  modified_base 717: mod_base = OTHER; note = Pseudouridine
  modified_base 719: mod_base = OTHER; note = 5-methyl-cytidine
  modified_base 724: mod_base = OTHER; note = 5-methyl-cytidine
  modified_base 725..726: mod_base = OTHER; note = Pseudouridine
  modified_base 728..729: mod_base = OTHER; note = 5-methyl-cytidine
  modified_base 730..731: mod_base = OTHER; note = Pseudouridine
  modified_base 732: mod_base = OTHER; note = 5-methyl-cytidine
  modified_base 733: mod_base = OTHER; note = Pseudouridine
  modified_base 736..737: mod_base = OTHER; note = 5-methyl-cytidine
  modified_base 739: mod_base = OTHER; note = Pseudouridine
  modified_base 741..743: mod_base = OTHER; note = 5-methyl-cytidine
  modified_base 744..745: mod_base = OTHER; note = Pseudouridine
  modified_base 746: mod_base = OTHER; note = 5-methyl-cytidine
  modified_base 747..748: mod_base = OTHER; note = Pseudouridine
  modified_base 749: mod_base = OTHER; note = 5-methyl-cytidine
  modified_base 750: mod_base = OTHER; note = Pseudouridine
  modified_base 751: mod_base = OTHER; note = 5-methyl-cytidine
  modified_base 752: mod_base = OTHER; note = Pseudouridine
  modified_base 753..755: mod_base = OTHER; note = 5-methyl-cytidine
  modified_base 756..757: mod_base = OTHER; note = Pseudouridine
  modified_base 759: mod_base = OTHER; note = 5-methyl-cytidine
  modified_base 761..762: mod_base = OTHER; note = 5-methyl-cytidine
  modified_base 763: mod_base = OTHER; note = Pseudouridine
  modified_base 765: mod_base = OTHER; note = Pseudouridine
  modified_base 767..768: mod_base = OTHER; note = 5-methyl-cytidine
  modified_base 769: mod_base = OTHER; note = Pseudouridine
  modified_base 770: mod_base = OTHER; note = 5-methyl-cytidine
  modified_base 771..772: mod_base = OTHER; note = Pseudouridine
  modified_base 775: mod_base = OTHER; note = Pseudouridine
  modified_base 776: mod_base = OTHER; note = 5-methyl-cytidine
  modified_base 777..779: mod_base = OTHER; note = Pseudouridine
  modified_base 783: mod_base = OTHER; note = Pseudouridine
  modified_base 788..789: mod_base = OTHER; note = 5-methyl-cytidine
  modified_base 790: mod_base = OTHER; note = Pseudouridine
  modified_base 794: mod_base = OTHER; note = Pseudouridine
  modified_base 802: mod_base = OTHER; note = 5-methyl-cytidine
  modified_base 805..806: mod_base = OTHER; note = 5-methyl-cytidine
  modified_base 808: mod_base = OTHER; note = 5-methyl-cytidine
  modified_base 809: mod_base = OTHER; note = Pseudouridine
  modified_base 810: mod_base = OTHER; note = 5-methyl-cytidine
  modified_base 814: mod_base = OTHER; note = 5-methyl-cytidine
  modified_base 816: mod_base = OTHER; note = Pseudouridine
  modified_base 818: mod_base = OTHER; note = 5-methyl-cytidine
  modified_base 820: mod_base = OTHER; note = Pseudouridine
  modified_base 821: mod_base = OTHER; note = 5-methyl-cytidine
  modified_base 822: mod_base = OTHER; note = Pseudouridine
  modified_base 829..831: mod_base = OTHER; note = 5-methyl-cytidine
  modified_base 834..835: mod_base = OTHER; note = Pseudouridine
  modified_base 836: mod_base = OTHER; note = 5-methyl-cytidine
  modified_base 838..840: mod_base = OTHER; note = 5-methyl-cytidine
  modified_base 841: mod_base = OTHER; note = Pseudouridine
  modified_base 843..844: mod_base = OTHER; note = Pseudouridine
  modified_base 845: mod_base = OTHER; note = 5-methyl-cytidine
  modified_base 850: mod_base = OTHER; note = Pseudouridine
  modified_base 851: mod_base = OTHER; note = 5-methyl-cytidine
  source 1..852: mol_type = other RNA

SEQ ID NO: 19 - RNA, 437 nt, Homo sapiens
  source 1..437: mol_type = mRNA
aagcttttggaccctcgtacagaagctaatacgactcactatagggaaataagagagaaaagaagagtaagaagaaatataagagccaccatgcctagatcatgttgttcacgatcgggagcgttgttgctggcactcttgctccaagcctcgatggaagtgcggggatggtgccttgagagctcccagtgccaggacctcacaacggagtcgaaccttctggaatgcatccgcgcgtgtaaaccgagggaaggtaaaaggtcctacagcatggagcacttcagatgggggaagcccgtcgggaagaagcggtgaagcgctgccttctgcggggcttgccttctggccatgcccttcttctctcccttgcacctgtacctcttggtctttgaataaagcctgagtaggaaggcggccgctcgagcatgcatctagag

SEQ ID NO: 20 - RNA, 437 nt, synthetic construct; identical to SEQ ID NO: 19
  misc_feature 1..437: note = Description of Artificial Sequence: Synthetic polynucleotide
  modified_base 4: mod_base = OTHER; note = 5-methyl-cytidine
  modified_base 5..8: mod_base = OTHER; note = Pseudouridine
  modified_base 12..14: mod_base = OTHER; note = 5-methyl-cytidine
  modified_base 15: mod_base = OTHER; note = Pseudouridine
  modified_base 16: mod_base = OTHER; note = 5-methyl-cytidine
  modified_base 18: mod_base = OTHER; note = Pseudouridine
  modified_base 20: mod_base = OTHER; note = 5-methyl-cytidine
  modified_base 26: mod_base = OTHER; note = 5-methyl-cytidine
  modified_base 27: mod_base = OTHER; note = Pseudouridine
  modified_base 30: mod_base = OTHER; note = Pseudouridine
  modified_base 32: mod_base = OTHER; note = 5-methyl-cytidine
  modified_base 35: mod_base = OTHER; note = 5-methyl-cytidine
  modified_base 36: mod_base = OTHER; note = Pseudouridine
  modified_base 37: mod_base = OTHER; note = 5-methyl-cytidine
  modified_base 39: mod_base = OTHER; note = 5-methyl-cytidine
  modified_base 40: mod_base = OTHER; note = Pseudouridine
  modified_base 42: mod_base = OTHER; note = Pseudouridine
  modified_base 50: mod_base = OTHER; note = Pseudouridine
  modified_base 68: mod_base = OTHER; note = Pseudouridine
  modified_base 78: mod_base = OTHER; note = Pseudouridine
  modified_base 80: mod_base = OTHER; note = Pseudouridine
  modified_base 86..87: mod_base = OTHER; note = 5-methyl-cytidine
  modified_base 89..90: mod_base = OTHER; note = 5-methyl-cytidine
  modified_base 92: mod_base = OTHER; note = Pseudouridine
  modified_base 94..95: mod_base = OTHER; note = 5-methyl-cytidine
  modified_base 96: mod_base = OTHER; note = Pseudouridine
  modified_base 100: mod_base = OTHER; note = Pseudouridine
  modified_base 101: mod_base = OTHER; note = 5-methyl-cytidine
  modified_base 103: mod_base = OTHER; note = Pseudouridine
  modified_base 105..106: mod_base = OTHER; note = Pseudouridine
  modified_base 108..109: mod_base = OTHER; note = Pseudouridine
  modified_base 110: mod_base = OTHER; note = 5-methyl-cytidine
  modified_base 112: mod_base = OTHER; note = 5-methyl-cytidine
  modified_base 115: mod_base = OTHER; note = Pseudouridine
  modified_base 116: mod_base = OTHER; note = 5-methyl-cytidine
  modified_base 122: mod_base = OTHER; note = 5-methyl-cytidine
  modified_base 124..125: mod_base = OTHER; note = Pseudouridine
  modified_base 127..128: mod_base = OTHER; note = Pseudouridine
  modified_base 130: mod_base = OTHER; note = 5-methyl-cytidine
  modified_base 131: mod_base = OTHER; note = Pseudouridine
  modified_base 134: mod_base = OTHER; note = 5-methyl-cytidine
  modified_base 136: mod_base = OTHER; note = 5-methyl-cytidine
  modified_base 137: mod_base = OTHER; note = Pseudouridine
  modified_base 138: mod_base = OTHER; note = 5-methyl-cytidine
  modified_base 139..140: mod_base = OTHER; note = Pseudouridine
  modified_base 142: mod_base = OTHER; note = 5-methyl-cytidine
  modified_base 143: mod_base = OTHER; note = Pseudouridine
  modified_base 144..145: mod_base = OTHER; note = 5-methyl-cytidine
  modified_base 149..150: mod_base = OTHER; note = 5-methyl-cytidine
  modified_base 151: mod_base = OTHER; note = Pseudouridine
  modified_base 152: mod_base = OTHER; note = 5-methyl-cytidine
  modified_base 155: mod_base = OTHER; note = Pseudouridine
  modified_base 161: mod_base = OTHER; note = Pseudouridine
  modified_base 163: mod_base = OTHER; note = 5-methyl-cytidine
  modified_base 169: mod_base = OTHER; note = Pseudouridine
  modified_base 172: mod_base = OTHER; note = Pseudouridine
  modified_base 174..175: mod_base = OTHER; note = 5-methyl-cytidine
  modified_base 176..177: mod_base = OTHER; note = Pseudouridine
  modified_base 183: mod_base = OTHER; note = 5-methyl-cytidine
  modified_base 184: mod_base = OTHER; note = Pseudouridine
  modified_base 185..187: mod_base = OTHER; note = 5-methyl-cytidine
  modified_base 190: mod_base = OTHER; note = Pseudouridine
  modified_base 192..193: mod_base = OTHER; note = 5-methyl-cytidine
  modified_base 198..199: mod_base = OTHER; note = 5-methyl-cytidine
  modified_base 200: mod_base = OTHER; note = Pseudouridine
  modified_base 201: mod_base = OTHER; note = 5-methyl-cytidine
  modified_base 203: mod_base = OTHER; note = 5-methyl-cytidine
  modified_base 206: mod_base = OTHER; note = 5-methyl-cytidine
  modified_base 211: mod_base = OTHER; note = Pseudouridine
  modified_base 212: mod_base = OTHER; note = 5-methyl-cytidine
  modified_base 216..217: mod_base = OTHER; note = 5-methyl-cytidine
  modified_base 218..219: mod_base = OTHER; note = Pseudouridine
  modified_base 220: mod_base = OTHER; note = 5-methyl-cytidine
  modified_base 221: mod_base = OTHER; note = Pseudouridine
  modified_base 226: mod_base = OTHER; note = Pseudouridine
  modified_base 228: mod_base = OTHER; note = 5-methyl-cytidine
  modified_base 230: mod_base = OTHER; note = Pseudouridine
  modified_base 231..232: mod_base = OTHER; note = 5-methyl-cytidine
  modified_base 234: mod_base = OTHER; note = 5-methyl-cytidine
  modified_base 236: mod_base = OTHER; note = 5-methyl-cytidine
  modified_base 238: mod_base = OTHER; note = Pseudouridine
  modified_base 240: mod_base = OTHER; note = Pseudouridine
  modified_base 244..245: mod_base = OTHER; note = 5-methyl-cytidine
  modified_base 255: mod_base = OTHER; note = Pseudouridine
  modified_base 262: mod_base = OTHER; note = Pseudouridine
  modified_base 263..264: mod_base = OTHER; note = 5-methyl-cytidine
  modified_base 265: mod_base = OTHER; note = Pseudouridine
  modified_base 267: mod_base = OTHER; note = 5-methyl-cytidine
  modified_base 270: mod_base = OTHER; note = 5-methyl-cytidine
  modified_base 272: mod_base = OTHER; note = Pseudouridine
  modified_base 277: mod_base = OTHER; note = 5-methyl-cytidine
  modified_base 279: mod_base = OTHER; note = 5-methyl-cytidine
  modified_base 280..281: mod_base = OTHER; note = Pseudouridine
  modified_base 282: mod_base = OTHER; note = 5-methyl-cytidine
  modified_base 286: mod_base = OTHER; note = Pseudouridine
  modified_base 295..297: mod_base = OTHER; note = 5-methyl-cytidine
  modified_base 299: mod_base = OTHER; note = Pseudouridine
  modified_base 300: mod_base = OTHER; note = 5-methyl-cytidine
  modified_base 310: mod_base = OTHER; note = 5-methyl-cytidine
  modified_base 313: mod_base = OTHER; note = Pseudouridine
  modified_base 318: mod_base = OTHER; note = 5-methyl-cytidine
  modified_base 320: mod_base = OTHER; note = 5-methyl-cytidine
  modified_base 321: mod_base = OTHER; note = Pseudouridine
  modified_base 323..324: mod_base = OTHER; note = 5-methyl-cytidine
  modified_base 325..326: mod_base = OTHER; note = Pseudouridine
  modified_base 327: mod_base = OTHER; note = 5-methyl-cytidine
  modified_base 328: mod_base = OTHER; note = Pseudouridine
  modified_base 330: mod_base = OTHER; note = 5-methyl-cytidine
  modified_base 335: mod_base = OTHER; note = 5-methyl-cytidine
  modified_base 336..337: mod_base = OTHER; note = Pseudouridine
  modified_base 339..340: mod_base = OTHER; note = 5-methyl-cytidine
  modified_base 341..342: mod_base = OTHER; note = Pseudouridine
  modified_base 343: mod_base = OTHER; note = 5-methyl-cytidine
  modified_base 344: mod_base = OTHER; note = Pseudouridine
  modified_base 347..348: mod_base = OTHER; note = 5-methyl-cytidine
  modified_base 350: mod_base = OTHER; note = Pseudouridine
  modified_base 352..354: mod_base = OTHER; note = 5-methyl-cytidine
  modified_base 355..356: mod_base = OTHER; note = Pseudouridine
  modified_base 357: mod_base = OTHER; note = 5-methyl-cytidine
  modified_base 358..359: mod_base = OTHER; note = Pseudouridine
  modified_base 360: mod_base = OTHER; note = 5-methyl-cytidine
  modified_base 361: mod_base = OTHER; note = Pseudouridine
  modified_base 362: mod_base = OTHER; note = 5-methyl-cytidine
  modified_base 363: mod_base = OTHER; note = Pseudouridine
  modified_base 364..366: mod_base = OTHER; note = 5-methyl-cytidine
  modified_base 367..368: mod_base = OTHER; note = Pseudouridine
  modified_base 370: mod_base = OTHER; note = 5-methyl-cytidine
  modified_base 372..373: mod_base = OTHER; note = 5-methyl-cytidine
  modified_base 374: mod_base = OTHER; note = Pseudouridine
  modified_base 376: mod_base = OTHER; note = Pseudouridine
  modified_base 378..379: mod_base = OTHER; note = 5-methyl-cytidine
  modified_base 380: mod_base = OTHER; note = Pseudouridine
  modified_base 381: mod_base = OTHER; note = 5-methyl-cytidine
  modified_base 382..383: mod_base = OTHER; note = Pseudouridine
  modified_base 386: mod_base = OTHER; note = Pseudouridine
  modified_base 387: mod_base = OTHER; note = 5-methyl-cytidine
  modified_base 388..390: mod_base = OTHER; note = Pseudouridine
  modified_base 394: mod_base = OTHER; note = Pseudouridine
  modified_base 399..400: mod_base = OTHER; note = 5-methyl-cytidine
  modified_base 401: mod_base = OTHER; note = Pseudouridine
  modified_base 405: mod_base = OTHER; note = Pseudouridine
  modified_base 413: mod_base = OTHER; note = 5-methyl-cytidine
  modified_base 416..417: mod_base = OTHER; note = 5-methyl-cytidine
  modified_base 419: mod_base = OTHER; note = 5-methyl-cytidine
  modified_base 420: mod_base = OTHER; note = Pseudouridine
  modified_base 421: mod_base = OTHER; note = 5-methyl-cytidine
  modified_base 425: mod_base = OTHER; note = 5-methyl-cytidine
  modified_base 427: mod_base = OTHER; note = Pseudouridine
  modified_base 429: mod_base = OTHER; note = 5-methyl-cytidine
  modified_base 431: mod_base = OTHER; note = Pseudouridine
  modified_base 432: mod_base = OTHER; note = 5-methyl-cytidine
  modified_base 433: mod_base = OTHER; note = Pseudouridine
  source 1..437: mol_type = other RNA

SEQ ID NO: 21 - RNA, 563 nt, Mus sp.
  source 1..563: mol_type = mRNA
agcttttggaccctcgtacagaagctaatacgactcactatagggaaataagagagaaaagaagagtaagaagaaatataagagccaccatggcgctctggtgaggtttctgcctttgttggccttgctgttcctctgggagtcccaccccacacaggcgtttgtgaagcagcacttgtgcgggtcgcatctggtggaggcactttatctcgtctgcggcgagcgaggattcttctacaccccgatgagcagacgcgaagtggaagatccgcaagtcgcgcagctcgaacttgggggtggtcccggagccggagacttgcaaactctcgctctcgaggtagcgcagcagaaacggggtatcgtagaccagtgttgcacgtcgatctgttcactttatcaacttgagaactactgtaattgaagcgctgccttctgcggggcttgccttctggccatgcccttcttctctcccttgcacctgtacctcttggtctttgaataaagcctgagtaggaaggcggccgctcgagcatgcatctagagggcccaattcgccctattcg

SEQ ID NO: 22 - RNA, 563 nt, synthetic construct; identical to SEQ ID NO: 21
  misc_feature 1..563: note = Description of Artificial Sequence: Synthetic polynucleotide
  modified_base 3: mod_base = OTHER; note = 5-methyl-cytidine
  modified_base 4..7: mod_base = OTHER; note = Pseudouridine
  modified_base 11..13: mod_base = OTHER; note = 5-methyl-cytidine
  modified_base 14: mod_base = OTHER; note = Pseudouridine
  modified_base 15: mod_base = OTHER; note = 5-methyl-cytidine
  modified_base 17: mod_base = OTHER; note = Pseudouridine
  modified_base 19: mod_base = OTHER; note = 5-methyl-cytidine
  modified_base 25: mod_base = OTHER; note = 5-methyl-cytidine
  modified_base 26: mod_base = OTHER; note = Pseudouridine
  modified_base 29: mod_base = OTHER; note = Pseudouridine
  modified_base 31: mod_base = OTHER; note = 5-methyl-cytidine
  modified_base 34: mod_base = OTHER; note = 5-methyl-cytidine
  modified_base 35: mod_base = OTHER; note = Pseudouridine
  modified_base 36: mod_base = OTHER; note = 5-methyl-cytidine
  modified_base 38: mod_base = OTHER; note = 5-methyl-cytidine
  modified_base 39: mod_base = OTHER; note = Pseudouridine
  modified_base 41: mod_base = OTHER; note = Pseudouridine
  modified_base 49: mod_base = OTHER; note = Pseudouridine
  modified_base 67: mod_base = OTHER; note = Pseudouridine
  modified_base 77: mod_base = OTHER; note = Pseudouridine
  modified_base 79: mod_base = OTHER; note = Pseudouridine
  modified_base 85..86: mod_base = OTHER; note = 5-methyl-cytidine
  modified_base 88..89: mod_base = OTHER; note = 5-methyl-cytidine
  modified_base 91: mod_base = OTHER; note = Pseudouridine
  modified_base 94: mod_base = OTHER; note = 5-methyl-cytidine
  modified_base 96: mod_base = OTHER; note = 5-methyl-cytidine
  modified_base 97: mod_base = OTHER; note = Pseudouridine
  modified_base 98: mod_base = OTHER; note = 5-methyl-cytidine
  modified_base 99: mod_base = OTHER; note = Pseudouridine
  modified_base 102: mod_base = OTHER; note = Pseudouridine
  modified_base 107..109: mod_base = OTHER; note = Pseudouridine
  modified_base 110: mod_base = OTHER; note = 5-methyl-cytidine
  modified_base 111: mod_base = OTHER; note = Pseudouridine
  modified_base 113..114: mod_base = OTHER; note = 5-methyl-cytidine
  modified_base 115..117: mod_base = OTHER; note = Pseudouridine
  modified_base 119..120: mod_base = OTHER; note = Pseudouridine
  modified_base 123..124: mod_base = OTHER; note = 5-methyl-cytidine
  modified_base 125..126: mod_base = OTHER; note = Pseudouridine
  modified_base 128: mod_base = OTHER; note = 5-methyl-cytidine
  modified_base 129: mod_base = OTHER; note = Pseudouridine
  modified_base 131..132: mod_base = OTHER; note = Pseudouridine
  modified_base 133..134: mod_base = OTHER; note = 5-methyl-cytidine
  modified_base 135: mod_base = OTHER; note = Pseudouridine
  modified_base 136: mod_base = OTHER; note = 5-methyl-cytidine
  modified_base 137: mod_base = OTHER; note = Pseudouridine
  modified_base 143: mod_base = OTHER; note = Pseudouridine
  modified_base 144..146: mod_base = OTHER; note = 5-methyl-cytidine
  modified_base 148..151: mod_base = OTHER; note = 5-methyl-cytidine
  modified_base 153: mod_base = OTHER; note = 5-methyl-cytidine
  modified_base 155: mod_base = OTHER; note = 5-methyl-cytidine
  modified_base 159: mod_base = OTHER; note = 5-methyl-cytidine
  modified_base 161..163: mod_base = OTHER; note = Pseudouridine
  modified_base 165: mod_base = OTHER; note = Pseudouridine
  modified_base 170: mod_base = OTHER; note = 5-methyl-cytidine
  modified_base 173: mod_base = OTHER; note = 5-methyl-cytidine
  modified_base 175: mod_base = OTHER; note = 5-methyl-cytidine
  modified_base 176..177: mod_base = OTHER; note = Pseudouridine
  modified_base 179: mod_base = OTHER; note = Pseudouridine
  modified_base 181: mod_base = OTHER; note = 5-methyl-cytidine
  modified_base 185: mod_base = OTHER; note = Pseudouridine
  modified_base 186: mod_base = OTHER; note = 5-methyl-cytidine
  modified_base 188: mod_base = OTHER; note = 5-methyl-cytidine
  modified_base 190: mod_base = OTHER; note = Pseudouridine
  modified_base 191: mod_base = OTHER; note = 5-methyl-cytidine
  modified_base 192: mod_base = OTHER; note = Pseudouridine
  modified_base 195: mod_base = OTHER; note = Pseudouridine
  modified_base 201: mod_base = OTHER; note = 5-methyl-cytidine
  modified_base 203: mod_base = OTHER; note = 5-methyl-cytidine
  modified_base 204..206: mod_base = OTHER; note = Pseudouridine
  modified_base 208: mod_base = OTHER; note = Pseudouridine
  modified_base 209: mod_base = OTHER; note = 5-methyl-cytidine
  modified_base 210: mod_base = OTHER; note = Pseudouridine
  modified_base 211: mod_base = OTHER; note = 5-methyl-cytidine
  modified_base 213: mod_base = OTHER; note = Pseudouridine
  modified_base 214: mod_base = OTHER; note = 5-methyl-cytidine
  modified_base 215: mod_base = OTHER; note = Pseudouridine
  modified_base 217: mod_base = OTHER; note = 5-methyl-cytidine
  modified_base 220: mod_base = OTHER; note = 5-methyl-cytidine
  modified_base 224: mod_base = OTHER; note = 5-methyl-cytidine
  modified_base 230..231: mod_base = OTHER; note = Pseudouridine
  modified_base 232: mod_base = OTHER; note = 5-methyl-cytidine
  modified_base 233..234: mod_base = OTHER; note = Pseudouridine
  modified_base 235: mod_base = OTHER; note = 5-methyl-cytidine
  modified_base 236: mod_base = OTHER; note = Pseudouridine
  modified_base 238: mod_base = OTHER; note = 5-methyl-cytidine
  modified_base 240..243: mod_base = OTHER; note = 5-methyl-cytidine
  modified_base 246: mod_base = OTHER; note = Pseudouridine
  modified_base 250: mod_base = OTHER; note = 5-methyl-cytidine
  modified_base 254: mod_base = OTHER; note = 5-methyl-cytidine
  modified_base 256: mod_base = OTHER; note = 5-methyl-cytidine
  modified_base 261: mod_base = OTHER; note = Pseudouridine
  modified_base 268: mod_base = OTHER; note = Pseudouridine
  modified_base 269..270: mod_base = OTHER; note = 5-methyl-cytidine
  modified_base 272: mod_base = OTHER; note = 5-methyl-cytidine
  modified_base 276: mod_base = OTHER; note = Pseudouridine
  modified_base 277: mod_base = OTHER; note = 5-methyl-cytidine
  modified_base 279: mod_base = OTHER; note = 5-methyl-cytidine
  modified_base 281: mod_base = OTHER; note = 5-methyl-cytidine
  modified_base 284: mod_base = OTHER; note = 5-methyl-cytidine
  modified_base 285: mod_base = OTHER; note = Pseudouridine
  modified_base 286: mod_base = OTHER; note = 5-methyl-cytidine
  modified_base 290: mod_base = OTHER; note = 5-methyl-cytidine
  modified_base 291..292: mod_base = OTHER; note = Pseudouridine
  modified_base 298: mod_base = OTHER; note = Pseudouridine
  modified_base 301: mod_base = OTHER; note = Pseudouridine
  modified_base 302..304: mod_base = OTHER; note = 5-methyl-cytidine
  modified_base 309..310: mod_base = OTHER; note = 5-methyl-cytidine
  modified_base 316: mod_base = OTHER; note = 5-methyl-cytidine
  modified_base 317..318: mod_base = OTHER; note = Pseudouridine
  modified_base 320: mod_base = OTHER; note = 5-methyl-cytidine
  modified_base 324: mod_base = OTHER; note = 5-methyl-cytidine
  modified_base 325: mod_base = OTHER; note = Pseudouridine
  modified_base 326: mod_base = OTHER; note = 5-methyl-cytidine
  modified_base 327: mod_base = OTHER; note = Pseudouridine
  modified_base 328: mod_base = OTHER; note = 5-methyl-cytidine
  modified_base 330: mod_base = OTHER; note = 5-methyl-cytidine
  modified_base 331: mod_base = OTHER; note = Pseudouridine
  modified_base 332: mod_base = OTHER; note = 5-methyl-cytidine
  modified_base 333: mod_base = OTHER; note = Pseudouridine
  modified_base 334: mod_base = OTHER; note = 5-methyl-cytidine
  modified_base 339: mod_base = OTHER; note = Pseudouridine
  modified_base 342: mod_base = OTHER; note = 5-methyl-cytidine
  modified_base 344: mod_base = OTHER; note = 5-methyl-cytidine
  modified_base 347: mod_base = OTHER; note = 5-methyl-cytidine
  modified_base 353: mod_base = OTHER; note = 5-methyl-cytidine
  modified_base 358: mod_base = OTHER; note = Pseudouridine
  modified_base 360: mod_base = OTHER; note = Pseudouridine
  modified_base 361: mod_base = OTHER; note = 5-methyl-cytidine
  modified_base 363: mod_base = OTHER; note = Pseudouridine
  modified_base 367..368: mod_base = OTHER; note = 5-methyl-cytidine
  modified_base 371: mod_base = OTHER; note = Pseudouridine
  modified_base 373..374: mod_base = OTHER; note = Pseudouridine
  modified_base 376: mod_base = OTHER; note = 5-methyl-cytidine
  modified_base 378: mod_base = OTHER; note = 5-methyl-cytidine
  modified_base 380: mod_base = OTHER; note = Pseudouridine
  modified_base 381: mod_base = OTHER; note = 5-methyl-cytidine
  modified_base 384: mod_base = OTHER; note = Pseudouridine
  modified_base 385: mod_base = OTHER; note = 5-methyl-cytidine
  modified_base 386: mod_base = OTHER; note = Pseudouridine
  modified_base 388..389: mod_base = OTHER; note = Pseudouridine
  modified_base 390: mod_base = OTHER; note = 5-methyl-cytidine
  modified_base 392: mod_base = OTHER; note = 5-methyl-cytidine
  modified_base 393..395: mod_base = OTHER; note = Pseudouridine
  modified_base 397: mod_base = OTHER; note = Pseudouridine
  modified_base 398: mod_base = OTHER; note = 5-methyl-cytidine
  modified_base 401: mod_base = OTHER; note = 5-methyl-cytidine
  modified_base 402..403: mod_base = OTHER; note = Pseudouridine
  modified_base 409: mod_base = OTHER; note = 5-methyl-cytidine
  modified_base 410: mod_base = OTHER; note = Pseudouridine
  modified_base 412: mod_base = OTHER; note = 5-methyl-cytidine
  modified_base 413: mod_base = OTHER; note = Pseudouridine
  modified_base 415: mod_base = OTHER; note = Pseudouridine
  modified_base 418..419: mod_base = OTHER; note = Pseudouridine
  modified_base 424: mod_base = OTHER; note = 5-methyl-cytidine
  modified_base 426: mod_base = OTHER; note = 5-methyl-cytidine
  modified_base 427: mod_base = OTHER; note = Pseudouridine
  modified_base 429..430: mod_base = OTHER; note = 5-methyl-cytidine
  modified_base 431..432: mod_base = OTHER; note = Pseudouridine
  modified_base 433: mod_base = OTHER; note = 5-methyl-cytidine
  modified_base 434: mod_base = OTHER; note = Pseudouridine
  modified_base 436: mod_base = OTHER; note = 5-methyl-cytidine
  modified_base 441: mod_base = OTHER; note = 5-methyl-cytidine
  modified_base 442..443: mod_base = OTHER; note = Pseudouridine
  modified_base 445..446: mod_base = OTHER; note = 5-methyl-cytidine
  modified_base 447..448: mod_base = OTHER; note = Pseudouridine
  modified_base 449: mod_base = OTHER; note = 5-methyl-cytidine
  modified_base 450: mod_base = OTHER; note = Pseudouridine
  modified_base 453..454: mod_base = OTHER; note = 5-methyl-cytidine
  modified_base 456: mod_base = OTHER; note = Pseudouridine
  modified_base 458..460: mod_base = OTHER; note = 5-methyl-cytidine
  modified_base 461..462: mod_base = OTHER; note = Pseudouridine
  modified_base 463: mod_base = OTHER; note = 5-methyl-cytidine
  modified_base 464..465: mod_base = OTHER; note = Pseudouridine
  modified_base 466: mod_base = OTHER; note = 5-methyl-cytidine
  modified_base 467: mod_base = OTHER; note = Pseudouridine
  modified_base 468: mod_base = OTHER; note = 5-methyl-cytidine
  modified_base 469: mod_base = OTHER; note = Pseudouridine
  modified_base 470..472: mod_base = OTHER; note = 5-methyl-cytidine
  modified_base 473..474: mod_base = OTHER; note = Pseudouridine
  modified_base 476: mod_base = OTHER; note = 5-methyl-cytidine
  modified_base 478..479: mod_base = OTHER; note = 5-methyl-cytidine
  modified_base 480: mod_base = OTHER; note = Pseudouridine
  modified_base 482: mod_base = OTHER; note = Pseudouridine
  modified_base 484..485: mod_base = OTHER; note = 5-methyl-cytidine
  modified_base 486: mod_base = OTHER; note = Pseudouridine
  modified_base 487: mod_base = OTHER; note = 5-methyl-cytidine
  modified_base 488..489: mod_base = OTHER; note = Pseudouridine
  modified_base 492: mod_base = OTHER; note = Pseudouridine
  modified_base 493: mod_base = OTHER; note = 5-methyl-cytidine
  modified_base 494..496: mod_base = OTHER; note = Pseudouridine
  modified_base 500: mod_base = OTHER; note = Pseudouridine
  modified_base 505..506: mod_base = OTHER; note = 5-methyl-cytidine
  modified_base 507: mod_base = OTHER; note = Pseudouridine
  modified_base 511: mod_base = OTHER; note = Pseudouridine
  modified_base 519: mod_base = OTHER; note = 5-methyl-cytidine
  modified_base 522..523: mod_base = OTHER; note = 5-methyl-cytidine
  modified_base 525: mod_base = OTHER; note = 5-methyl-cytidine
  modified_base 526: mod_base = OTHER; note = Pseudouridine
  modified_base 527: mod_base = OTHER; note = 5-methyl-cytidine
  modified_base 531: mod_base = OTHER; note = 5-methyl-cytidine
  modified_base 533: mod_base = OTHER; note = Pseudouridine
  modified_base 535: mod_base = OTHER; note = 5-methyl-cytidine
  modified_base 537: mod_base = OTHER; note = Pseudouridine
  modified_base 538: mod_base = OTHER; note = 5-methyl-cytidine
  modified_base 539: mod_base = OTHER; note = Pseudouridine
  modified_base 546..548: mod_base = OTHER; note = 5-methyl-cytidine
  modified_base 551..552: mod_base = OTHER; note = Pseudouridine
  modified_base 553: mod_base = OTHER; note = 5-methyl-cytidine
  modified_base 555..557: mod_base = OTHER; note = 5-methyl-cytidine
  modified_base 558: mod_base = OTHER; note = Pseudouridine
  modified_base 560..561: mod_base = OTHER; note = Pseudouridine
  modified_base 562: mod_base = OTHER; note = 5-methyl-cytidine
  source 1..563: mol_type = other RNA

SEQ ID NO: 23 - RNA, 567 nt, Homo sapiens
  source 1..567: mol_type = mRNA
agcttttggaccctcgtacagaagctaatacgactcactatagggaaataagagagaaaagaagagtaagaagaaatataagagccaccatggctctctggatgcggcttcttccactgctcgccctcttggcgttgtggggacccgaccctgcagcagcgtttgtgaatcagcacctctgcgggtcccatctggtcgaagccctttaccttgtgtgtggcgagcgcgggttcttctacacgcccaagacaaggcgcgaggcggaggatctccaagtagggcaggtggaattgggagggggaccgggagccggttcactccagcccctggcgttggaggggtcgctgcagaaaagaggtattgtcgaacagtgttgcactagcatctgctcgctgtatcaacttgagaactattgtaactgaagcgctgccttctgcggggcttgccttctggccatgcccttcttctctcccttgcacctgtacctcttggtctttgaataaagcctgagtaggaaggcggccgctcgagcatgcatctagagggcccaattcgccctattcgaag

SEQ ID NO: 24 - RNA, 567 nt, synthetic construct; identical to SEQ ID NO: 23
  misc_feature 1..567: note = Description of Artificial Sequence: Synthetic polynucleotide
  modified_base 3: mod_base = OTHER; note = 5-methyl-cytidine
  modified_base 4..7: mod_base = OTHER; note = Pseudouridine
  modified_base 11..13: mod_base = OTHER; note = 5-methyl-cytidine
  modified_base 14: mod_base = OTHER; note = Pseudouridine
  modified_base 15: mod_base = OTHER; note = 5-methyl-cytidine
  modified_base 17: mod_base = OTHER; note = Pseudouridine
  modified_base 19: mod_base = OTHER; note = 5-methyl-cytidine
  modified_base 25: mod_base = OTHER; note = 5-methyl-cytidine
  modified_base 26: mod_base = OTHER; note = Pseudouridine
  modified_base 29: mod_base = OTHER; note = Pseudouridine
  modified_base 31: mod_base = OTHER; note = 5-methyl-cytidine
  modified_base 34: mod_base = OTHER; note = 5-methyl-cytidine
  modified_base 35: mod_base = OTHER; note = Pseudouridine
  modified_base 36: mod_base = OTHER; note = 5-methyl-cytidine
  modified_base 38: mod_base = OTHER; note = 5-methyl-cytidine
  modified_base 39: mod_base = OTHER; note = Pseudouridine
  modified_base 41: mod_base = OTHER; note = Pseudouridine
  modified_base 49: mod_base = OTHER; note = Pseudouridine
  modified_base 67: mod_base = OTHER; note = Pseudouridine
  modified_base 77: mod_base = OTHER; note = Pseudouridine
  modified_base 79: mod_base = OTHER; note = Pseudouridine
  modified_base 85..86: mod_base = OTHER; note = 5-methyl-cytidine
  modified_base 88..89: mod_base = OTHER; note = 5-methyl-cytidine
  modified_base 91: mod_base = OTHER; note = Pseudouridine
  modified_base 94: mod_base = OTHER; note = 5-methyl-cytidine
  modified_base 95: mod_base = OTHER; note = Pseudouridine
  modified_base 96: mod_base = OTHER; note = 5-methyl-cytidine
  modified_base 97: mod_base = OTHER; note = Pseudouridine
  modified_base 98: mod_base = OTHER; note = 5-methyl-cytidine
  modified_base 99: mod_base = OTHER; note = Pseudouridine
  modified_base 103: mod_base = OTHER; note = Pseudouridine
  modified_base 105: mod_base = OTHER; note = 5-methyl-cytidine
  modified_base 108: mod_base = OTHER; note = 5-methyl-cytidine
  modified_base 109..110: mod_base = OTHER; note = Pseudouridine
  modified_base 111: mod_base = OTHER; note = 5-methyl-cytidine
  modified_base 112..113: mod_base = OTHER; note = Pseudouridine
  modified_base 114..115: mod_base = OTHER; note = 5-methyl-cytidine
  modified_base 117: mod_base = OTHER; note = 5-methyl-cytidine
  modified_base 118: mod_base = OTHER; note = Pseudouridine
  modified_base 120: mod_base = OTHER; note = 5-methyl-cytidine
  modified_base 121: mod_base = OTHER; note = Pseudouridine
  modified_base 122: mod_base = OTHER; note = 5-methyl-cytidine
  modified_base 124..126: mod_base = OTHER; note = 5-methyl-cytidine
  modified_base 127: mod_base = OTHER; note = Pseudouridine
  modified_base 128: mod_base = OTHER; note = 5-methyl-cytidine
  modified_base 129..130: mod_base = OTHER; note = Pseudouridine
  modified_base 133: mod_base = OTHER; note = 5-methyl-cytidine
  modified_base 135..136: mod_base = OTHER; note = Pseudouridine
  modified_base 138: mod_base = OTHER; note = Pseudouridine
  modified_base 144..146: mod_base = OTHER; note = 5-methyl-cytidine
  modified_base 149..151: mod_base = OTHER; note = 5-methyl-cytidine
  modified_base 152: mod_base = OTHER; note = Pseudouridine
  modified_base 154: mod_base = OTHER; note = 5-methyl-cytidine
  modified_base 157: mod_base = OTHER; note = 5-methyl-cytidine
  modified_base 160: mod_base = OTHER; note = 5-methyl-cytidine
  modified_base 162..164: mod_base = OTHER; note = Pseudouridine
  modified_base 166: mod_base = OTHER; note = Pseudouridine
  modified_base 170: mod_base = OTHER; note = Pseudouridine
  modified_base 171: mod_base = OTHER; note = 5-methyl-cytidine
  modified_base 174: mod_base = OTHER; note = 5-methyl-cytidine
  modified_base 176..177: mod_base = OTHER; note = 5-methyl-cytidine
  modified_base 178: mod_base = OTHER; note = Pseudouridine
  modified_base 179: mod_base = OTHER; note = 5-methyl-cytidine
  modified_base 180: mod_base = OTHER; note = Pseudouridine
  modified_base 182: mod_base = OTHER; note = 5-methyl-cytidine
  modified_base 186: mod_base = OTHER; note = Pseudouridine
  modified_base 187..189: mod_base = OTHER; note = 5-methyl-cytidine
  modified_base 191: mod_base = OTHER; note = Pseudouridine
  modified_base 192: mod_base = OTHER; note = 5-methyl-cytidine
  modified_base 193: mod_base = OTHER; note = Pseudouridine
  modified_base 196: mod_base = OTHER; note = Pseudouridine
  modified_base 197: mod_base = OTHER; note = 5-methyl-cytidine
  modified_base 202..204: mod_base = OTHER; note = 5-methyl-cytidine
  modified_base 205..207: mod_base = OTHER; note = Pseudouridine
  modified_base 209..210: mod_base = OTHER; note = 5-methyl-cytidine
  modified_base 211..212: mod_base = OTHER; note = Pseudouridine
  modified_base 214: mod_base = OTHER; note = Pseudouridine
  modified_base 216: mod_base = OTHER; note = Pseudouridine
  modified_base 218: mod_base = OTHER; note = Pseudouridine
  modified_base 221: mod_base = OTHER; note = 5-methyl-cytidine
  modified_base 225: mod_base = OTHER; note = 5-methyl-cytidine
  modified_base 227: mod_base = OTHER; note = 5-methyl-cytidine
  modified_base 231..232: mod_base = OTHER; note = Pseudouridine
  modified_base 233: mod_base = OTHER; note = 5-methyl-cytidine
  modified_base 234..235: mod_base = OTHER; note = Pseudouridine
  modified_base 236: mod_base = OTHER; note = 5-methyl-cytidine
  modified_base 237: mod_base = OTHER; note = Pseudouridine
  modified_base 239: mod_base = OTHER; note = 5-methyl-cytidine
  modified_base 241: mod_base = OTHER; note = 5-methyl-cytidine
  modified_base 243..245: mod_base = OTHER; note = 5-methyl-cytidine
  modified_base 250: mod_base = OTHER; note = 5-methyl-cytidine
  modified_base 255: mod_base = OTHER; note = 5-methyl-cytidine
  modified_base 257: mod_base = OTHER; note = 5-methyl-cytidine
  modified_base 262: mod_base = OTHER; note = 5-methyl-cytidine
  modified_base 269: mod_base = OTHER; note = Pseudouridine
  modified_base 270: mod_base = OTHER; note = 5-methyl-cytidine
  modified_base 271: mod_base = OTHER; note = Pseudouridine
  modified_base 272..273: mod_base = OTHER; note = 5-methyl-cytidine
  modified_base 277: mod_base = OTHER; note = Pseudouridine
  modified_base 282: mod_base = OTHER; note = 5-methyl-cytidine
  modified_base 286: mod_base = OTHER; note = Pseudouridine
  modified_base 291..292: mod_base = OTHER; note = Pseudouridine
  modified_base 303..304: mod_base = OTHER; note = 5-methyl-cytidine
  modified_base 310..311: mod_base = OTHER; note = 5-methyl-cytidine
  modified_base 314..315: mod_base = OTHER; note = Pseudouridine
  modified_base 316: mod_base = OTHER; note = 5-methyl-cytidine
  modified_base 318: mod_base = OTHER; note = 5-methyl-cytidine
  modified_base 319: mod_base = OTHER; note = Pseudouridine
  modified_base 320..321: mod_base = OTHER; note = 5-methyl-cytidine
  modified_base 324..327: mod_base = OTHER; note = 5-methyl-cytidine
  modified_base 328: mod_base = OTHER; note = Pseudouridine
  modified_base 331: mod_base = OTHER; note = 5-methyl-cytidine
  modified_base 333..334: mod_base = OTHER; note = Pseudouridine
  modified_base 342: mod_base = OTHER; note = Pseudouridine
  modified_base 343: mod_base = OTHER; note = 5-methyl-cytidine
  modified_base 345: mod_base = OTHER; note = 5-methyl-cytidine
  modified_base 346: mod_base = OTHER; note = Pseudouridine
  modified_base 348: mod_base = OTHER; note = 5-methyl-cytidine
  modified_base 359: mod_base = OTHER; note = Pseudouridine
  modified_base 361..362: mod_base = OTHER; note = Pseudouridine
  modified_base 364: mod_base = OTHER; note = Pseudouridine
  modified_base 365: mod_base = OTHER; note = 5-methyl-cytidine
  modified_base 369: mod_base = OTHER; note = 5-methyl-cytidine
  modified_base 372: mod_base = OTHER; note = Pseudouridine
  modified_base 374..375: mod_base = OTHER; note = Pseudouridine
  modified_base 377: mod_base = OTHER; note = 5-methyl-cytidine
  modified_base 379: mod_base = OTHER; note = 5-methyl-cytidine
  modified_base 380: mod_base = OTHER; note = Pseudouridine
  modified_base 383: mod_base = OTHER; note = 5-methyl-cytidine
  modified_base 385: mod_base = OTHER; note = Pseudouridine
  modified_base 386: mod_base = OTHER; note = 5-methyl-cytidine
  modified_base 387: mod_base = OTHER; note = Pseudouridine
  modified_base 389: mod_base = OTHER; note = 5-methyl-cytidine
  modified_base 390: mod_base = OTHER; note = Pseudouridine
  modified_base 391: mod_base = OTHER; note = 5-methyl-cytidine
  modified_base 393: mod_base = OTHER; note = 5-methyl-cytidine
  modified_base 394: mod_base = OTHER; note = Pseudouridine
  modified_base 396: mod_base = OTHER; note = Pseudouridine
  modified_base 398: mod_base = OTHER; note = Pseudouridine
  modified_base 399: mod_base = OTHER; note = 5-methyl-cytidine
  modified_base 402: mod_base = OTHER; note = 5-methyl-cytidine
  modified_base 403..404: mod_base = OTHER; note = Pseudouridine
  modified_base 410: mod_base = OTHER; note = 5-methyl-cytidine
  modified_base 411: mod_base = OTHER; note = Pseudouridine
  modified_base 413..414: mod_base = OTHER; note = Pseudouridine
  modified_base 416: mod_base = OTHER; note = Pseudouridine
  modified_base 419: mod_base = OTHER; note = 5-methyl-cytidine
  modified_base 420: mod_base = OTHER; note = Pseudouridine
  modified_base 425: mod_base = OTHER; note = 5-methyl-cytidine
  modified_base 427: mod_base = OTHER; note = 5-methyl-cytidine
  modified_base 428: mod_base = OTHER; note = Pseudouridine
  modified_base 430..431: mod_base = OTHER; note = 5-methyl-cytidine
  modified_base 432..433: mod_base = OTHER; note = Pseudouridine
  modified_base 434: mod_base = OTHER; note = 5-methyl-cytidine
  modified_base 435: mod_base = OTHER; note = Pseudouridine
  modified_base 437: mod_base = OTHER; note = 5-methyl-cytidine
  modified_base 442: mod_base = OTHER; note = 5-methyl-cytidine
  modified_base 443..444: mod_base = OTHER; note = Pseudouridine
  modified_base 446..447: mod_base = OTHER; note = 5-methyl-cytidine
  modified_base 448..449: mod_base = OTHER; note = Pseudouridine
  modified_base 450: mod_base = OTHER; note = 5-methyl-cytidine
  modified_base 451: mod_base = OTHER; note = Pseudouridine
  modified_base 454..455: mod_base = OTHER; note = 5-methyl-cytidine
  modified_base 457: mod_base = OTHER; note = Pseudouridine
  modified_base 459..461: mod_base = OTHER; note = 5-methyl-cytidine
  modified_base 462..463: mod_base = OTHER; note = Pseudouridine
  modified_base 464: mod_base = OTHER; note = 5-methyl-cytidine
  modified_base 465..466: mod_base = OTHER; note = Pseudouridine
  modified_base 467: mod_base = OTHER; note = 5-methyl-cytidine
  modified_base 468: mod_base = OTHER; note = Pseudouridine
  modified_base 469: mod_base = OTHER; note = 5-methyl-cytidine
  modified_base 470: mod_base = OTHER; note = Pseudouridine
  modified_base 471..473: mod_base = OTHER; note = 5-methyl-cytidine
  modified_base 474..475: mod_base = OTHER; note = Pseudouridine
  modified_base 477: mod_base = OTHER; note = 5-methyl-cytidine
  modified_base 479..480: mod_base = OTHER; note = 5-methyl-cytidine
  modified_base 481: mod_base = OTHER; note = Pseudouridine
  modified_base 483: mod_base = OTHER; note = Pseudouridine
  modified_base 485..486: mod_base = OTHER; note = 5-methyl-cytidine
  modified_base 487: mod_base = OTHER; note = Pseudouridine
  modified_base 488: mod_base = OTHER; note = 5-methyl-cytidine
  modified_base 489..490: mod_base = OTHER; note = Pseudouridine
  modified_base 493: mod_base = OTHER; note = Pseudouridine
  modified_base 494: mod_base = OTHER; note = 5-methyl-cytidine
  modified_base 495..497: mod_base = OTHER; note = Pseudouridine
  modified_base 501: mod_base = OTHER; note = Pseudouridine
  modified_base 506..507: mod_base = OTHER; note = 5-methyl-cytidine
  modified_base 508: mod_base = OTHER; note = Pseudouridine
  modified_base 512: mod_base = OTHER; note = Pseudouridine
  modified_base 520: mod_base = OTHER; note = 5-methyl-cytidine
  modified_base 523..524: mod_base = OTHER; note = 5-methyl-cytidine
  modified_base 526: mod_base = OTHER; note = 5-methyl-cytidine
  modified_base 527: mod_base = OTHER; note = Pseudouridine
  modified_base 528: mod_base = OTHER; note = 5-methyl-cytidine
  modified_base 532: mod_base = OTHER; note = 5-methyl-cytidine
  modified_base 534: mod_base = OTHER; note = Pseudouridine
  modified_base 536: mod_base = OTHER; note = 5-methyl-cytidine
  modified_base 538: mod_base = OTHER; note = Pseudouridine
  modified_base 539: mod_base = OTHER; note = 5-methyl-cytidine
  modified_base 540: mod_base = OTHER; note = Pseudouridine
  modified_base 547..549: mod_base = OTHER; note = 5-methyl-cytidine
  modified_base 552..553: mod_base = OTHER; note = Pseudouridine
  modified_base 554: mod_base = OTHER; note = 5-methyl-cytidine
  modified_base 556..558: mod_base = OTHER; note = 5-methyl-cytidine
  modified_base 559: mod_base = OTHER; note = Pseudouridine
  modified_base 561..562: mod_base = OTHER; note = Pseudouridine
  modified_base 563: mod_base = OTHER; note = 5-methyl-cytidine
  source 1..567: mol_type = other RNA